(19) United States
(12) Patent Application Publication
(10) Pub. No.: US 2020/0360381 A1
(43) Pub. Date: Nov. 19, 2020

(54) COMPOUNDS FOR TREATING DENGUE VIRUS INFECTION AND OTHER VIRAL INFECTIONS

(71) Applicants: Dana-Farber Cancer Institute, Inc., Boston, MA (US); President and Fellows of Harvard College, Cambridge, MA (US)

(72) Inventors: Priscilla L. Yang, Boston, MA (US); Wenlong Lian, Lebanon, NH (US); Nathanael S. Gray, Boston, MA (US); Nicholas Paul Kwiatkowski, Brookline, MA (US); Jinhua Wang, Winchester, MA (US); Jaebong Jang, Seo-gu Daejeon (KR)

(73) Assignees: Dana-Farber Cancer Institute, Inc., Boston, MA (US); President and Fellows of Harvard College, Cambridge, MA (US)

(21) Appl. No.: 16/614,107

(22) PCT Filed: May 15, 2018
(86) PCT No.: PCT/US2018/032849

§ 371 Date: Nov. 15, 2019

Related U.S. Application Data
(60) Provisional application No. 62/506,588, filed on May 15, 2017.

(51) Int. Cl. A61K 31/5025; A61K 31/4045; A61K 31/416; A61K 31/47; A61K 31/277; A61K 31/167; A61K 31/53; A61P 31/16

(52) U.S. Cl. CPC A61K 31/5025; A61K 31/4045; A61K 31/416; A61P 31/16; A61K 31/277; A61K 31/167; A61K 31/53; A61K 31/47

(57) ABSTRACT

The present disclosure provides antiviral (e.g., anti-Dengue virus) agents, such as compounds of Formula (I). Also provided are pharmaceutical compositions and kits of the compounds of Formula (I). Further provided are methods and uses of the antiviral agents (e.g., the compounds of Formula (I); and compounds K786-9739, S4105, C200-5340, G199-0398, C200-9144, S7337, S1633, and C066-4182, and derivatives thereof) for treating or preventing a viral infection (e.g., Dengue fever). The antiviral agents may inhibit the entry of a virus into a cell by, e.g., inhibiting the fusion between the envelope of the virus and the membrane of the cell.

### RELATED APPLICATIONS

The present application claims priority under 35 U.S.C. § 119(e) to U.S. provisional application. U.S. Ser. No. 62/506,588, filed May 15, 2017, which is incorporated herein by reference.

### GOVERNMENT SUPPORT

This invention was made with government support under Grant Numbers R56AI095499, R01AI095499, and U19AI109740 awarded by the National Institutes of Health. The government has certain rights in the invention.

### BACKGROUND OF THE INVENTION

Dengue virus (DENV or DV) is a mosquito-borne virus from the genus Flavivirus. The genus Flavivirus also includes yellow fever virus, West Nile virus, Japanese encephalitis virus, and Zika virus. Over 300 million Dengue infections occur annually¹, resulting in disease that include Dengue hemorrhagic fever (DHF) and Dengue shock syndrome (DSS). Geographic spread of the Aedes mosquito species that transmit Dengue and Zika viruses and Zika's recent explosive emergence in the Western Hemisphere have heightened the need for countermeasures that can reduce transmission and prevent or lessen infections caused by these viruses. While the first Dengue vaccine, Dengvaxia, has been approved for use in several countries, its heterogeneous efficacy profile²⁻⁵ and evidence that it significantly increases risk of hospitalization for young children⁶ show a need for on-going studies, such as studies to determine how it can be used to protect at-risk subjects while minimizing exacerbation of disease due to antibody-dependent enhancement (ADE) of infection upon subsequent infection with Dengue⁷⁻⁹ or other cross-reacting flaviviruses, such as Zika virus¹⁰⁻¹⁴. There have been no approved small molecule antivirals against DENY or other flaviviruses that inhibit the DENV protease and polymerase enzymes^{15,16}. Therefore, there is a need for the development of novel anti-DENY agents and antiviral agents against other viruses.

### SUMMARY OF THE INVENTION

The present disclosure provides compounds of Formula (I):

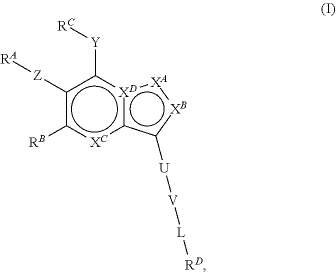

and pharmaceutically acceptable salts, solvates, hydrates, polymorphs, co-crystals, tautomers, stereoisomers, isotopically labeled derivatives, and prodrugs thereof, as antiviral agents, e.g., anti-Dengue virus agents, wherein X^{A}, X^{B}, X^{C}, X^{D}, Y, Z, U, V, L, R^{A}, R^{B}, R^{C}, and R^{D} are as described herein.

Exemplary compounds of Formula. (I) include the compounds of any one of the formulae:

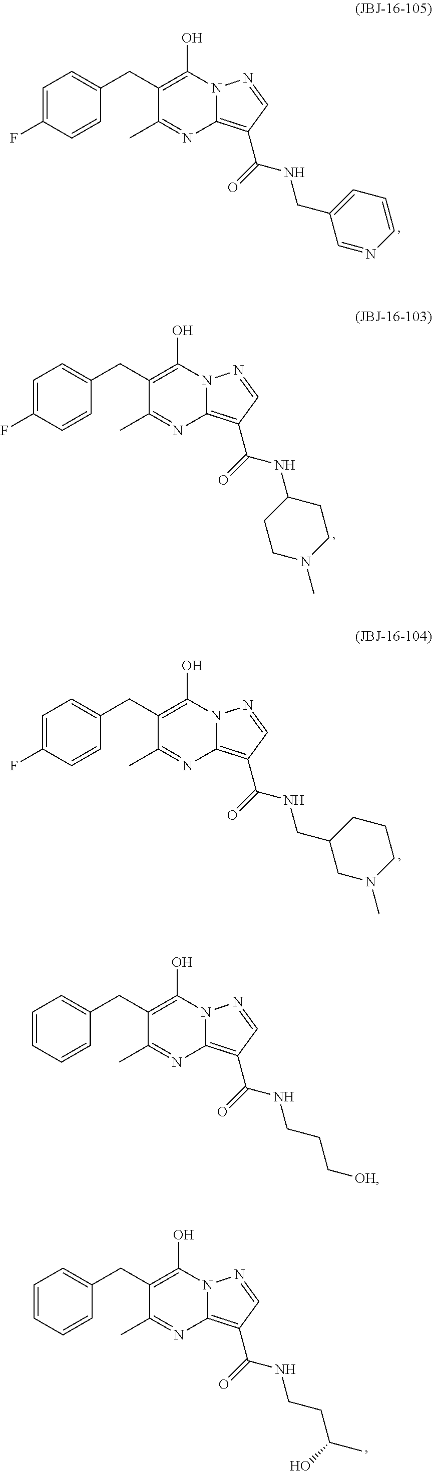 [SMILES: Cc1nc2c(C(=O)NCc3cccnc3)cnn2c(O)c1Cc1ccc(F)cc1 ; Cc1nc2c(C(=O)NC3CCN(C)CC3)cnn2c(O)c1Cc1ccc(F)cc1 ; Cc1nc2c(C(=O)NCC3CCCN(C)C3)cnn2c(O)c1Cc1ccc(F)cc1 ; Cc1nc2c(C(=O)NCCCO)cnn2c(O)c1Cc1ccccc1 ; Cc1nc2c(C(=O)NCC[C@H](C)O)cnn2c(O)c1Cc1ccccc1]

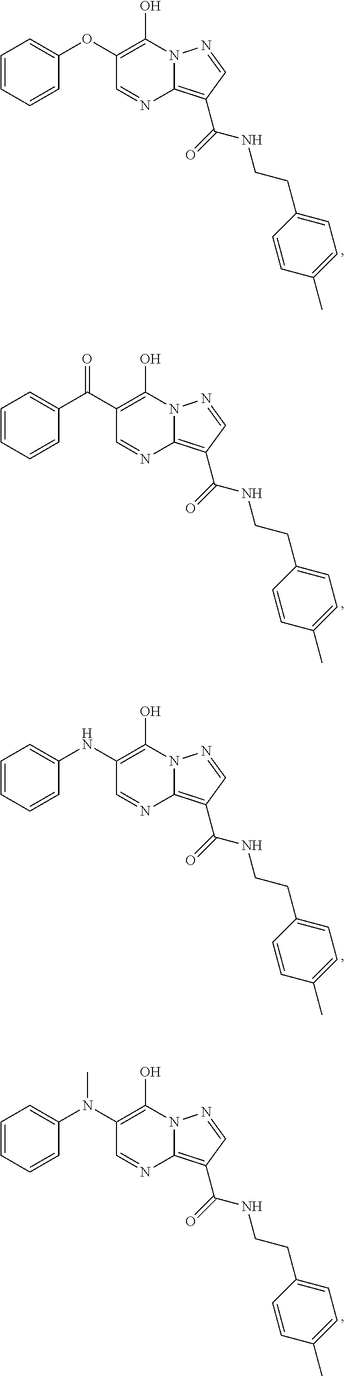 [SMILES: Cc1ccc(CCNC(=O)c2cnn3c(O)c(Oc4ccccc4)cnc23)cc1 ; Cc1ccc(CCNC(=O)c2cnn3c(O)c(C(=O)c4ccccc4)cnc23)cc1 ; Cc1ccc(CCNC(=O)c2cnn3c(O)c(Nc4ccccc4)cnc23)cc1 ; Cc1ccc(CCNC(=O)c2cnn3c(O)c(N(C)c4ccccc4)cnc23)cc1]

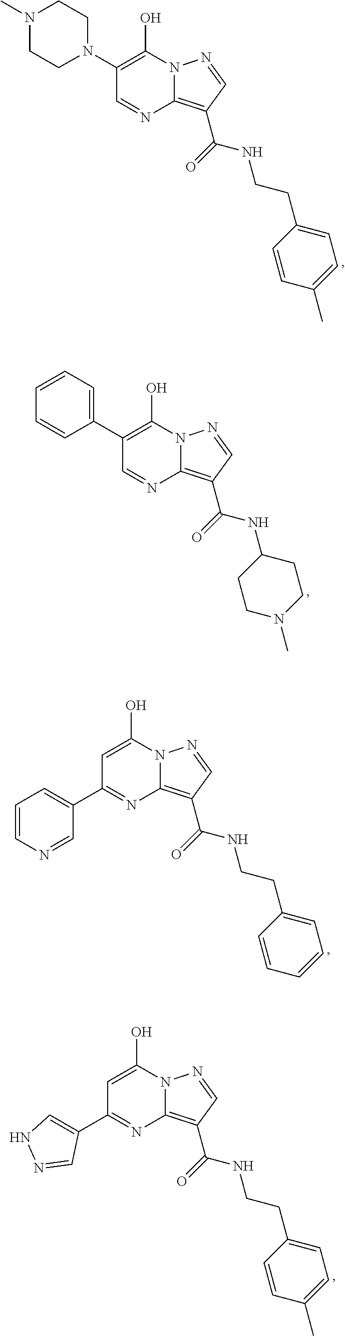 [SMILES: Cc1ccc(CCNC(=O)c2cnn3c(O)c(N4CCN(C)CC4)cnc23)cc1 ; CN1CCC(NC(=O)c2cnn3c(O)c(-c4ccccc4)cnc23)CC1 ; O=C(NCCc1ccccc1)c1cnn2c(O)cc(-c3cccnc3)nc12 ; Cc1ccc(CCNC(=O)c2cnn3c(O)cc(-c4cn[nH]c4)nc23)cc1]

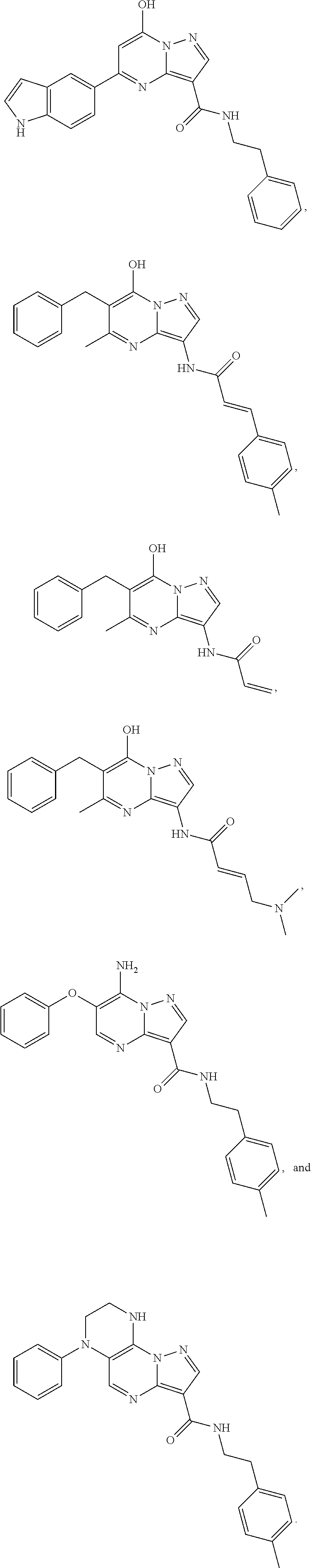 [SMILES: O=C(NCCc1ccccc1)c1cnn2c(O)cc(-c3ccc4[nH]ccc4c3)nc12 ; Cc1ccc(/C=C/C(=O)Nc2cnn3c(O)c(Cc4ccccc4)c(C)nc23)cc1 ; C=CC(=O)Nc1cnn2c(O)c(Cc3ccccc3)c(C)nc12 ; Cc1nc2c(NC(=O)/C=C/CN(C)C)cnn2c(O)c1Cc1ccccc1 ; Cc1ccc(CCNC(=O)c2cnn3c(N)c(Oc4ccccc4)cnc23)cc1 ; Cc1ccc(CCNC(=O)c2cnn3c4c(cnc23)N(c2ccccc2)CCN4)cc1]

Additional exemplary compounds of Formula (I) include the compounds of any one of the formulae:

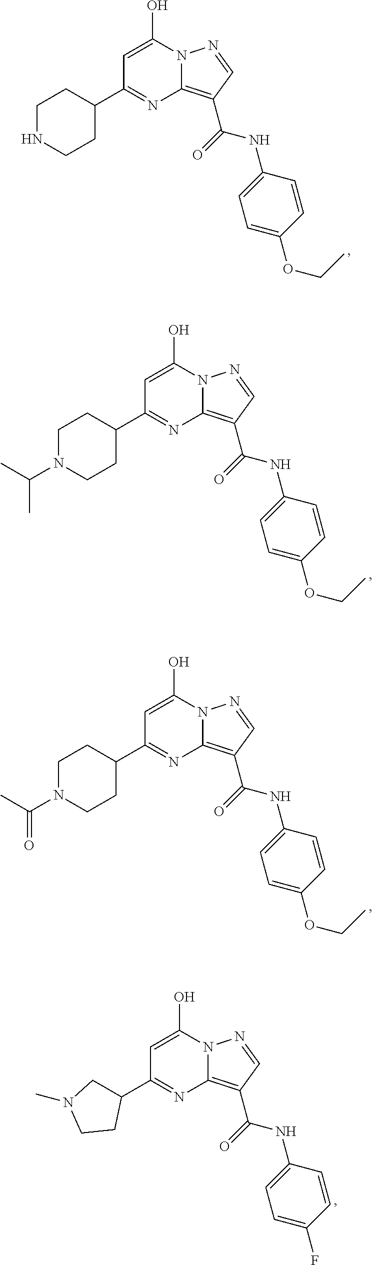

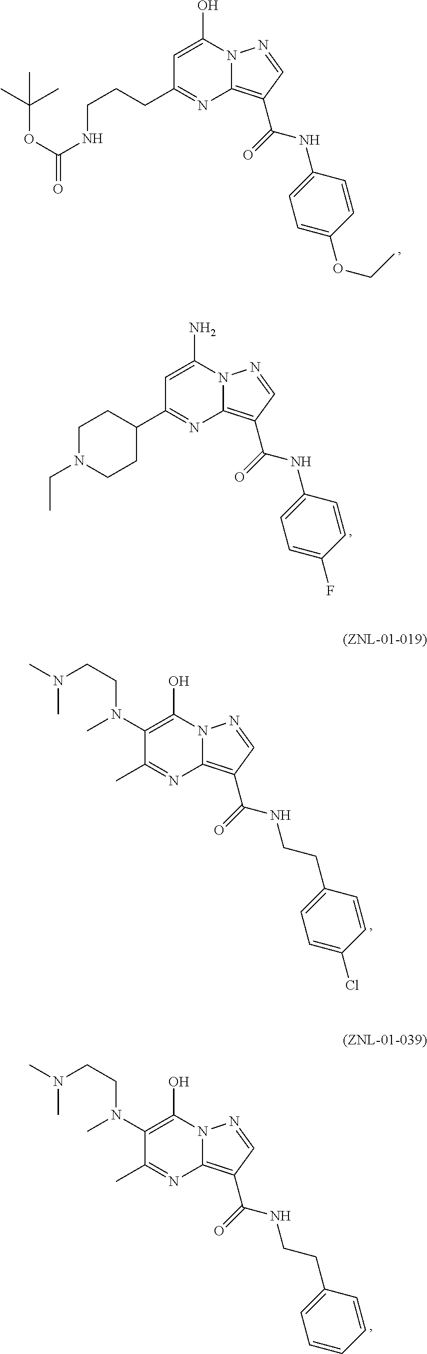

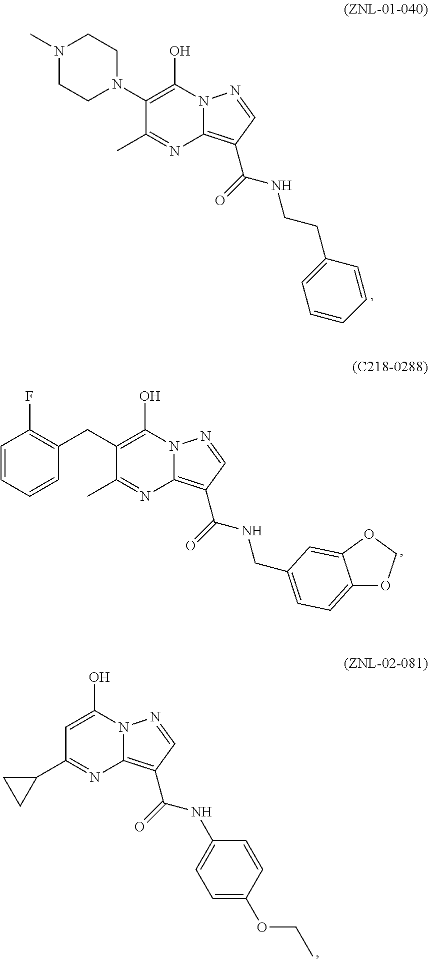 [SMILES: Cc1nc2c(C(=O)NCCc3ccccc3)cnn2c(O)c1N1CCN(C)CC1 ; Cc1nc2c(C(=O)NCc3ccc4c(c3)OCO4)cnn2c(O)c1Cc1ccccc1F ; CCOc1ccc(NC(=O)c2cnn3c(O)cc(C4CC4)nc23)cc1]

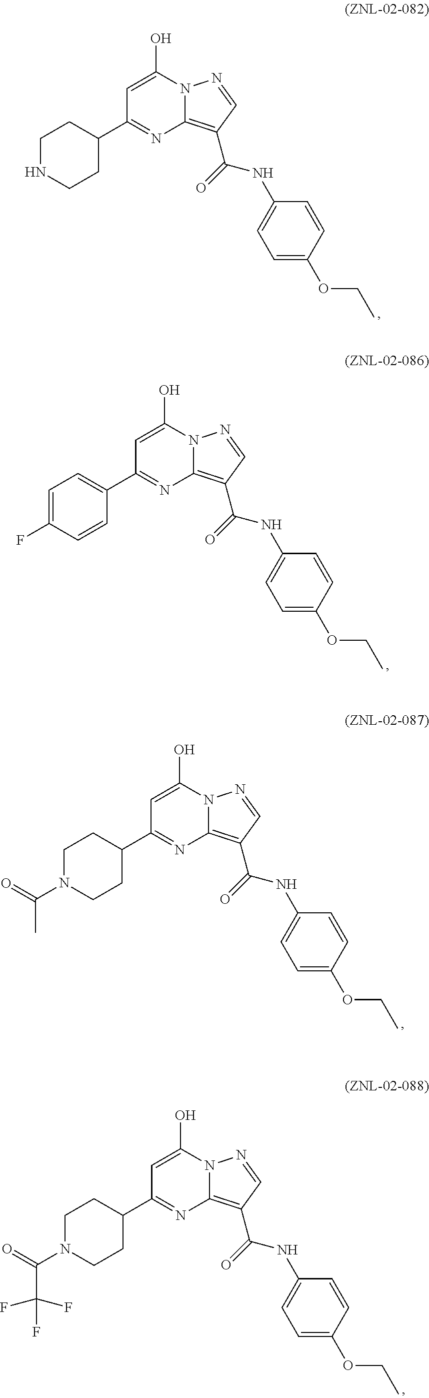 [SMILES: CCOc1ccc(NC(=O)c2cnn3c(O)cc(C4CCNCC4)nc23)cc1 ; CCOc1ccc(NC(=O)c2cnn3c(O)cc(-c4ccc(F)cc4)nc23)cc1 ; CCOc1ccc(NC(=O)c2cnn3c(O)cc(C4CCN(C(C)=O)CC4)nc23)cc1 ; CCOc1ccc(NC(=O)c2cnn3c(O)cc(C4CCN(C(=O)C(F)(F)F)CC4)nc23)cc1]

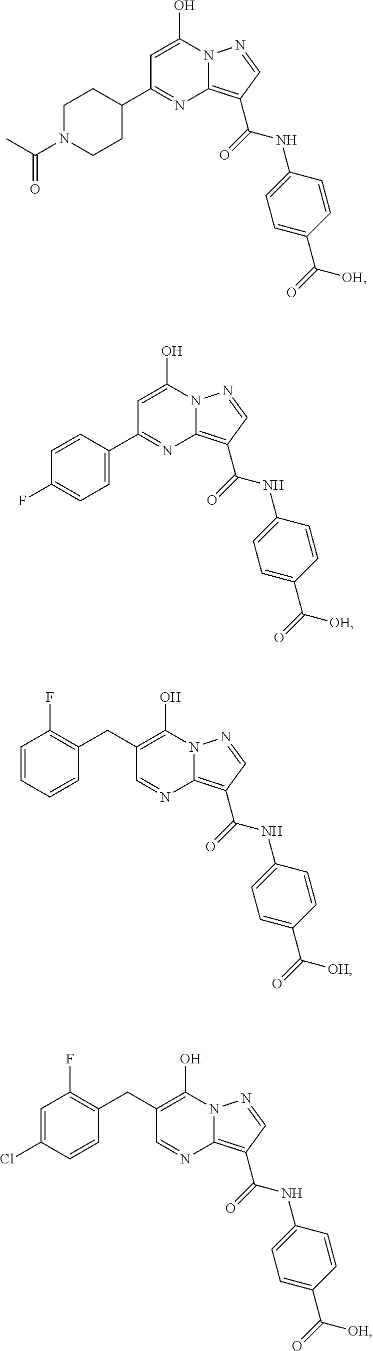 [SMILES: CC(=O)N1CCC(c2cc(O)n3ncc(C(=O)Nc4ccc(C(=O)O)cc4)c3n2)CC1 ; O=C(O)c1ccc(NC(=O)c2cnn3c(O)cc(-c4ccc(F)cc4)nc23)cc1 ; O=C(O)c1ccc(NC(=O)c2cnn3c(O)c(Cc4ccccc4F)cnc23)cc1 ; O=C(O)c1ccc(NC(=O)c2cnn3c(O)c(Cc4ccc(Cl)cc4F)cnc23)cc1]

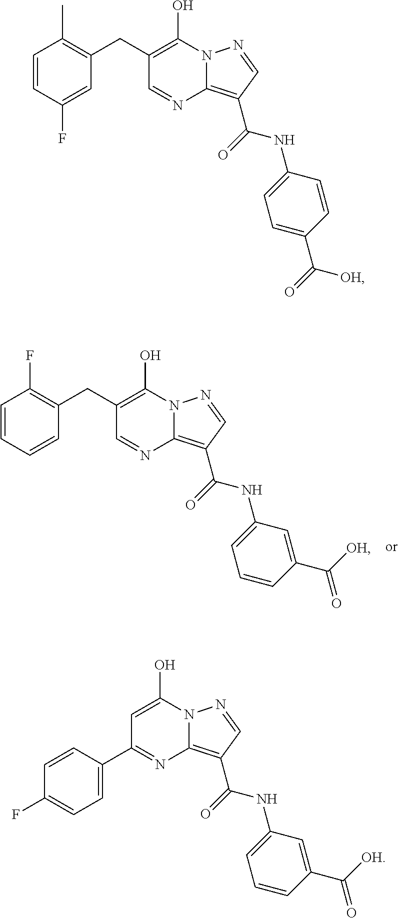 [SMILES: Cc1ccc(F)cc1Cc1cnc2c(C(=O)Nc3ccc(C(=O)O)cc3)cnn2c1O ; O=C(O)c1cccc(NC(=O)c2cnn3c(O)c(Cc4ccccc4F)cnc23)c1 ; "or" ; O=C(O)c1cccc(NC(=O)c2cnn3c(O)cc(-c4ccc(F)cc4)nc23)c1]

Also provided herein are pharmaceutical compositions and kits of the compounds of Formula (I).

Further provided herein are methods and uses of the compounds of Formula (I), and pharmaceutical compositions and kits thereof; and compounds of any one of the formulae:

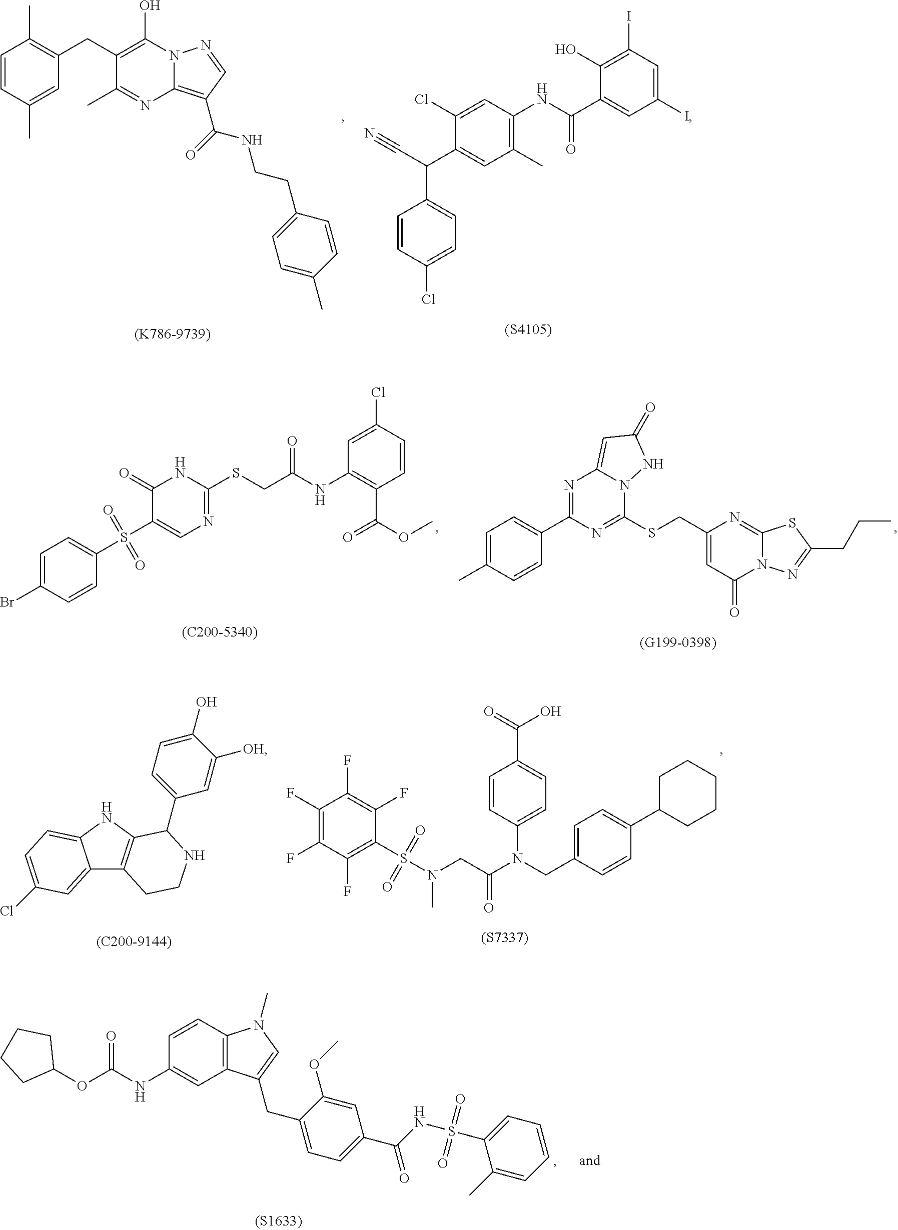

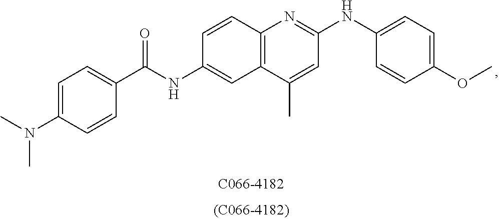

and pharmaceutically acceptable salts, solvates, hydrates, polymorphs, co-crystals, tautomers, stereoisomers, derivatives (e.g., isotopically labeled derivatives), and prodrugs thereof, for treating or preventing a viral infection (e.g., Dengue fever). Further provided herein are methods and uses of a compound of the formula:

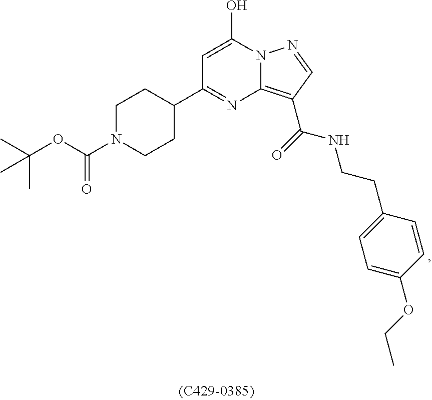 [SMILES: CCOc1ccc(CCNC(=O)c2cnn3c(O)cc(C4CCN(C(=O)OC(C)(C)C)CC4)nc23)cc1]

or compound C218-0288; or a pharmaceutically acceptable salt, solvate, hydrate, polymorph, co-crystal, tautomer, stereoisomer, derivative, or prodrug thereof, for treating or preventing a viral infection (e.g., Dengue fever).

The compounds described herein may inhibit the entry of a virus into a cell.

The compounds described herein may inhibit an envelope glycoprotein of the virus. The compounds described herein may inhibit the fusion between the envelope of the virus and the membrane of the cell.

Further provided herein are methods and uses of the compounds described herein for inhibiting the entry of a virus into a cell.

Further provided herein are methods and uses of the compounds described herein for inhibiting an envelope glycoprotein of a virus.

Compared to known antiviral agents, the compounds described herein may be more potent, wider spectrum (e.g., pan-serotype), more effective against viruses' resistance to known antiviral agents, less affected by viruses' mutations, and/or less toxic.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's advanced Organic Chemistry, 5^{th} Edition, John Wiley Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC), supercritical fluid chromatography (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGrawHill, NY, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

Unless otherwise provided, a formula depicted herein includes compounds that do not include isotopically enriched atoms and also compounds that include isotopically enriched atoms. Compounds that include isotopically enriched atoms may be useful as, for example, analytical tools, and/or probes in biological assays.

The term “aliphatic” includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups (e.g., halo, such as fluorine). As will be appreciated by one of ordinary skill in the art, “aliphatic” is intended herein to include alkyl, alkenyl, alkynyl, cycloalkyl, cycloalkenyl, and cycloalkynyl moieties.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight--chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”), In some embodiments, an alkyl group has 1 to 10 carbon atoms (“C₁₋₁₀ alkyl”), In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”), In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₁₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2 butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₂ alkyl (e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted ter t-butyl (tert-Bu or t-Bu), unsubstituted sec-butyl (sec-Bu or s-Bu), unsubstituted isobutyl (i-Bu)). In certain embodiments, the alkyl group is substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)). The attachment point of alkyl may be a single bond (e.g., as in —CH₃), double bond (e.g., as in ═CH₂), or triple bond (e.g., as in ≡CH). The moieties ═CH₂ and ≡CFI are also alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₁₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms, one or more (e.g., two three, or four, as valency permits) carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂) 1-propenyl (C₃) 2-propenyl (C₃) 1-butenyl (C₄), 2-butenyl (C₄) butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃,

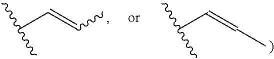

may be in the (E)- or (Z)-configuration.

“Alkynyl” refers to a radical of a straight-chain or branched hydrocarbon group having from 2 to 20 carbon atoms, one or more (e.g., two, three, or four, as valency permits) carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 13 ring carbon atoms (“C₃₋₁₃ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C3), cyclopropenyl (C₃), cyclobutyl (C₄), cyclobutenyl (C₄) cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”). Carbocyclyl can be saturated, and saturated carbocyclyl is referred to as “cycloalkyl.” In some embodiments, carbocyclyl is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. Carbocyclyl can be partially unsaturated. Carbocyclyl may include zero, one, or more (e.g., two, three, or four, as valency permits) C═C double bonds in all the rings of the carbocyclic ring system that are not aromatic or heteroaromatic. Carbocyclyl including one or more (e.g., two or three, as valency permits) C═C double bonds in the carbocyclic ring is referred to as “cycloalkenyl.” Carbocyclyl including one or more (e.g., two or three, as valency permits) C≡C triple bonds in the carbocyclic ring is referred to as “cycloalkynyl.” Carbocyclyl includes aryl. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl. In certain embodiments, the carbocyclyl is substituted or unsubstituted, 3- to 7-membered, and monocyclic. In certain embodiments, the carbocyclyl is substituted or unsubstituted, 5- to 13-membered, and bicyclic.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 13-membered non-aromatic ring system having ring carbon atoms and 1 to 4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system such as a bicyclic system (“bicyclic heterocyclyl”). A heterocyclyl group can be saturated or can be partially unsaturated. Heterocyclyl may include zero, one, or more (e.g., two, three, or four, as valency permits) double bonds in all the rings of the heterocyclic ring system that are not aromatic or heteroaromatic. Partially unsaturated heterocyclyl groups includes heteroaryl. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl. In certain embodiments, the heterocyclyl is substituted or unsubstituted, 3- to 7-membered, and monocyclic. In certain embodiments, the heterocyclyl is substituted or unsubstituted, 5- to 13-membered, and bicyclic.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include aziridinyl, oxiranyl, or thiiranyl. Exemplary 4-membered heterocyclyl groups containing one heteroatom include azetidinyl, oxetanyl and thietanyl. Exemplary 5-membered heterocyclyl groups containing one heteroatom include tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include dioxolanyl, oxasulfuranyl, disulfuranyl, and oxazolidin-2-one. Exemplary 5-membered heterocyclyl groups containing three heteroatoms include triazolinyl, oxadiazolinyl, and thiadiazolinyl. Exemplary 6-membered heterocyclyl groups containing one heteroatom include piperidinyl, tetrahydropyranyl, dihydropyridinyl, and thianyl. Exemplary 6-membered heterocyclyl groups containing two heteroatoms include piperazinyl, morpholinyl, dithianyl, and dioxanyl. Exemplary 6-membered heterocyclyl groups containing two heteroatoms include triazinanyl. Exemplary 7-membered heterocyclyl groups containing one heteroatom include azepanyl, oxepanyl and thiepanyl. Exemplary 8-membered heterocyclyl groups containing one heteroatom include azocanyl, oxecanyl, and thiocanyl. Exemplary 5-membered heterocyclyl groups fused to a C₆ aryl ring (also referred to herein as a 5,6-bicyclic heterocyclic ring) include indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 n electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include pyrrolyl, furanyl and thiophenyl. Exemplary 5-membered heteroaryl groups containing two heteroatoms include imidazolyl, pyrazolyl, oxazolyl, isoxazolyl, thiazolyl, and isothiazolyl. Exemplary 5-membered heteroaryl groups containing three heteroatoms include triazolyl, oxadiazolyl, and thiadiazolyl. Exemplary 5-membered heteroaryl groups containing four heteroatoms include tetrazolyl. Exemplary 6-membered heteroaryl groups containing one heteroatom include pyridinyl. Exemplary 6-membered heteroaryl groups containing two heteroatoms include pyridazinyl, pyrimidinyl, and pyrazinyl. Exemplary 6-membered heteroaryl groups containing three or four heteroatoms include triazinyl and tetrazinyl, respectively. Exemplary 7-membered heteroaryl groups containing one heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, aliphatic, alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include halogen, —CN, —NO₂, —N₃, —SO₂H, —SO₃H, —OH, —OR^{aa}, —N(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂. —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —P(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl. C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C_{1f}.alkyl), —OC(═NH)(C₁₋₄ alkyl), —OC(═NH)OC₁₋₆ alkyl. —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂. —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl), —OP(═O)(OC₁₋₆ alkyl)₂, C₁₋₆ alkyl, C₁₋₆ perhaloalkyl, C₂₋₆ alkenyl. C₂₋₆ alkynyl, heteroC₁₋₆alkyl, heteroC₂₋₆ alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, the carbon atom substituents are independently halogen, substituted (e.g., substituted with one or more halogen) or unsubstituted C₁₋₆alkyl, —OR. —SR^{aa}, —N(R^{bb})₂, —CN, —SCN, —NO₂, —C(═O)R^{aa}, —CO₂R, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₆alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₆alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group. In certain embodiments, the carbon atom substituents are independently halogen, substituted (e.g., substituted with one or more halogen) or unsubstituted C₁₋₆alkyl, —OR^{aa}, —SR^{aa}, —N(R^{bb})₂, —CN, —SCN, or —NO₂. In certain embodiments, the carbon atom substituents are independently halogen, substituted (e.g., substituted with one or more halogen moieties) or unsubstituted C₁ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₆alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁ alkyl, or a nitrogen protecting group.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₆ alkyl, or an oxygen protecting group when attached to an oxygen atom, and each R^{bb} is independently hydrogen, substituted (e.g., substituted with one or more halogen) or unsubstituted C₁₋₆ alkyl, or a nitrogen protecting group. In certain embodiments, the nitrogen atom substituents are independently substituted (e.g., substituted with one or more halogen) or unsubstituted C₁₋₆ alkyl or a nitrogen protecting group.

In certain embodiments, the substituent present on a nitrogen atom is a nitrogen protecting group (also referred to as an amino protecting group). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc). 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate. N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, I-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include phenothiazinyl-(10)-acyl derivative, N′-p-toluenesulfonylaminoacyl derivative. N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide. N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, a nitrogen protecting group is Bn, Boc, Cbz, Fmoc, trifluoroacetyl, triphenylmethyl, acetyl, or Ts.

In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group. In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the oxygen atom substituents are independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, <(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-4′-bromophenacyloxyphenyl)diphenylmethyl, 4,4′,4″-tris(4,5-dichlorophthalimidophenyl)methyl, 4,4′,4″-tris(levulinoyloxvphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, an oxygen protecting group is silyl. TBDPS, TBDMS, TIPS, TES, TMS, MOM, THP, t-Bu. Bn, allyl, acetyl, pivaloyl, or benzoyl.

In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group. In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the sulfur atom substituents are independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). Sulfur protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

The “molecular weight” of —R, wherein —R is any monovalent moiety, is calculated by subtracting the atomic weight of a hydrogen atom from the molecular weight of the molecule R—H. The “molecular weight” of -L-, wherein -L- is any divalent moiety, is calculated by subtracting the combined atomic weight of two hydrogen atoms from the molecular weight of the molecule H-L-H.

In certain embodiments, the molecular weight of a substituent is lower than 200, lower than 150, lower than 100, lower than 50, or lower than 25 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, and/or iodine atoms. In certain embodiments, a substituent consists of carbon, hydrogen, and/or fluorine atoms. In certain embodiments, a substituent does not comprise one or more, two or more, or three or more hydrogen bond donors. In certain embodiments, a substituent does not comprise one or more, two or more, or three or more hydrogen bond acceptors.

These and other exemplary substituents are described in more detail in the Detailed Description, Examples, and claims. The present disclosure is not intended to be limited in any manner by the above exemplary listing of substituents.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences (1977) 66:1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including. e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl. C₂-C₈ alkenyl, C₂-C₈ alkynyl, aryl, C₇-C₁₂ substituted aryl, and C₇-C₁₂ arylalkyl esters of the compounds described herein may be preferred.

The terms “composition” and “formulation” are used interchangeably.

A “subject” to which administration is contemplated includes humans (e.g., a male or female of any age group, e.g., a pediatric subject (e.g. infant, child, adolescent) or adult subject (e.g., young adult, middle-aged adult or senior adult)) and/or other non-human animals, for example mammals (e.g., primates (e.g. cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), reptiles, amphibians, and fish. In certain embodiments, the non-human animal is a mammal. The non-human animal may be a male or female at any stage of development. A non-human animal may be a transgenic animal.

“Condition.” “disease,” and “disorder” are used interchangeably herein. The conditions described herein include viral infections.

The term “viral infection” refers to an infectious disease caused at least in part by a virus.

The term “administer,” “administering,” or “administration” refers to implanting, absorbing, ingesting, injecting, inhaling, or otherwise introducing a compound described herein, or a composition thereof, in or on a subject.

“Treat,” “treating” and “treatment” encompasses an action that occurs while a subject is suffering from a condition which reduces the severity of the condition or retards or slows the progression of the condition (“therapeutic treatment”). “Treat,” “treating” and “treatment” also encompasses an action that occurs before a subject begins to suffer from the condition and which inhibits or reduces the severity of the condition (“prophylactic treatment”).

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects.

An “effective amount” of a compound refers to an amount sufficient to elicit the desired biological response, e.g., treat the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a compound is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “IC₉₀” refers to the concentration of an antiviral agent that reduces single-cycle viral yield by 10-fold.

The term “CC₅₀” refers to the concentration of an antiviral agent that causes 50% loss of cell viability.

The term “SI_{50/90}” refers to selectivity index, whose value is equal to the value of CC₅₀/IC₉₀.

### BRIEF DESCRIPTION OF THE DRAWINGS

FIGS. 1A to 1E show the high throughput screening (HTS) for identifying and validating inhibitors of DENV (inhibitors) that target the envelop protein. FIG. 1A shows the primary and secondary screening flow-chart for identifying inhibitors of DENV envelop protein that bind in the βOG pocket. R1 and R2 refer to rounds 1 and 2, respectively, of the HTS. FIG. 1B shows exemplary results of an Amplified Luminescent Proximity Homogeneous Assay screen (AlphaScreen) assay for identifying inhibitors with concentration-dependent inhibitory activity. FIG. 1C shows exemplary results of an initial antiviral activity assay using select compounds at concentrations of 3 μM and 10 μM. FIG. 1D shows an exemplary mechanism of the AlphaScreen assay. FIG. 1E shows another exemplary mechanism of the AlphaScreen assay.

FIGS. 2A and 2B show the conformation of exemplary HTS hits. FIG. 2A shows the determination of the IC₉₀ value of compound K786-9739 with a DV2 infectivity assay. “PFU” refers to plaque-forming units. FIG. 2B shows that the IC₅₀ values of select compounds obtained from the AlphaScreen assay are well-correlated with the IC₉₀ values of the select compounds obtained from the DV2 infectivity assay.

FIG. 3 shows that structures of the compounds GNF2 and biotinylated GNF2 (GNF2-biotin).

FIG. 4 shows that the low pH-triggered transformation of E from pre-fusion dimer to post-fusion trimer catalyzes fusion of the viral and endosomal membranes. The major envelope glycoprotein (E) of the Dengue virus mediates viral attachment and entry by membrane fusion. The envelope glycoprotein (E) contains a hydrophobic pocket lined by residues that influence the pH threshold for fusion. The pocket, which can bind hydrophobic ligands, opens and closes through a conformational shift in a β-hairpin at the interface between two domains. Small-molecule inhibitors of dengue (and other flaviviruses) can play into this structural pathway for fusion-activating transition. See, e.g., Proc. Natl. Acad. Sci., 2003, 100 (12), 6986-6991).

FIGS. 5A and 5B show exemplary specificity of select compounds. FIG. 5A shows exemplary non-specific inhibition of VSV by the select compounds. FIG. 5B shows exemplary non-specific interaction with unrelated protein. J. Med. Chem., 2015, 58 (17), 7076-7087.

FIGS. 6A to 6C show exemplary activity of select compounds. FIG. 6A: exemplary data for select compounds. FIG. 6B shows that the activity of the selected compounds in the Alphascreen was well-correlated with inhibition of DENV infectivity. FIG. 6C shows that the binding affinity of the select compounds to the E protein was also well-correlated with inhibition of DENV infectivity.

### DETAILED DESCRIPTION OF CERTAIN EMBODIMENTS OF THE INVENTION

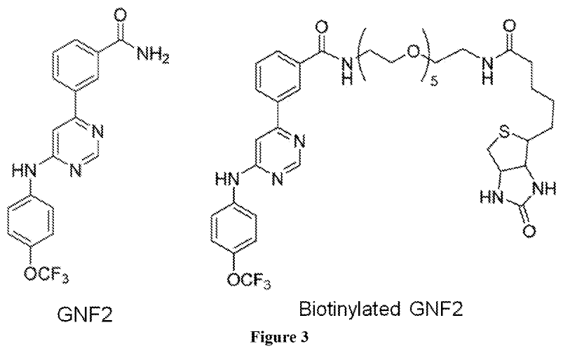

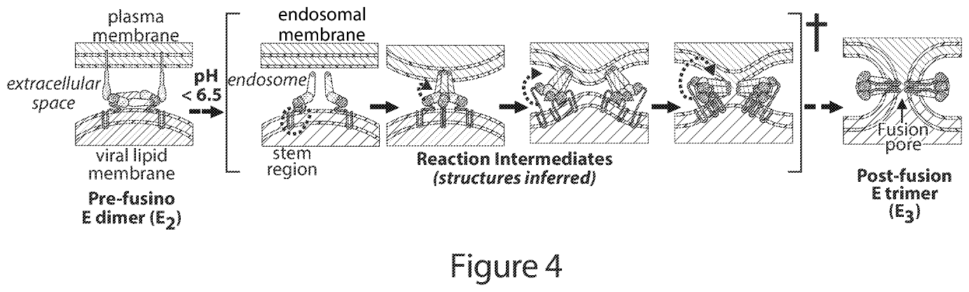

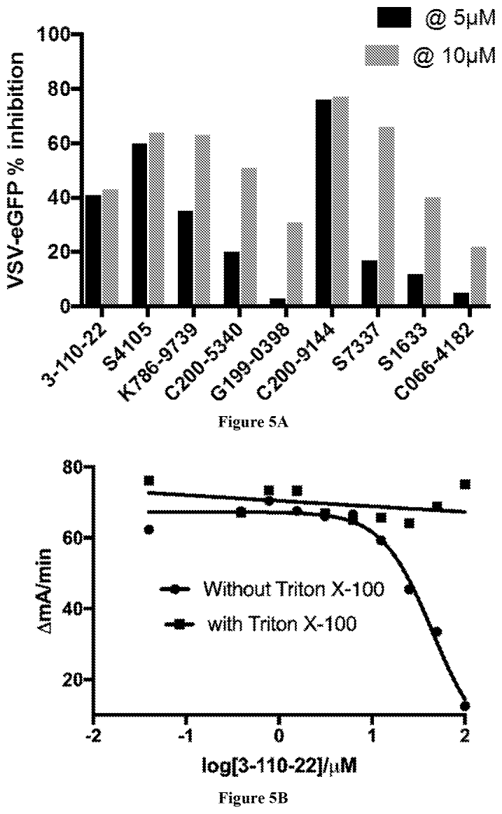

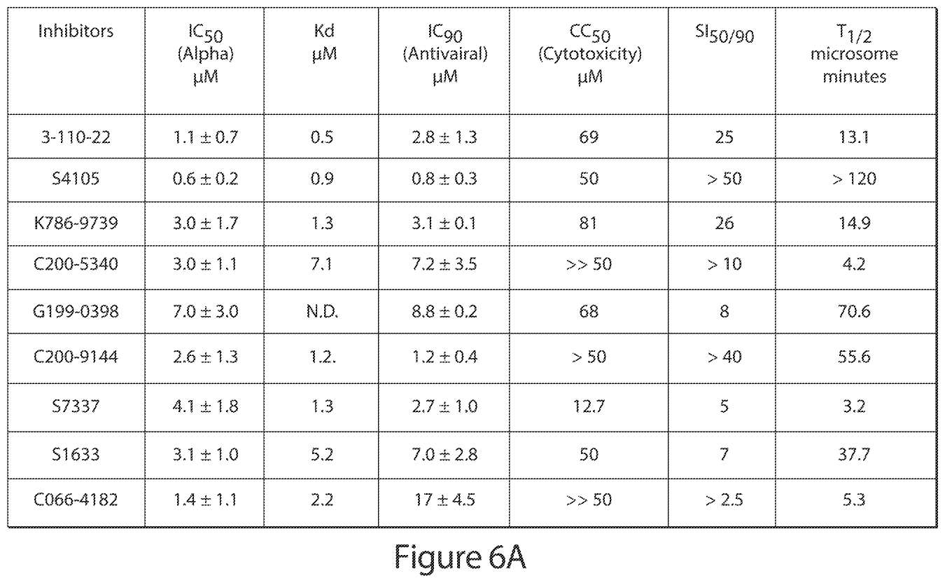

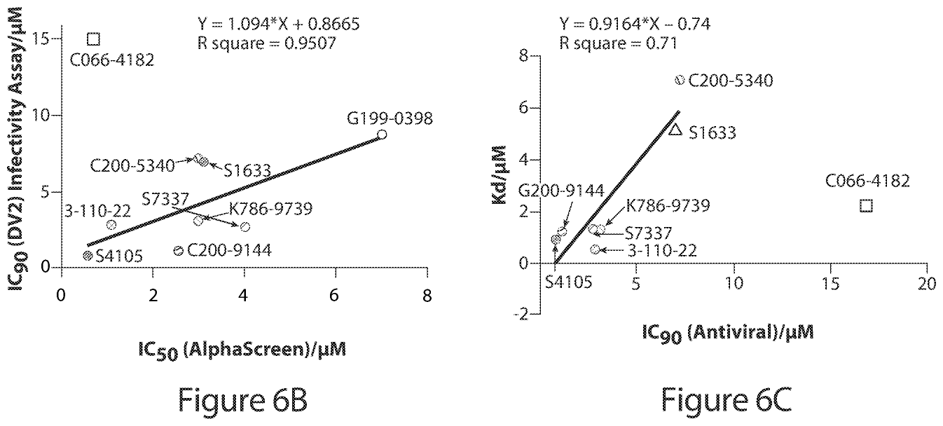

The envelope glycoprotein (E) of a virus (e.g., DENV) on the virion surface presents a target for direct-acting antiviral agents that act at the earliest stage of the viral life cycle and thus mimic the humoral immune system. Viral envelope glycoproteins catalyze fusion of viral and cellular membranes, an obligate step in entry of enveloped viruses. Neutralizing antibodies that block fusion by binding to viral envelope proteins demonstrate that this may be an effective antiviral strategy. However, there are few examples of antivirals that have this mode of action. We established a high-throughput competitive AlphaScreen (amplified luminescent proximity homogeneous assay) utilizing a biotinylated derivative of GNF2 (FIG. 3) to identify additional compound classes that may inhibit viral entry into a cell by targeting the envelope glycoprotein of the virus. Via high-throughput screening with this assay, we identified compounds that may inhibit DENV in cell culture, with excellent correlation of activity in the AlphaScreen with antiviral potency. Since prior efforts to target DENV envelope glycoprotein have relied on in silico and phenotypic screens, the assays described herein may provide tools to discover inhibitors of envelope glycoproteins, to define the structure-activity relationship (SAR) for antiviral activity mediated by this target, and to develop inhibitors (e.g., small molecule inhibitors) of viral entry as potential antiviral (e.g., anti-DNEV) agents.

Small molecules that target the viral glycoprotein may be of interest because they have the potential to engage their target extracellularly and to block the viral replication cycle at its earliest step. Validation of this antiviral strategy is provided by the humoral immune response to many viruses. The surface of the mature Dengue virion is covered by 90 prefusion dimers of the viral envelope glycoprotein. A soluble ectodomain comprising the envelope glycoprotein's three globular domains (I, II, and III) connects to a transmembrane anchor through a membrane-proximal “stem” region. The conserved fusion loop located at the tip of domain II of each monomer is buried in the interface between domains I and III of the partner monomer¹⁷⁻¹⁹. Viral entry is initiated by engagement of the envelope glycoprotein with attachment factors on the plasma membrane of the host cell, followed by uptake of the virion by a clathrin-dependent process²⁰⁻²². Acidification of the endosomal compartment is the physiological trigger for significant structural changes leading to reorganization and refolding of the envelope glycoprotein as a postfusion trimer²³⁻²⁵. This structural transformation induces fusion of the viral and endosomal membranes and creates a pore that allows escape of the viral nucleocapsid into the host cytosol where the viral RNA genome can be expressed.

Small molecules that inhibit Dengue virus entry by binding the envelope glycoprotein and/or by preventing fusion have been reported²⁶⁻³¹. However, the structural basis for their inhibitory activities has not been determined. Virtual and/or cellular based screening has been used by several groups to investigate the entry inhibitors of flaviviruses. No direct target-based HTS has been reported to identify specific Dengue fusion inhibitors.

### Compounds

In one aspect, the present disclosure provides compounds of Formula (I):

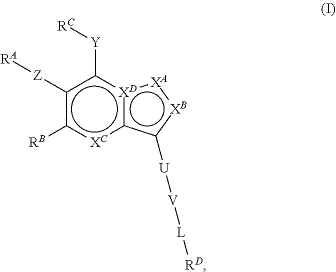

and pharmaceutically acceptable salts, solvates, hydrates, polymorphs, co-crystals, tautomers, stereoisomers, isotopically labeled derivatives, and prodrugs thereof, as antiviral agents, e.g., anti-Dengue virus agents, wherein X^{A}, X^{B}, X^{C}, X^{D}, Y, Z, U, V, L, R^{A}, R^{B}, R^{C}, and R^{D} are as described herein.

Without wishing to be bound by theory, the compounds described herein may inhibit the entry of a virus into a cell. The compounds described herein may inhibit an envelope

glycoprotein of the virus. The compounds described herein may inhibit the fusion between the
envelope of the virus and the membrane of the cell. Further provided herein are methods and uses of the compounds described herein for inhibiting the entry of a virus into a cell. Further provided herein are methods and uses of the compounds described herein for inhibiting an envelope glycoprotein of a virus.

In certain embodiments:

Z is a bond, O, S, —NR^{E}, or C(R^{F})₂;

R^{E} is hydrogen, substituted or unsubstituted, C₁₋₆ alkyl, or a nitrogen protecting group;

each instance of R^{F} is independently hydrogen, halogen, or substituted or unsubstituted, C₁₋₆ alkyl, or two instances of R^{F} are joined to form ═O;

when Z is a bond or C(R^{F})₂, R^{A} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;

each instance of R^{a} is independently hydrogen, substituted or unsubstituted, C₁₋₁₂ acyl, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, 3- to 7-membered, monocyclic heterocyclic ring, or substituted or unsubstituted, 5- to 6-membered, monocyclic heteroaryl ring;

when Z is O, S, or NR^{E}, R^{A} is hydrogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, a nitrogen protecting group when attached to a nitrogen atom, an oxygen protecting group when attached to an oxygen atom, or a sulfur protecting group when attached to a sulfur atom:

R^{B} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;

or R^{A} and R^{B} are joined to form substituted or unsubstituted, 3- to 7-membered, monocyclic carbocyclic ring, substituted or unsubstituted, 3- to 7-membered, monocyclic heterocyclic ring, substituted or unsubstituted, phenyl ring, or substituted or unsubstituted, 5-or 6-membered, monocyclic heteroaryl ring;

Y is C(R^{N})₂, O, S, or NR^{G}.

each instance of R^{N} is independently hydrogen, halogen, or substituted or unsubstituted, C₁₋₆ alkyl;

R^{G} is hydrogen, substituted or unsubstituted, C₁₋₆ alkyl, or a nitrogen protecting group;

R^{C} is hydrogen, substituted or unsubstituted, C₁₋₆ alkyl, a nitrogen protecting group when attached to a nitrogen group, an oxygen protecting group when attached to an oxygen atom, or a sulfur protecting group when attached to a sulfur atom;

or R^{C} and R^{E} are joined to form a substituted or unsubstituted, 3- to 7-membered, monocyclic heterocyclic ring;

or R^{C} and one instance of R^{F} are joined to form a substituted or unsubstituted, 3- to 7-membered, monocyclic heterocyclic ring;

X^{A} is N or NR^{H};

R^{H} is hydrogen, substituted or unsubstituted, C₁₋₆ alkyl, or a nitrogen protecting group;

X^{B} is N, NR^{M}, or CR^{J};

R^{M} is hydrogen, substituted or unsubstituted, C₁₋₆ alkyl, or a nitrogen protecting group,

R^{J} is hydrogen, halogen, or substituted or unsubstituted, C₁₋₆ alkyl;

X^{C} is N or CR^{L};

R^{L} is hydrogen, halogen, or substituted or unsubstituted, C₁₋₆ alkyl;

X^{D} is N or C:

provided that

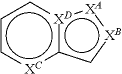

is bicyclic heteroaryl;

—U—V— is —C(═O)—NR^{K}— or —NR^{K}—C(═O)—;

R^{K} is hydrogen, substituted or unsubstituted, C₁₋₆ alkyl, or a nitrogen protecting group;

L is a bond, substituted or unsubstituted, C₁₋₆ alkylene, substituted or unsubstituted, C₂₋₆ alkenylene, or substituted or unsubstituted, C₂₋₆ alkynylene; and

R^{D} is hydrogen, substituted or unsubstituted, C₁₋₁₂ alkyl, substituted or unsubstituted. C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂. —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;

or R^{D} and R^{K} are joined to form a substituted or unsubstituted, 3- to 7-membered, monocyclic heterocyclic ring, or substituted or unsubstituted, 5- to 6-membered, monocyclic heteroaryl ring.

In certain embodiments:

Z is a bond, O, S, —NR^{E}, or C(R^{F})₂;

R^{E} is hydrogen, substituted or unsubstituted, C₁₋₆ alkyl, or a nitrogen protecting group;

each instance of R^{F} is independently hydrogen, halogen, or substituted or unsubstituted, C₁₋₆ alkyl, or two instances of R^{F} are joined to form ═O

when Z is a bond or C(R^{F})₂, R^{A} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;

each instance of R^{a} is independently hydrogen, substituted or unsubstituted, C₁₋₁₂ acyl, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, 3- to 7-membered, monocyclic heterocyclic ring, or substituted or unsubstituted, 5- to 6-membered, monocyclic heteroaryl ring:

when Z is O, S, or NR^{E}, R^{A} is hydrogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, a nitrogen protecting group when attached to a nitrogen atom, an oxygen protecting group when attached to an oxygen atom, or a sulfur protecting group when attached to a sulfur atom:

R^{B} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;

provided that at least one of R^{A} and R^{B} is substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl;

Y is O, S, or NR^{G};

R^{G} is hydrogen, substituted or unsubstituted, C₁₋₆ alkyl, or a nitrogen protecting group;

R^{C} is hydrogen, substituted or unsubstituted, C₁₋₆ alkyl, a nitrogen protecting group when attached to a nitrogen group, an oxygen protecting group when attached to an oxygen atom, or a sulfur protecting group when attached to a sulfur atom;

or R^{C} and R^{E} are joined to form a substituted or unsubstituted, 3- to 7-membered, monocyclic heterocyclic ring:

or R^{C} and one instance of R^{F} are joined to form a substituted or unsubstituted, 3- to 7-membered, monocyclic heterocyclic ring;

X^{A} is N or NR^{H}.

R^{H} is hydrogen, substituted or unsubstituted, C₁₋₆ alkyl, or a nitrogen protecting group;

X^{B} is N, NR^{M}, or CR^{J};

R^{M} is hydrogen, substituted or unsubstituted, C₁₋₆ alkyl, or a nitrogen protecting group;

R^{J} is hydrogen, halogen, or substituted or unsubstituted, C₁₋₆ alkyl:

X^{C} is N or CR^{L};

R^{L} is hydrogen, halogen, or substituted or unsubstituted, C₁₋₆ alkyl;

X^{D} is N or C;

provided that

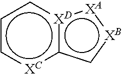

is bicyclic heteroaryl;

—U—V— is —C(═O)—NR^{K}— or —NR^{K}—C(═O)—;

R^{K} is hydrogen, substituted or unsubstituted, C₁₋₆ alkyl, or a nitrogen protecting group;

L is a bond, substituted or unsubstituted, C₁₋₆ alkylene, substituted or unsubstituted, C₂₋₆ alkenylene, or substituted or unsubstituted, C₂₋₆ alkynylene; and

R^{D} is hydrogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl;

provided that the compound is not of the formula:

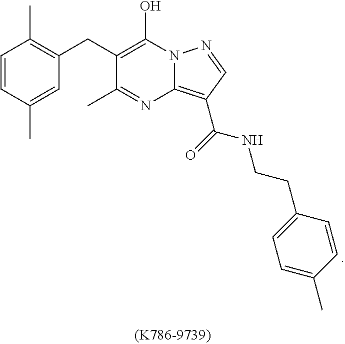 [SMILES: Cc1ccc(CCNC(=O)c2cnn3c(O)c(Cc4cc(C)ccc4C)c(C)nc23)cc1]

In certain embodiments, the compound of Formula (I) is not K786-9739. In certain embodiments, the compound of Formula (I) is not C429-0385. In certain embodiments, the compound of Formula (I) is not C218-0288. In certain embodiments, the compound of Formula (I) is not of the formula:

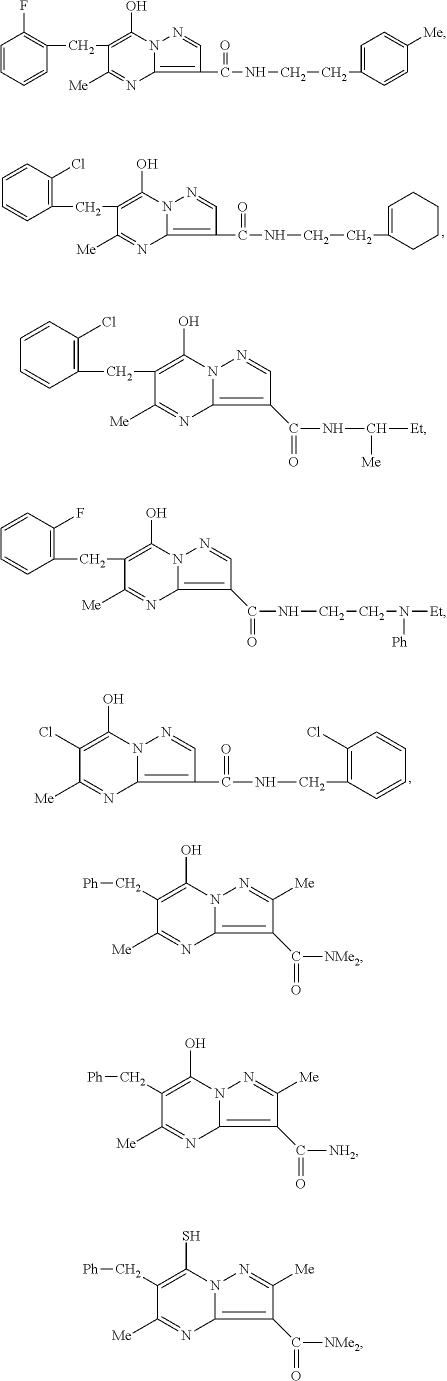

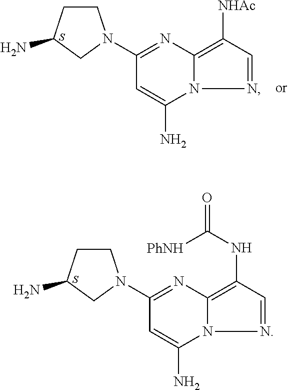

In certain embodiments, a compound of Formula (I) is of the formula:

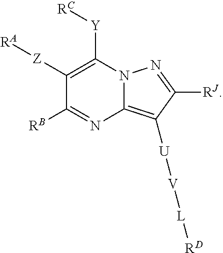

In certain embodiments, a compound of Formula (I) is of the formula:

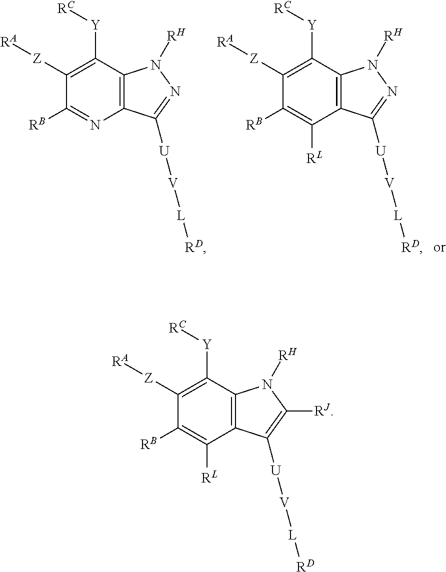

In certain embodiments, a compound of Formula (I) is of the formula:

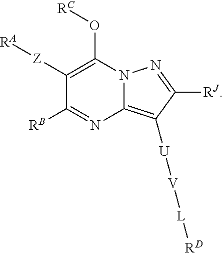

In certain embodiments a compound of Formula (I) is of the formula:

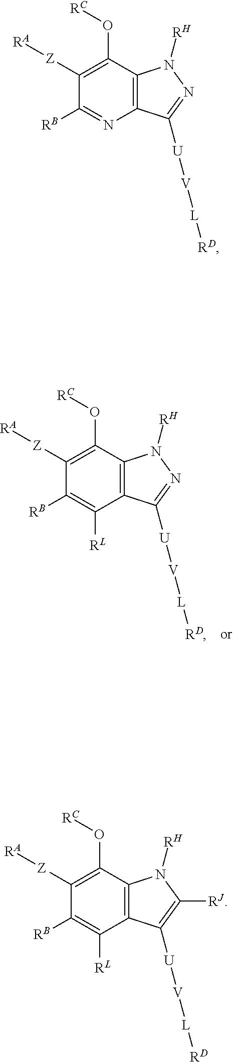

In certain embodiments, a compound of Formula (I) is of the formula:

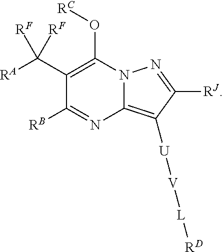

In certain embodiments, a compound of Formula (I) is of the formula:

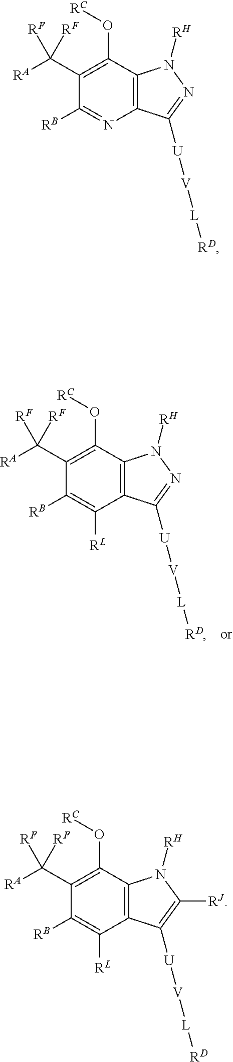

In certain embodiments, a compound of Formula (I) is of the formula:

wherein the compound is of the formula:
wherein:

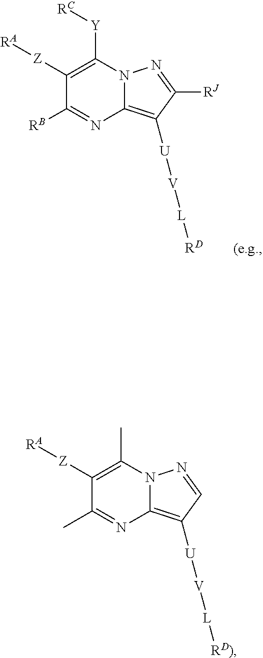

R^{B} is hydrogen, halogen, substituted or unsubstituted, C₁₋₆ alkyl, —OR^{a}, —N(R^{a})₂, —SR^{a}, or —CN; and

Y is C(R^{N})₂.

In certain embodiments, a compound of Formula (I) is of the formula:

wherein the compound is of the formula:

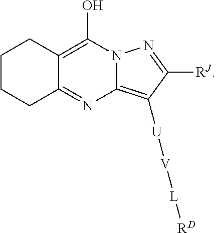

In certain embodiments, a compound of Formula (I) is of the formula:

wherein the compound is of the formula:

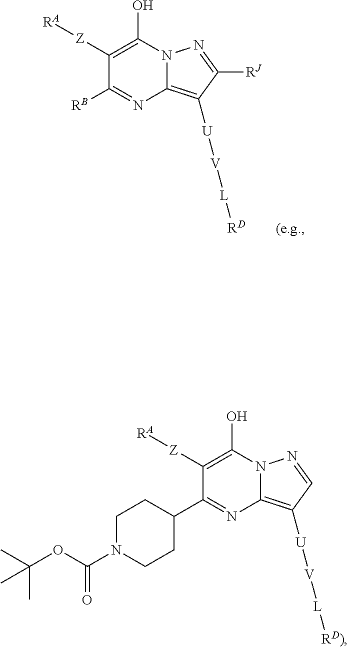

wherein R^{B} is substituted or unsubstituted 4-piperidinyl.

In certain embodiments, a compound of Formula (I) is of the formula:

wherein the compound is of the formula:

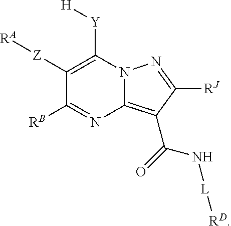

In certain embodiments, a compound of Formula (I) is of the formula:

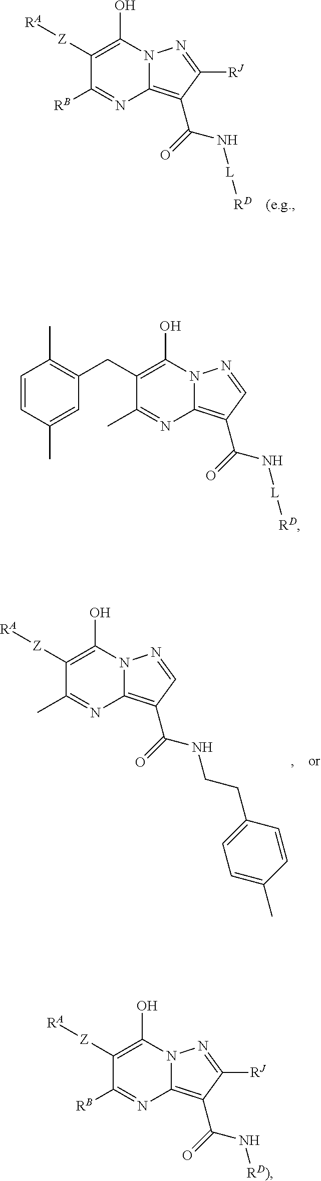

wherein:

R^{B} is hydrogen, halogen, substituted or unsubstituted, C₁₋₆ alkyl, —OR^{a}, —N(R^{a})₂, —SR^{a}, or —CN; and

R^{D} is substituted or unsubstituted phenyl.

In certain embodiments, a compound of Formula (I) is of the formula:

wherein the compound is of the formula:

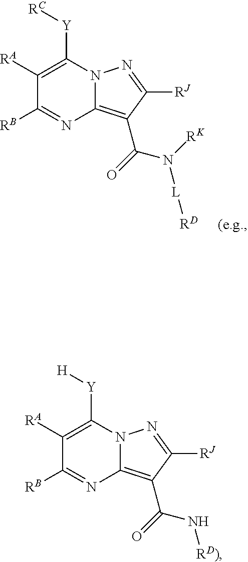

wherein:

R^{A} is hydrogen, halogen, substituted or unsubstituted, C₁₋₆, alkyl, —OR^{a}, —N(R)₂, —SR^{a}, or —CN;

R^{B} is substituted or unsubstituted C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂; and

Y is O or NR^{G}.

When Formula (I) includes two or more instances of a moiety, any two instances of the moiety may be the same or different from each other.

In certain embodiments, Z is a bond. In certain embodiments, Z is O, S, or NR^{E}. In certain embodiments, Z is O. In certain embodiments, Z is NR^{E} (e.g., NH or NMe). In certain embodiments, Z is C(R^{F}). In certain embodiments, Z is CH₂. In certain embodiments, Z is C(═O).

In certain embodiments, R^{E} is hydrogen, or substituted or unsubstituted C₁₋₆ alkyl. In certain embodiments, R^{E} is hydrogen. In certain embodiments, R^{E} is unsubstituted C₁₋₆ alkyl (e.g., Me). In certain embodiments, R^{E} is a nitrogen protecting group.

In certain embodiments, each instance of R^{F} is hydrogen. In certain embodiments, at least one instance of R^{F} is unsubstituted C₁₋₆ alkyl (e.g., Me). In certain embodiments, two instances of R^{F} are joined to form ═O.

In certain embodiments, R^{A} is hydrogen. In certain embodiments, when Z is a bond or C(R^{F})₂, R^{A} is halogen or substituted or unsubstituted, C₁₋₆ alkyl. In certain embodiments, when Z is a bond or C(R^{F})₂, R^{A} is halogen (e.g., F, Cl, or Br). In certain embodiments, R^{A} is substituted or unsubstituted, C₁₋₁₂ alkyl (e.g., substituted or unsubstituted, C₁₋₆ alkyl). In certain embodiments, R^{A} is C₁₋₆ alkyl substituted with one or more halogen (e.g., F). In certain embodiments, R^{A} is C₁₋₆ alkyl substituted with one or more —OR^{a} or —N(R^{a})₂. In certain embodiments, R^{A} is unsubstituted C₁₋₆ alkyl (e.g., Me). In certain embodiments. R^{A} is substituted or unsubstituted, C₂₋₁₂ alkenyl (e.g., substituted or unsubstituted, C₂₋₆ alkenyl) or substituted or unsubstituted, C₂₋₁₂ alkynyl (e.g., substituted or unsubstituted, C₂₋₆ alkynyl). In certain embodiments, R^{A} is substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl.

In certain embodiments, R^{A} is substituted or unsubstituted, 3- to 13-membered, monocyclic or bicyclic carbocyclyl. In certain embodiments, R^{A} is substituted or unsubstituted, 3- to 7-membered, monocyclic carbocyclyl. In certain embodiments. R^{A} is substituted or unsubstituted cyclopropyl. In certain embodiments, R^{A} is substituted or unsubstituted cyclobutyl, substituted or unsubstituted cyclopentyl, or substituted or unsubstituted cyclohexyl. In certain embodiments, R^{A} is substituted or unsubstituted, 5- to 13-membered, bicyclic carbocyclyl. In certain embodiments, R^{A} is substituted or unsubstituted, 5- to 13-membered, bicyclic carbocyclyl that is fused, spiro, or bridged.

In certain embodiments, R^{A} is substituted or unsubstituted, 3- to 13-membered, monocyclic or bicyclic heterocyclyl. In certain embodiments, R^{A} is substituted or unsubstituted, 3- to 7-membered (e.g., 6-membered), monocyclic heterocyclyl. In certain embodiments. R^{A} is substituted or unsubstituted oxetanyl, substituted or unsubstituted azetidinyl, substituted or unsubstituted tetrahydrofuranyl, substituted or unsubstituted pyrrolidinyl, substituted or unsubstituted tetrahydropyranyl, substituted or unsubstituted piperidinyl, substituted or unsubstituted morpholinyl, or substituted or unsubstituted piperazinyl. In certain embodiments, R^{A} is substituted or unsubstituted piperazinyl (e.g., substituted or unsubstituted 1-piperazinyl,

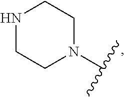 [SMILES: *N1CCNCC1]

or
(substituted or unsubstituted

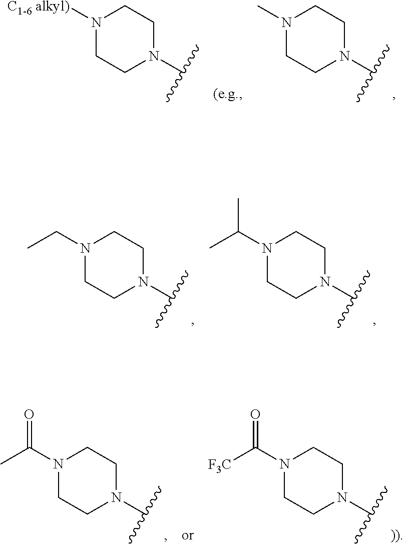

In certain embodiments, R^{A} is substituted or unsubstituted pyrrolidinyl (e.g., substituted or unsubstituted 3-pyrrolidinyl,

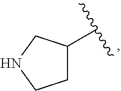 [SMILES: *C1CCNC1]

or (substituted or unsubstituted,

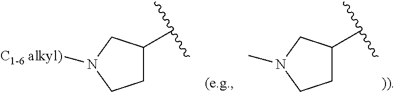

In certain embodiments, R^{A} is substituted or unsubstituted, 5- to 13-membered, bicyclic heterocyclyl. In certain embodiments, R^{A} is substituted or unsubstituted, 5- to 13-membered, bicyclic heterocyclyl that is fused, spiro, or bridged.

In certain embodiments, R^{A} is substituted or unsubstituted, 6- to 11-membered, monocyclic or bicyclic aryl. In certain embodiments, R^{A} is substituted or unsubstituted phenyl. In certain embodiments, R^{A} is Ph. In certain embodiments, R^{A} is substituted phenyl. In certain embodiments, R^{A} is ortho-substituted phenyl, meta-substituted phenyl, para-substituted phenyl, ortho, ortho-substituted phenyl, ortho, meta-substituted phenyl, ortho, para-substituted phenyl, meta, meta-substituted phenyl, or meta, para-substituted phenyl. In certain embodiments, R^{A} is of the formula:

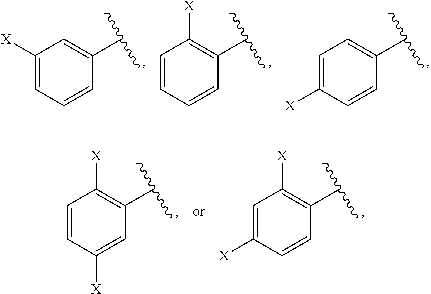

wherein each instance of X is independently hydrogen, halogen, substituted or unsubstituted, C₁₋₆alkyl, —OR^{a}, —N(R^{a})₂, —SR^{a}, or —CN. In certain embodiments, R^{A} is of the formula:

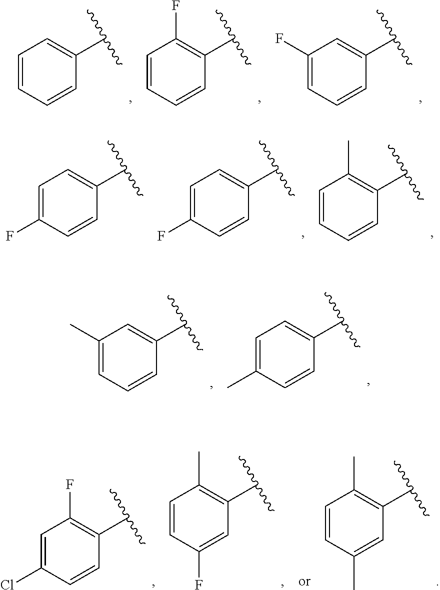

In certain embodiments, R^{A} is of the formula:

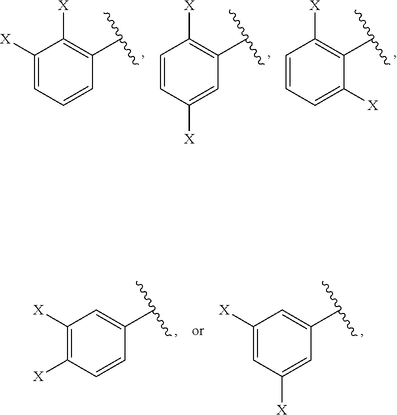

wherein each instance of X is independently hydrogen, halogen, substituted or unsubstituted, C₁₋₆ alkyl, —OR^{a}, —N(R^{a})₂, —SR^{a}, or —CN.

In certain embodiments, R^{A} is substituted or unsubstituted, 7- to 11-membered, bicyclic aryl. In certain embodiments, R^{A} is substituted or unsubstituted phenyl fused with substituted or unsubstituted, 3- to 7-membered, monocyclic carbocyclyl. In certain embodiments. R^{A} is substituted or unsubstituted phenyl fused with substituted or unsubstituted, 3- to 7-membered, monocyclic heterocyclyl. In certain embodiments, R is substituted or unsubstituted naphthyl. In certain embodiments, R^{A} is substituted or unsubstituted phenyl fused with substituted or unsubstituted, 5- or 6-membered, monocyclic heteroaryl.

In certain embodiments, R^{A} is substituted or unsubstituted, 5- to 11-membered, monocyclic or bicyclic heteroaryl. In certain embodiments, R^{A} is substituted or unsubstituted, 5-membered, monocyclic heteroaryl. In certain embodiments, R^{A} is substituted or unsubstituted, 6-membered, monocyclic heteroaryl. In certain embodiments, R^{A} is substituted or unsubstituted pyridinyl. In certain embodiments, R^{A} is substituted or unsubstituted, 6- to 11-membered, bicyclic heteroaryl. In certain embodiments, R^{A} is substituted or unsubstituted, 5- or 6-membered, monocyclic heteroaryl fused with substituted or unsubstituted, 3- to 7-membered, monocyclic carbocyclyl, or with substituted or unsubstituted, 3- to 7-membered, monocyclic heterocyclyl. In certain embodiments, R^{A} is substituted or unsubstituted, 5- or 6-membered, monocyclic heteroaryl fused with substituted or unsubstituted phenyl. In certain embodiments, R^{A} is substituted or unsubstituted, 5- or 6-membered, monocyclic heteroaryl fused with another substituted or unsubstituted, 5- or 6-membered, monocyclic heteroaryl.

In certain embodiments, when Z is a bond or C(R^{F})₂. R^{A} is —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂. In certain embodiments, R^{A} is —OR^{a} (e.g., —OH, —O(substituted or unsubstituted C₁₋₆ alkyl) (e.g., —OMe, —OCF₃, —OEt, —OPr, —OBu, or —OBn), or —O(substituted or unsubstituted phenyl) (e.g., —OPh)). In certain embodiments, R^{A} is —OMe. In certain embodiments, R^{A} is —SR^{a} (e.g., —SH, —S(substituted or unsubstituted C₁₋₆ alkyl) (e.g., —SMe, —SCF₃, —SEt, —SPr, —SBu, or —SBn), or —S(substituted or unsubstituted phenyl) (e.g., —SPh)). In certain embodiments, R^{A} is —N(R^{a})₂ (e.g., —NH₂, —NH (substituted or unsubstituted C₁₋₆ alkyl) (e.g., —NHMe), or —N (substituted or unsubstituted C₁₋₆ alkyl)-(substituted or unsubstituted C₁₋₆ alkyl) (e.g., —NMe₂)). In certain embodiments, R^{A} is —CN or —SCN. In certain embodiments, R^{A} is —NO₂. In certain embodiments, R^{A} is —C(═NR^{a})R^{a}, —C(═NR^{a})OR^{a}, or —C(═NR^{a})N(R^{a})₂. In certain embodiments, R^{A} is —C(═O)R^{a} (e.g., —C(═O)(substituted or unsubstituted alkyl) (e.g., —C(═O)Me) or —C(═O)(substituted or unsubstituted phenyl)). In certain embodiments, R^{A} is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, R^{A} is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl). —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R^{A} is —NR^{a}C(═O)R^{a} (e.g., NHC(═O)(substituted or unsubstituted C₁₋₆ alkyl) (e.g., —NHC(═O)Me) or —NHC(═O)(substituted or unsubstituted phenyl)). In certain embodiments, R^{A} is —NR^{a}C(═O)OR^{a}. In certain embodiments, R^{A} is —NR^{a}C(═O)N(R^{a})₂ (e.g., —NHC(═O)NH₂, —NHC(═O)NH(substituted or unsubstituted C₁₋₆ alkyl) (e.g., —NHC(═O)NHMe)). In certain embodiments, R^{A} is —OC(═O)R^{a} (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)).

In some embodiments, at least one R^{a} is hydrogen. In some embodiments, each R^{a} is hydrogen. In some embodiments, at least one R^{a} is not hydrogen. In some embodiments, each R^{a} is not hydrogen. In some embodiments, at least one R^{a} is substituted or unsubstituted C₁₋₆ alkyl. In certain embodiments, at least one R^{a} is substituted or unsubstituted C₁₋₄ alkyl. In certain embodiments, at least one R^{a} is substituted or unsubstituted C₅₋₆ alkyl. In certain embodiments, at least one R^{a} is Me. In certain embodiments, at least one R^{a} is Et. In certain embodiments, at least one R^{a} is Pr or Bu. In certain embodiments, at least one R^{a} is substituted methyl (e.g., fluorinated methyl). In certain embodiments, at least one R^{a} is —CH₂F, —CHF₂, or —CF₃. In certain embodiments, at least one R^{a} is substituted ethyl (e.g., fluorinated ethyl). In certain embodiments, at least one R^{a} is —CH₂CH₂F, —CH₂CHF₂, or —CH₂CF₃. In certain embodiments, at least one R^{a} is substituted propyl or substituted butyl (e.g., fluorinated propyl or fluorinated butyl).

In certain embodiments, at least one R^{a} is substituted or unsubstituted C₂₋₆ alkenyl. In certain embodiments, at least one R^{a} is substituted or unsubstituted C₂₋₄ alkenyl. In certain embodiments, at least one R^{a} is substituted or unsubstituted C₅₋₆ alkenyl. In certain embodiments, at least one R^{a} is substituted or unsubstituted vinyl or substituted or unsubstituted allyl.

In certain embodiments, at least one R^{a} is substituted or unsubstituted C₂₋₆ alkynyl. In certain embodiments, at least one R^{a} is substituted or unsubstituted C₂₋₄ alkynyl. In certain embodiments, at least one R^{a} is substituted or unsubstituted C₅₋₆ alkynyl. In certain embodiments, at least one R^{a} is substituted or unsubstituted ethynyl.

In certain embodiments, at least one R^{a} is substituted or unsubstituted, 3- to 7-membered, monocyclic carbocyclyl. In certain embodiments, at least one R^{a} is substituted or unsubstituted cyclopropyl. In certain embodiments, at least one R^{a} is substituted or unsubstituted cyclobutyl, substituted or unsubstituted cyclopentyl, or substituted or unsubstituted cyclohexyl. In certain embodiments, at least one R^{a} is substituted or unsubstituted, 3- to 7-membered, monocyclic heterocyclyl. In certain embodiments, at least one R^{a} is substituted or unsubstituted phenyl. In certain embodiments, at least one R^{a} is substituted or unsubstituted, 5- or 6-membered, monocyclic heteroaryl. In certain embodiments, at least one R^{a} is a nitrogen protecting group when attached to a nitrogen atom. In certain embodiments, at least one R^{a} is an oxygen protecting group when attached to an oxygen atom. In certain embodiments, at least one R^{a} is a sulfur protecting group when attached to a sulfur atom. In certain embodiments, two R^{a} groups attached to the same nitrogen atom are joined to form substituted or unsubstituted, 3- to 7-membered, monocyclic heterocyclyl. In certain embodiments, two R^{a} groups attached to the same nitrogen atom are joined to form substituted or unsubstituted, 5- or 6-membered, monocyclic heteroaryl.

In certain embodiments, —Z—R^{A} is hydrogen, halogen, or substituted or unsubstituted, C₁₋₁₂ alkyl. In certain embodiments, —Z—R^{A} is hydrogen.

In certain embodiments, R^{B} is hydrogen, halogen, or substituted or unsubstituted, C₁₋₁₂ alkyl. In certain embodiments, R^{B} is hydrogen. In certain embodiments, R^{B} is halogen or substituted or unsubstituted, C₁ alkyl. In certain embodiments, R^{B} is halogen (e.g., F, Cl, or Br). In certain embodiments, R^{B} is substituted or unsubstituted, C₁₋₁₂ alkyl (e.g., substituted or unsubstituted, C₁₋₆ alkyl). In certain embodiments, R^{B} is C₁ alkyl substituted with one or more halogen (e.g., F). In certain embodiments. R^{B} is unsubstituted C₁₋₆ alkyl (e.g., Me). In certain embodiments, R^{B} is substituted or unsubstituted, C₂₋₁₂ alkenyl (e.g., substituted or unsubstituted, C₂₋₆ alkenyl) or substituted or unsubstituted, C₂₋₁₂ alkynyl (e.g., substituted or unsubstituted, C₂₋₆ alkynyl). In certain embodiments, R^{B} is substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl.

In certain embodiments, R^{B} is substituted or unsubstituted, 3- to 13-membered, monocyclic or bicyclic carbocyclyl. In certain embodiments, R^{B} is substituted or unsubstituted, 3- to 7-membered, monocyclic carbocyclyl. In certain embodiments. R^{B} is substituted or unsubstituted cyclopropyl. In certain embodiments, R^{B} is substituted or unsubstituted cyclobutyl, substituted or unsubstituted cyclopentyl, or substituted or unsubstituted cyclohexyl. In certain embodiments, R^{B} is substituted or unsubstituted, 5- to 13-membered, bicyclic carbocyclyl. In certain embodiments, R^{B} is substituted or unsubstituted, 5- to 13-membered, bicyclic carbocyclyl that is fused, spiro, or bridged.

In certain embodiments, R^{B} is substituted or unsubstituted, 3- to 13-membered, monocyclic or bicyclic heterocyclyl. In certain embodiments, R^{B} is substituted or unsubstituted, 3- to 7-membered, monocyclic heterocyclyl. In certain embodiments, R^{B} is substituted or unsubstituted, 5-membered, monocyclic heterocyclyl. In certain embodiments, R^{B} is substituted or unsubstituted, 6-membered, monocyclic heterocyclyl. In certain embodiments, R^{B} is substituted or unsubstituted oxetanyl, substituted or unsubstituted azetidinyl, substituted or unsubstituted tetrahydrofuranyl, substituted or unsubstituted pyrrolidinyl, substituted or unsubstituted tetrahydropyranyl, substituted or unsubstituted piperidinyl, substituted or unsubstituted morpholinyl, or substituted or unsubstituted piperazinyl. In certain embodiments, R^{B} is substituted or unsubstituted piperazinyl (e.g., substituted or unsubstituted 1-piperazinyl,

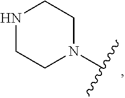 [SMILES: *N1CCNCC1]

or
(substituted or unsubstituted,

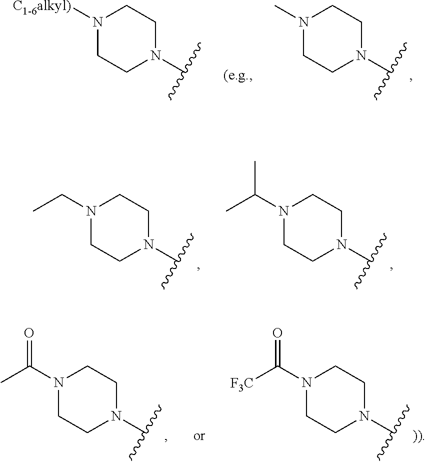

In certain embodiments, R^{B} is substituted or unsubstituted pyrrolidinyl (e.g., substituted or unsubstituted 3-pyrrolidinyl,

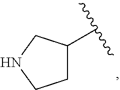 [SMILES: *C1CCNC1]

or
(substituted or unsubstituted,

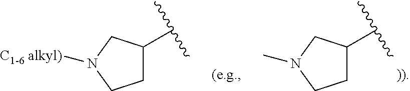

In certain embodiments, R^{B} is substituted or unsubstituted, 5- to 13-membered, bicyclic heterocyclyl. In certain embodiments, R^{B} is substituted or unsubstituted, 5- to 13-membered, bicyclic heterocyclyl that is fused, spiro, or bridged.

In certain embodiments, R^{B} is substituted or unsubstituted, 6- to 11-membered, monocyclic or bicyclic aryl. In certain embodiments, R^{B} is substituted or unsubstituted phenyl. In certain embodiments, R^{B} is Ph. In certain embodiments, R^{B} is substituted phenyl. In certain embodiments, R^{B} is ortho-substituted phenyl, meta-substituted phenyl, para-substituted phenyl, ortho, ortho-substituted phenyl, ortho, meta-substituted phenyl, ortho, para-substituted phenyl, meta, meta-substituted phenyl, or meta, para-substituted phenyl. In certain embodiments, R^{B} is of the formula:

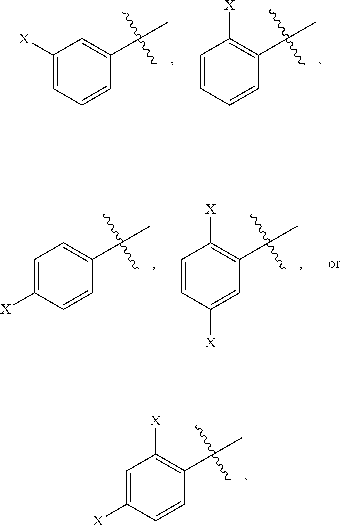

wherein each instance of X is independently hydrogen, halogen, substituted or unsubstituted, C₁₋₆ alkyl, —OR^{a}, —N(R^{a})₂, —SR^{a}, or —CN. In certain embodiments, R^{B} is of the formula

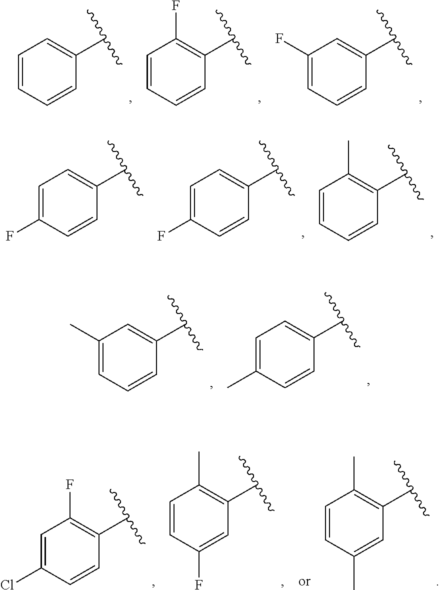

In certain embodiments, R^{B} is of the formula:

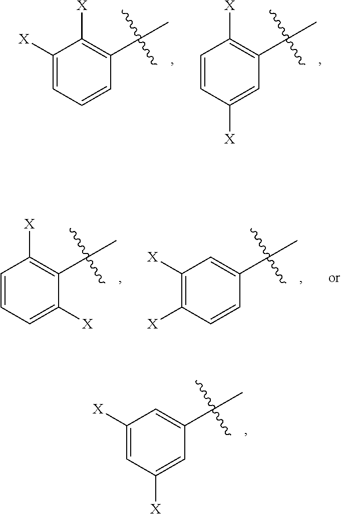

herein each instance of X is independently hydrogen, halogen, substituted or unsubstituted, C₁₋₆ alkyl, —OR^{a}, —N(R^{a})₂, —SR^{a}, or —CN.

In certain embodiments, R^{B} is substituted or unsubstituted, 7- to 11-membered, bicyclic aryl. In certain embodiments, R^{B} is substituted or unsubstituted phenyl fused with substituted or unsubstituted, 3- to 7-membered, monocyclic carbocyclyl. In certain embodiments. R^{B} is substituted or unsubstituted phenyl fused with substituted or unsubstituted, 3- to 7-membered, monocyclic heterocyclyl. In certain embodiments, R^{B} is substituted or unsubstituted naphthyl. In certain embodiments, R^{B} is substituted or unsubstituted phenyl fused with substituted or unsubstituted, 5- or 6-membered, monocyclic heteroaryl. In certain embodiments, R^{B} is substituted or unsubstituted indolyl (e.g., 5-indolyl).

In certain embodiments, R^{B} is substituted or unsubstituted, 5- to 11-membered, monocyclic or bicyclic heteroaryl. In certain embodiments, R^{B} is substituted or unsubstituted, 5-membered, monocyclic heteroaryl. In certain embodiments. R^{B} is substituted or unsubstituted, 6-membered, monocyclic heteroaryl. In certain embodiments, R^{B} is substituted or unsubstituted pyridinyl (e.g., 3-pyridinyl). In certain embodiments. R^{B} is substituted or unsubstituted pyrazolyl (e.g., 4-pyrazolyl). In certain embodiments, R^{B} is substituted or unsubstituted, 6- to 11-membered, bicyclic heteroaryl. In certain embodiments, R^{B} is substituted or unsubstituted indolyl. In certain embodiments, R^{B} is substituted or unsubstituted, 5- or 6-membered, monocyclic heteroaryl fused with substituted or unsubstituted, 3- to 7-membered, monocyclic carbocyclyl, or with substituted or unsubstituted, 3- to 7-membered, monocyclic heterocyclyl. In certain embodiments. R^{B} is substituted or unsubstituted, 5- or 6-membered, monocyclic heteroaryl fused with substituted or unsubstituted phenyl. In certain embodiments, R^{B} is substituted or unsubstituted, 5- or 6-membered, monocyclic heteroaryl fused with another substituted or unsubstituted, 5- or 6-membered, monocyclic heteroaryl.

In certain embodiments, R^{B} is —OR^{a}, —N(R^{a})₂, —SR^{a}, —CN, —SCN, —C(═NR^{a})R^{a}, —C(═NR^{a})OR^{a}, —C(═NR)N(R)₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R′)₂. In certain embodiments, R^{B} is —OR^{a} (e.g., —OH, —O(substituted or unsubstituted C₁₋₆ alkyl)(e.g., —OMe, —OCF₃, —OEt, —OPr, —OBu, or —OBn), or —O(substituted or unsubstituted phenyl) (e.g., —OPh)). In certain embodiments, R^{B} is —OMe. In certain embodiments. R^{B} is —SR^{a} (e.g., —SH, —S(substituted or unsubstituted C₁₋₆ alkyl) (e.g., —SMe, —SCF₃, —SEt, —SPr, —SBu, or —SBn), or —S(substituted or unsubstituted phenyl) (e.g., —SPh)). In certain embodiments, R^{B} is —N(R^{a})₂ (e.g., —NH₂, —NH(substituted or unsubstituted C₁₋₆ alkyl) (e.g., —NHMe), or —N(substituted or unsubstituted C₁₋₆ alkyl)-(substituted or unsubstituted C₁₋₆ alkyl) (e.g., —NMe₂)). In certain embodiments, R^{B} is —CN or —SCN. In certain embodiments, R^{B} is —NO₂. In certain embodiments, R^{B} is —C(═NR^{a})R^{a}, —C(═NR^{a})OR^{a}, or —C(═NR^{a})N(R^{a})₂. In certain embodiments, R^{B} is —C(═O)R^{a}(e.g., —C(═O)(substituted or unsubstituted alkyl) (e.g., —C(═O)Me) or —C(═O)(substituted or unsubstituted phenyl)). In certain embodiments, R^{B} is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, R^{B} is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R^{B} is —NR^{a}C(═O)R^{a}(e.g., —NHC(═O)(substituted or unsubstituted C₁₋₆ alkyl) (e.g., —NHC(═O)Me) or —NHC(═O)(substituted or unsubstituted phenyl)). In certain embodiments, R^{B} is —NR^{a}C(═O)OR^{a}. In certain embodiments, R^{B} is —NR^{a}C(═O)N(R^{a})₂ (e.g., —NHC(═O)NH₂, —NHC(═O)NH(substituted or unsubstituted C₁a alkyl) (e.g., —NHC(═O)NHMe)). In certain embodiments, R^{B} is —OC(═O)R (e.g., —OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R^{a})₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)).

In certain embodiments, R^{A} and R^{B} are joined to form substituted or unsubstituted, 3- to 7-membered, monocyclic carbocyclic ring, substituted or unsubstituted, 3- to 7-membered, monocyclic heterocyclic ring, substituted or unsubstituted, phenyl ring, or substituted or unsubstituted, 5- or 6-membered, monocyclic heteroaryl ring. In certain embodiments, R^{A} and R^{B} are joined to form substituted or unsubstituted, cyclohexyl or cyclohexenyl.

In certain embodiments, at least one of R^{A} and R^{B} is substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl.

In certain embodiments, Y is C(R^{N})₂ (e.g., CH₂). In certain embodiments, Y is O. In certain embodiments, Y is S. In certain embodiments, Y is NR^{G} (e.g., NH).

In certain embodiments, each instance of R^{N} is hydrogen. In certain embodiments, at least one instance of R^{N} is halogen (e.g., F) or substituted or unsubstituted, C₁ alkyl (e.g., Me).

In certain embodiments, R^{G} is hydrogen, or substituted or unsubstituted C₁₋₆ alkyl. In certain embodiments, R^{G} is hydrogen. In certain embodiments, R^{G} is unsubstituted C₁₋₆ alkyl (e.g., Me). In certain embodiments, R^{G} is a nitrogen protecting group.

In certain embodiments, R^{C} is hydrogen, or substituted or unsubstituted C₁₋₆ alkyl. In certain embodiments, R^{C} is hydrogen. In certain embodiments, R^{C} is unsubstituted C₁₋₆ alkyl (e.g., Me). In certain embodiments, R^{C} is a nitrogen protecting group when attached to a nitrogen group, an oxygen protecting group when attached to an oxygen atom, or a sulfur protecting group when attached to a sulfur atom.

In certain embodiments, R^{C} and R^{E} are joined to form a substituted or unsubstituted, 3- to 7-membered (e.g., 6-membered), monocyclic heterocyclic ring. In certain embodiments, R^{C} and R^{E} are joined to form a substituted or unsubstituted 1,2,3,4-tetrahydropyrazinyl ring.

In certain embodiments, R^{C} and one instance of R^{F} are joined to form a substituted or unsubstituted, 3- to 7-membered (e.g., 6-membered), monocyclic heterocyclic ring. In certain embodiments, R^{C} and one instance of R^{F} are joined to form a substituted or unsubstituted 1,2,3,4-tetrahydropyrazinyl ring.

In certain embodiments, X^{A} is N. In certain embodiments. In certain embodiments, X^{A} is NR^{H} (e.g., NH).

In certain embodiments, R^{H} is hydrogen. In certain embodiments, R^{H} is substituted or unsubstituted, C₁₋₆ alkyl (e.g., Me). In certain embodiments. R^{H} is a nitrogen protecting group.

In certain embodiments, X^{B} is N. In certain embodiments, X^{B} is NR (e.g., NH). In certain embodiments, X^{B} is CR^{J}(e.g., CH).

In certain embodiments, R^{M} is hydrogen. In certain embodiments, R^{M} is substituted or unsubstituted, C₁₋₆, alkyl (e.g, Me). In certain embodiments, R^{M} is a nitrogen protecting group.

In certain embodiments, R^{J} is hydrogen or halogen. In certain embodiments, R^{J} is hydrogen. In certain embodiments, R^{J} is halogen (e.g., F, Cl, or Br). In certain embodiments, R^{J} is substituted or unsubstituted, C₁₋₄ alkyl (e.g, Me).

In certain embodiments. X^{C} is N. In certain embodiments, X^{C} is CR^{L} (e.g., CH).

In certain embodiments, R^{L} is hydrogen. In certain embodiments, R^{L} is halogen (e.g., F). In certain embodiments, R^{L} is substituted or unsubstituted, C₁₋₆ alkyl (e.g. Me).

In certain embodiments, X^{D} is N. In certain embodiments, X^{D} is C.

In certain embodiments, —U—V— is —C(═O)—NR^{K}—. In certain embodiments, —U—V— is —C(═O)—NH—. In certain embodiments, —U—V— is —C(═O)—NMe-. In certain embodiments, —U—V— is —NR^{K}—C(═O)— (e.g., —U—V— is —NH—C(═O— or —U—V— is —NMe-C(═O)—).

In certain embodiments, R^{K} is hydrogen. In certain embodiments, R^{K} is substituted or unsubstituted, C₁₋₆ alkyl (e.g, Me). In certain embodiments, R^{K} is a nitrogen protecting group.

In certain embodiments, L is a bond. In certain embodiments, L is substituted or unsubstituted, C₁ alkylene (e.g., unsubstituted C₁ alkylene). In certain embodiments, L is is —CH₂—, —CH₂—CH—, or —(CH₂)—. In certain embodiments, L is substituted or unsubstituted, C₂₋₆ alkenylene, or substituted or unsubstituted, C₂₋₆ alkynylene.

In certain embodiments, R^{D} is hydrogen. In certain embodiments, R^{D} is.

In certain embodiments, R^{D} is substituted or unsubstituted, C₁₋₁₂ alkyl (e.g, substituted or unsubstituted, C₁₋₆ alkyl). In certain embodiments, R^{D} is substituted or unsubstituted, C₂₋₁₂ alkenyl (e.g, substituted or unsubstituted, C₂₋₆ alkenyl) or substituted or unsubstituted, C₂₋₁₂ alkynyl (e.g., substituted or unsubstituted, C₂₋₆ alkynyl).

In certain embodiments, R^{D} is substituted or unsubstituted, 3- to 13-membered, monocyclic or bicyclic carbocyclyl. In certain embodiments, R^{D} is substituted or unsubstituted, 3- to 7-membered, monocyclic carbocyclyl. In certain embodiments, R^{D} is substituted or unsubstituted cyclopropyl. In certain embodiments, R^{D} is substituted or unsubstituted cyclobutyl, substituted or unsubstituted cyclopentyl, or substituted or unsubstituted cyclohexyl. In certain embodiments. R^{D} is substituted or unsubstituted, 5- to 13-membered, bicyclic carbocyclyl. In certain embodiments, R^{D} is substituted or unsubstituted, 5- to 13-membered, bicyclic carbocyclyl that is fused, spiro, or bridged.

In certain embodiments, R^{D} is substituted or unsubstituted, 3- to 13-membered, monocyclic or bicyclic heterocyclyl. In certain embodiments, R^{D} is substituted or unsubstituted, 3- to 7-membered (e.g., 6-membered), monocyclic heterocyclyl. In certain embodiments, R^{D} is substituted or unsubstituted oxetanyl, substituted or unsubstituted azetidinyl, substituted or unsubstituted tetrahydrofuranyl, substituted or unsubstituted pyrrolidinyl, substituted or unsubstituted tetrahydropyranyl, substituted or unsubstituted piperidinyl, substituted or unsubstituted morpholinyl, or substituted or unsubstituted piperazinyl. In certain embodiments, R^{D} is substituted or unsubstituted piperazinyl (e.g., substituted or unsubstituted 1-piperazinyl). In certain embodiments, R^{D} is substituted or unsubstituted piperidinyl (e.g., substituted or unsubstituted 4-piperidinyl). In certain embodiments, R^{D} is substituted or unsubstituted, 5- to 13-membered, bicyclic heterocyclyl. In certain embodiments, R^{D} is substituted or unsubstituted, 5- to 13-membered, bicyclic heterocyclyl that is fused, spiro, or bridged.

In certain embodiments, R^{D} is substituted or unsubstituted, 6- to 11-membered, monocyclic or bicyclic aryl. In certain embodiments, R^{D} is substituted or unsubstituted phenyl. In certain embodiments, R^{D} is Ph. In certain embodiments, R^{D} is substituted phenyl. In certain embodiments, R^{D} is ortho-substituted phenyl, meta-substituted phenyl, para-substituted phenyl, ortho, ortho-substituted phenyl, ortho, meta-substituted phenyl, ortho, para-substituted phenyl, meta, meta-substituted phenyl, or meta, para-substituted phenyl. In certain embodiments, R^{D} is of the formula:

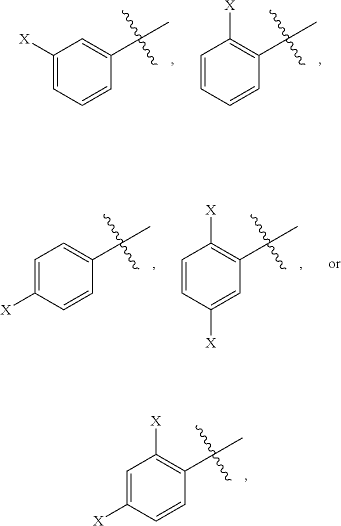

wherein each instance of X is independently hydrogen, halogen, substituted or unsubstituted, C₁₋₆ alkyl, —OR^{a}, —N(R^{a})₂, —SR^{a} or —CN. In certain embodiments, R^{D} is of the formula:

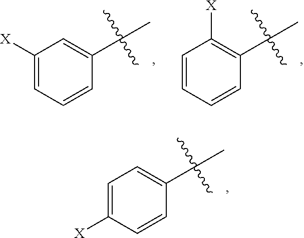

wherein X is —C(═O)OR^{a} (e.g., —C(═O)OH). In certain embodiments, R^{D} is of the formula:

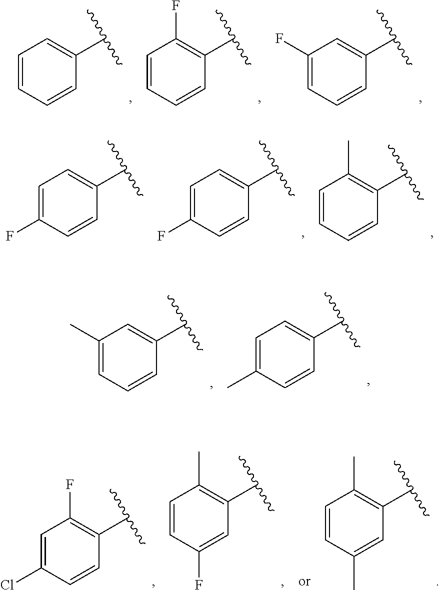

In certain embodiments, R^{D} is of the formula:

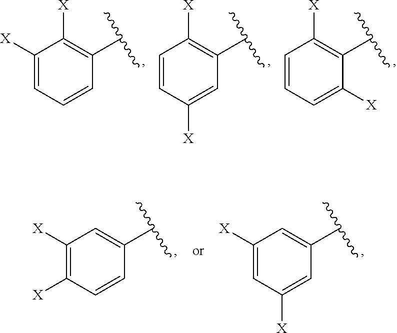

wherein each instance of X is independently hydrogen, halogen, substituted or unsubstituted, C₁₋₆ alkyl, —OR^{a}, N(R^{a})₂, —SR^{a}, or —CN.

In certain embodiments, R^{D} is substituted or unsubstituted, 7- to 11-membered, bicyclic aryl. In certain embodiments, R^{D} is substituted or unsubstituted phenyl fused with substituted or unsubstituted, 3- to 7-membered, monocyclic carbocyclyl. In certain embodiments, R^{D} is substituted or unsubstituted phenyl fused with substituted or unsubstituted, 3- to 7-membered, monocyclic heterocyclyl. In certain embodiments, R^{D} is substituted or unsubstituted naphthyl. In certain embodiments, R^{D} is substituted or unsubstituted phenyl fused with substituted or unsubstituted, 5- or 6-membered, monocyclic heteroaryl.

In certain embodiments, R^{D} is substituted or unsubstituted, 5- to 11-membered, monocyclic or bicyclic heteroaryl. In certain embodiments, R^{D} is substituted or unsubstituted, 5-membered, monocyclic heteroaryl. In certain embodiments, R^{D} is substituted or unsubstituted, 6-membered, monocyclic heteroaryl. In certain embodiments, R^{D} is substituted or unsubstituted pyridinyl. In certain embodiments, R^{D} is substituted or unsubstituted, 6- to 11-membered, bicyclic heteroaryl. In certain embodiments, R^{D} is substituted or unsubstituted, 5- or 6-membered, monocyclic heteroaryl fused with substituted or unsubstituted, 3- to 7-membered, monocyclic carbocyclyl, or with substituted or unsubstituted, 3- to 7-membered, monocyclic heterocyclyl. In certain embodiments, R^{D} is substituted or unsubstituted, 5- or 6-membered, monocyclic heteroaryl fused with substituted or unsubstituted phenyl. In certain embodiments, R^{D} is substituted or unsubstituted, 5- or 6-membered, monocyclic heteroaryl fused with another substituted or unsubstituted, 5- or 6-membered, monocyclic heteroaryl.

In certain embodiments, R^{D} is —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a}), —NO₂, —NR^{a}C(═O)R^{a}, —NR^{a}C(═O)OR^{a}, —NR^{a}C(═O)N(R)₂, —OC(═O)R^{a}, —OC(═O)OR^{a}, or —OC(═O)N(R^{a})₂. In certain embodiments, R^{D} is —OR^{a} (e.g., —OH, —O(substituted or unsubstituted C₁₋₆ alkyl)(e.g., —OMe, —OCF₃, —OEt, —OPr, —OBu, or —OBn), or —O(substituted or unsubstituted phenyl) (e.g., —OPh)). In certain embodiments, R^{D} is-OMe. In certain embodiments, R^{D} is —SR^{a} (e.g., —SH, —S(substituted or unsubstituted C₁₋₆ alkyl) (e.g., —SMe, —SCF₃, —SEt, —SPr, —SBu, or —SBn), or —S(substituted or unsubstituted phenyl) (e.g., —SPh)). In certain embodiments, R^{D} is —N(R)₂ (e.g., —NH₂, —NH(substituted or unsubstituted C₁₋₆ alkyl) (e.g., —NHMe), or —N(substituted or unsubstituted C₁₋₆ alkyl)-(substituted or unsubstituted C₁₋₆ alkyl) (e.g., —NMe₂)). In certain embodiments, R^{D} is —CN or —SCN. In certain embodiments, R^{D} is —NO₂. In certain embodiments, R^{D} is —C(═NR^{a})R^{a}, —C(═NR^{a})OR^{a}, or —C(═NR^{a})N(R^{a})₂. In certain embodiments, R^{D} is —C(═O)R^{a} (e.g., —C(═O)(substituted or unsubstituted alkyl) (e.g., —C(═O)Me) or —C(═O)(substituted or unsubstituted phenyl)). In certain embodiments. R^{D} is —C(═O)OR^{a} (e.g., —C(═O)OH, —C(═O)O(substituted or unsubstituted alkyl) (e.g., —C(═O)OMe), or —C(═O)O(substituted or unsubstituted phenyl)). In certain embodiments, R^{D} is —C(═O)N(R^{a})₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-substituted or unsubstituted alkyl)). In certain embodiments, R^{D} is —NR^{a}C(═O)R^{a} (e.g., —NHC(═O)(substituted or unsubstituted C₁₋₆ alkyl) (e.g., —NHC(═O)Me) or —NHC(═O)(substituted or unsubstituted phenyl)). In certain embodiments, R^{D} is —NR^{a}C(═O)OR^{a}. In certain embodiments. R^{D} is —NR^{a}C(═O)N(R^{a})₂ (e.g., —NHC(═O)NH₂, —NHC(═O)NH(substituted or unsubstituted C₁₋₆ alkyl) (e.g., —NHC(═O)NHMe)). In certain embodiments, R^{D} is —OC(═O)R^{a} (e.g.—OC(═O)(substituted or unsubstituted alkyl) or —OC(═O)(substituted or unsubstituted phenyl)), —OC(═O)OR^{a} (e.g., —OC(═O)O(substituted or unsubstituted alkyl) or —OC(═O)O(substituted or unsubstituted phenyl)), or —OC(═O)N(R)₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)).

In certain embodiments, R^{D} is of the formula:

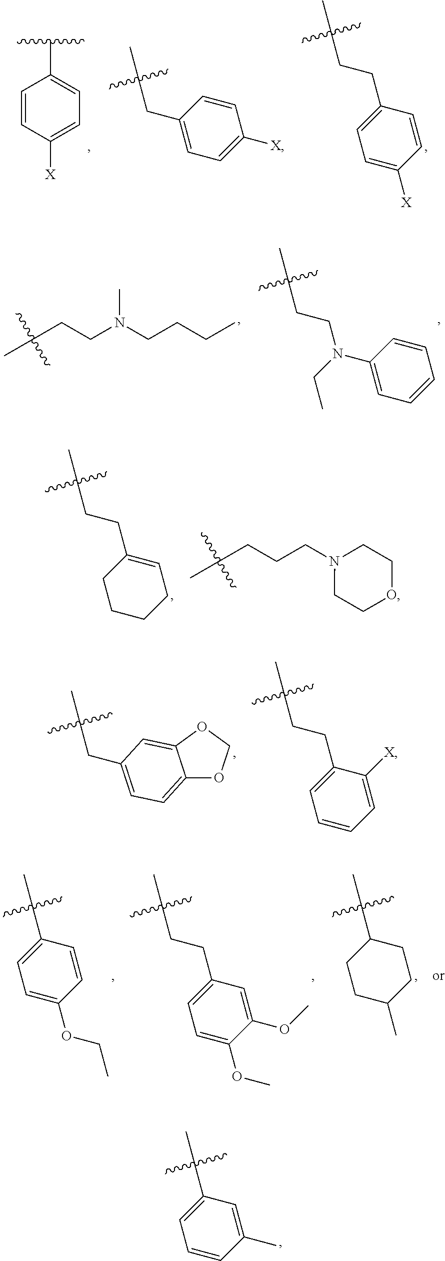

wherein X is hydrogen, halogen, substituted or unsubstituted, C₁₋₆alkyl, —OR^{a}, —N(R^{a})₂, —SR^{a}, or —CN.

In certain embodiments, R^{D} and R^{K} are joined to form a substituted or unsubstituted 3- to 7-membered, monocyclic heterocylic ring, or substituted or unsubstituted, 5- to 6-membered, monocyclic heteroaryl ring (e.g. substituted or unsubstituted piperidinyl ring).

In certain embodiments, the compound of Formula (I) is of the formula:

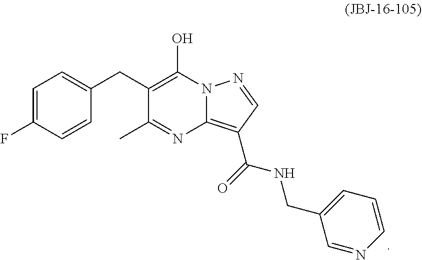 [SMILES: Cc1nc2c(C(=O)NCc3cccnc3)cnn2c(O)c1Cc1ccc(F)cc1]

In certain embodiments, the compound of Formula (I) is of the formula:

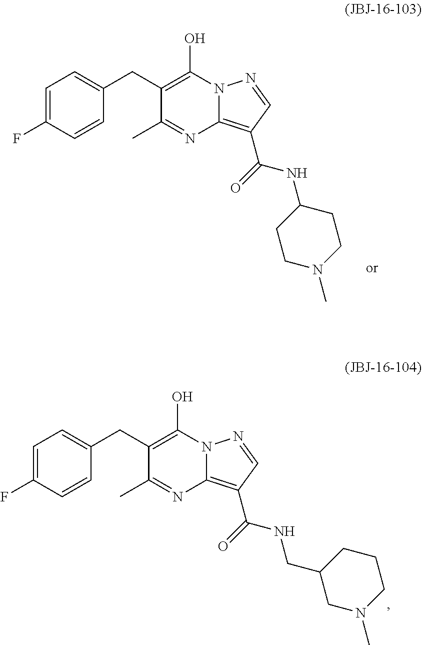 [SMILES: Cc1nc2c(C(=O)NC3CCN(C)CC3)cnn2c(O)c1Cc1ccc(F)cc1 ; Cc1nc2c(C(=O)NCC3CCCN(C)C3)cnn2c(O)c1Cc1ccc(F)cc1]

In certain embodiments, the compound of Formula (I) is of the formula:

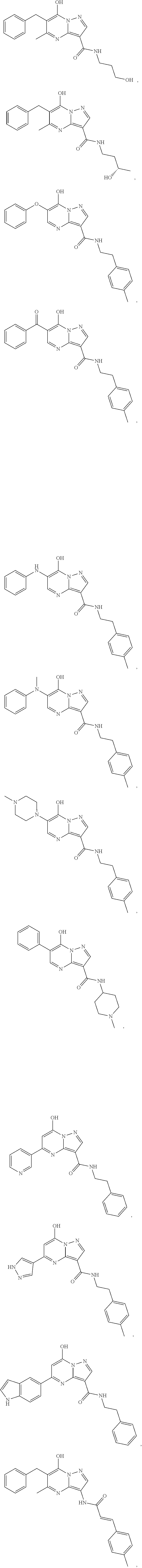

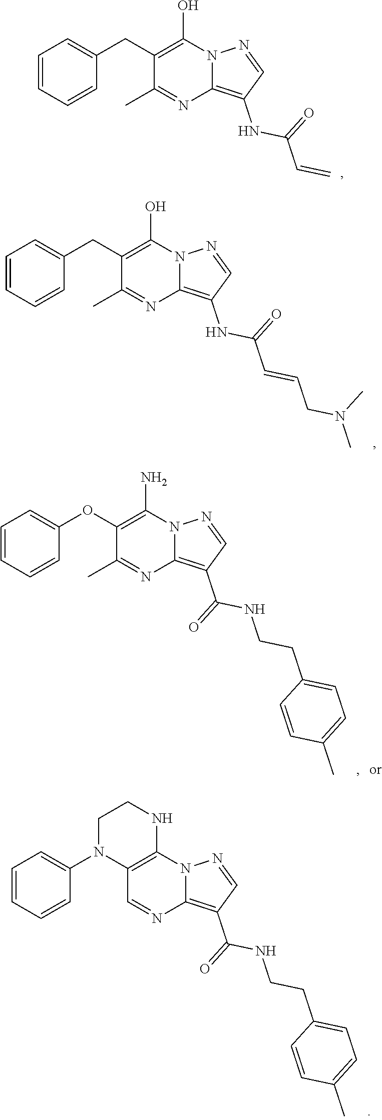 [SMILES: C=CC(=O)Nc1cnn2c(O)c(Cc3ccccc3)c(C)nc12 ; Cc1nc2c(NC(=O)/C=C/CN(C)C)cnn2c(O)c1Cc1ccccc1 ; Cc1ccc(CCNC(=O)c2cnn3c(N)c(Oc4ccccc4)c(C)nc23)cc1 ; Cc1ccc(CCNC(=O)c2cnn3c4c(cnc23)N(c2ccccc2)CCN4)cc1]

In certain embodiments, the compound of Formula (I) is of the formula:

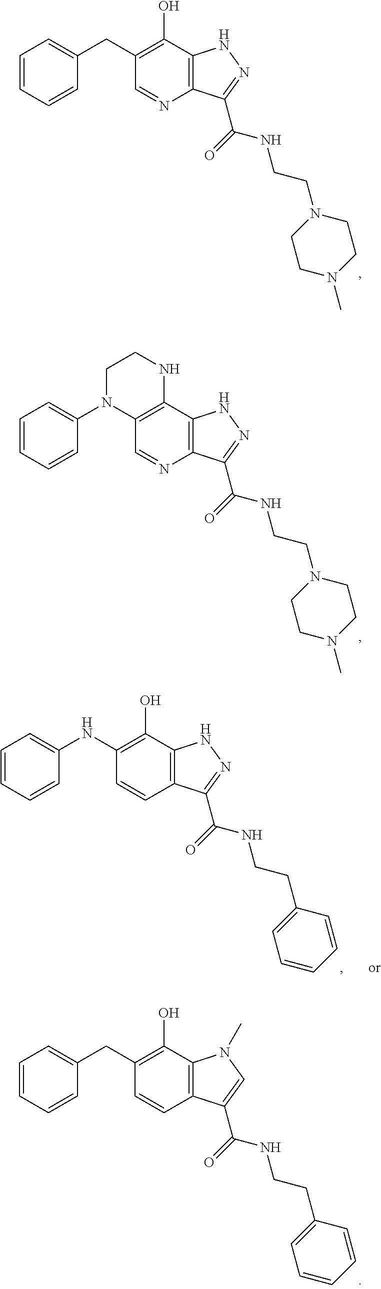 [SMILES: CN1CCN(CCNC(=O)c2n[nH]c3c(O)c(Cc4ccccc4)cnc23)CC1 ; CN1CCN(CCNC(=O)c2n[nH]c3c4c(cnc23)N(c2ccccc2)CCN4)CC1 ; O=C(NCCc1ccccc1)c1n[nH]c2c(O)c(Nc3ccccc3)ccc12 ; Cn1cc(C(=O)NCCc2ccccc2)c2ccc(Cc3ccccc3)c(O)c21]

In certain embodiments, the compound of Formula (I) is of the formula:

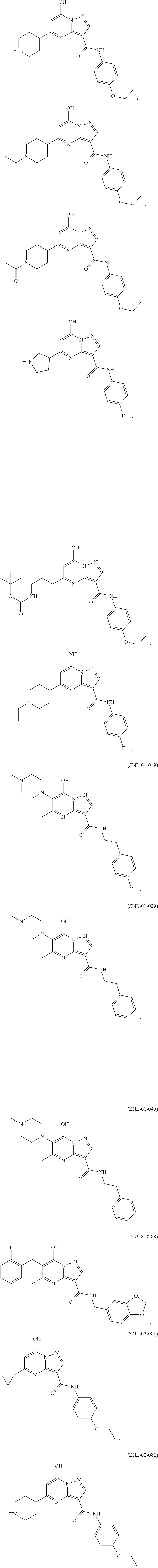

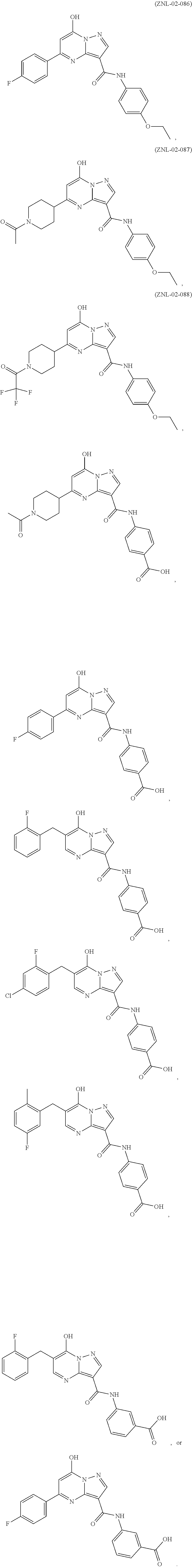

In certain embodiments, a compound described herein is a compound of Formula (I), or a pharmaceutically acceptable salt, solvate, hydrate, polymorph, co-crystal, tautomer, stereoisomer, isotopically labeled derivative, or prodrug thereof. In certain embodiments, a compound described herein is a compound of Formula (I), or a pharmaceutically acceptable salt, tautomer, or stereoisomer thereof. In certain embodiments, a compound described herein is a compound of Formula (I), or a pharmaceutically acceptable salt thereof

### Pharmaceutical Compositions and Kits

In another aspect, the present disclosure provides pharmaceutical compositions comprising:

a compound of any one of the preceding claims, or a pharmaceutically acceptable salt, solvate, hydrate, polymorph, co-crystal, tautomer, stereoisomer, isotopically labeled derivative, or prodrug thereof; and

optionally a pharmaceutically acceptable excipient.

In another aspect, the present disclosure provides kits comprising:

a compound of Formula (I), or a pharmaceutically acceptable salt, solvate, hydrate, polymorph, co-crystal, tautomer, stereoisomer, isotopically labeled derivative, or prodrug thereof, or a pharmaceutical composition described herein; and

instructions for using the compound, or a pharmaceutically acceptable salt, solvate, hydrate, polymorph, co-crystal, tautomer, stereoisomer, isotopically labeled derivative, or prodrug thereof, or the pharmaceutical composition.

The compounds of Formula (I), and pharmaceutically acceptable salts, solvates, hydrates, polymorphs, co-crystals, tautomers, stereoisomers, isotopically labeled derivatives, and prodrugs thereof; and compounds of any one of the formulae:

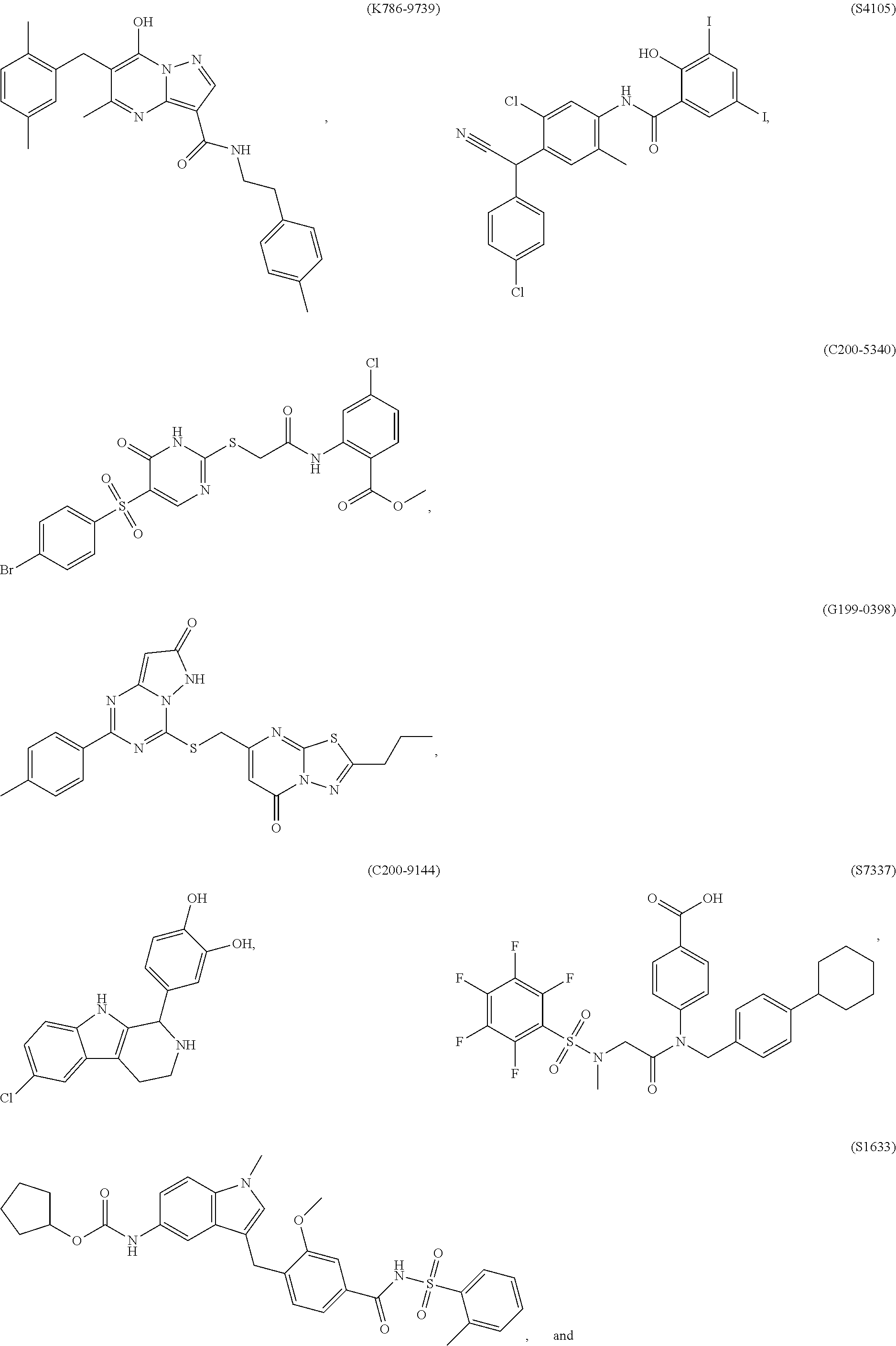

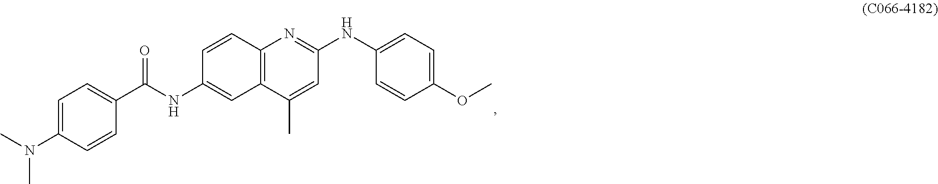

and pharmaceutically acceptable salts, solvates, hydrates, polymorphs, co-crystals, tautomers, stereoisomers, derivatives (e.g., isotopically labeled derivatives), and prodrugs thereof, may be antiviral agents.

Without being bound by any particular theory, the compounds described herein may target the prefusion form of the DENV envelope glycoprotein (E) and block viral entry by inhibiting membrane fusion. We used preliminary pyrimidine inhibitor as a probe to develop an efficient and reliable HTS assay by targeting the envelope glycoprotein to screen out more entry inhibitor candidates. We further show that this pharmacological approach is applicable against Dengue viruses by demonstrating inhibition of virus infection on BHK21 cells. Collectively, these findings expand the application for developing small molecule antivirals that can engage the envelope glycoprotein extracellularly to prevent Dengue infection.

The present disclosure provides pharmaceutical compositions comprising an antiviral agent, e.g., a compound of Formula (I), and pharmaceutically acceptable salts, solvates, hydrates, polymorphs, co-crystals, tautomers, stereoisomers, isotopically labeled derivatives, and prodrugs thereof, as described herein, and optionally a pharmaceutically acceptable excipient. In certain embodiments, the antiviral agent is provided in an effective amount in the pharmaceutical composition. In certain embodiments, the effective amount is a therapeutically effective amount. In certain embodiments, the effective amount is a prophylactically effective amount.

In certain embodiments, the effective amount is an amount effective for inhibiting the activity of a protein kinase by at least about 10%, at least about 20%, at least about 30%, at least about 40%, at least about 50%, at least about 60%, at least about 70%, at least about 80%, at least about 90%, at least about 95%, or at least about 98%.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers. (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathono®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate. D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline. Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein further comprises an additional pharmaceutical agent (e.g., antiviral agent). In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which are different from the compound or composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids. DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., viral infection). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include anti-proliferative agents, anti-cancer agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including surgery, radiation therapy, transplantation (e.g., stem cell transplantation, bone marrow transplantation), immunotherapy, and chemotherapy.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., viral infection) in a subject in need thereof. In certain embodiments, the kits are useful for preventing a disease (e.g., viral infection) in a subject in need thereof. In certain embodiments, the kits are useful for reducing the risk of developing a disease (e.g., viral infection) in a subject in need thereof. In certain embodiments, the kits are useful for inhibiting the activity (e.g., aberrant activity, such as increased activity) of a protein kinase in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., viral infection) in a subject in need thereof. In certain embodiments, the kits and instructions provide for preventing a disease (e.g., viral infection) in a subject in need thereof. In certain embodiments, the kits and instructions provide for reducing the risk of developing a disease (e.g., viral infection) in a subject in need thereof. In certain embodiments, the kits and instructions provide for inhibiting the activity (e.g., aberrant activity, such as increased activity) of a protein kinase in a subject or cell. A kit described herein may include one or more additional pharmaceutical agents described herein as a separate composition.

### Methods of Use

In another aspect, the present disclosure provides methods for the prevention and/or treatment of viral infections comprising administering to a subject in need thereof an effective amount of an antiviral agent or pharmaceutical composition described herein. In certain embodiments, the antiviral agent useful in the present disclosure is a compound of Formula (I), or a pharmaceutically acceptable salt, solvate, hydrate, polymorph, co-crystal, tautomer, stereoisomer, isotopically labeled derivative, or prodrug thereof. In certain embodiments, the antiviral agent useful in the present disclosure is compound K786-9739, S4105, C200-5340, G199-0398, C200-9144, S7337, S1633, or C066-4182, or a pharmaceutically acceptable salt, solvate, hydrate, polymorph, co-crystal, tautomer, stereoisomer, derivative (e.g., isotopically labeled derivative), or prodrug thereof. In certain embodiments, the antiviral agent useful in the present disclosure is compound C429-0385 or C218-0288, or a pharmaceutically acceptable salt, solvate, hydrate, polymorph, co-crystal, tautomer, stereoisomer, derivative (e.g., isotopically labeled derivative), or prodrug thereof.

In certain embodiments, the antiviral agent useful in the present disclosure is a combination of one or more compounds described herein. In certain embodiments, the antiviral agent useful in the present disclosure further includes an additional pharmaceutical agent (e.g., additional antiviral agent).

The present disclosure also provides methods of inhibiting the entry of a virus into a cell comprising contacting the cell with an effective amount of an antiviral agent or pharmaceutical composition described herein.

The present disclosure also provides methods of inhibiting an envelope glycoprotein of a virus comprising contacting the virus with an effective amount of an antiviral agent or pharmaceutical composition described herein.

The present disclosure also provides methods of inhibiting the fusion between the envelope of a virus and the membrane of a cell comprising contacting the virus or cell with an effective amount of an antiviral agent or pharmaceutical composition described herein.

The present disclosure also provides methods of reducing viral load comprising administering to a subject in need thereof an effective amount of an antiviral agent or pharmaceutical composition described herein. The antiviral agent or pharmaceutical composition described herein may be administered within 1 day, 2 days, 3 days, 4 days, 5 days, 6 days, 7 days, 2 weeks, 3 weeks, or 1 month of exposure to the virus. In certain embodiments, the time of viral clearance is reduced. In certain embodiments, morbidity or mortality of the subject, who may or may not have been infected with the virus or has been exposed to the virus, is reduced.

Viral load may be determined by measuring the titer or level of virus in a tissue or bodily fluid of the subject. Measuring the viral load can be accomplished by any conventional assay, such as ones described in the literature (see, e.g., Medical Microbiology; 3rd Ed.; Murray et al., eds.; Mosby, Inc.: Philadelphia, Pa., 1998). In certain embodiments, viral load is reduced to a undetectable level. In certain embodiments, viral load is reduced to a low level of, for example, less than about 20,000 cpm (genome copies per milliliter of serum of the subject), less than about 5000 cpm, less than about 2000 cpm, less than about 500 cpm, or less than about 200 cpm. In certain embodiments, viral load is reduced by at least about 5%, at least about 10%, at least about 25%, at least about 50%, at least about 75%, at least about 90%, at least about 95%, or at least about 99%. In certain embodiments, the methods achieve a sustained viral response, e.g., the viral load is reduced to an undetectable or low level for a period of at least about one month, at least about two months, at least about three months, at least about four months, at least about five months, at least about six months, at least about one year, at least about two years, at least about three years, at least about four years, or at least about five years following cessation of administering a compound of the present disclosure to the subject.

The present disclosure also involves methods of preventing a viral infection in a subject who was or may be exposed to a virus. The methods of preventing a viral infection include administering to the subject who was or may be exposed to a virus an effective amount of an antiviral agent or pharmaceutical composition described herein.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the subject was exposed to a virus. In certain embodiments, the subject may be exposed to a virus. In certain embodiments, the viral infection is prevented by blocking entry of the virus into the cells of the subject.

Another aspect of the present disclosure relates to methods of inhibiting viral replication.

Another aspect of the present disclosure relates to methods of inhibiting viral production.

Another aspect of the present disclosure relates to methods of inhibiting viral activity.

Another aspect of the present disclosure relates to methods of killing a virus.

In certain embodiments, the methods of inhibiting viral replication, viral production, inhibiting viral activity, or killing a virus include contacting a virus with an effective amount of an antiviral agent or pharmaceutical composition described herein.

In certain embodiments, the cell is in vitro. In certain embodiments, the cell is in vivo.

In certain embodiments, the virus is in vitro. In certain embodiments, the virus is in vivo.

In certain embodiments, the effective amount is effective in inhibiting the entry of the virus into a cell of the subject. In certain embodiments, the effective amount is effective in inhibiting an envelope glycoprotein of the virus. In certain embodiments, the effective amount is effective in inhibiting the fusion between the envelope of the virus and the membrane of the cell.

In certain embodiments, the viral infection is Dengue fever. In certain embodiments, the viral infection is Dengue hemorrhagic fever (DHF) or Dengue shock syndrome (DSS). In certain embodiments, the viral infection is yellow fever, West Nile encephalitis. West Nile fever. Japanese encephalitis, or Zika fever, preferably. Zika fever. In certain embodiments, the viral infection is hepatitis B, hepatitis C, fulminant viral hepatitis, severe acute respiratory syndrome (SARS), viral myocarditis, influenza A virus infection, influenza B virus infection, parainfluenza virus infection, measles virus infection, vesicular stomatitis virus infection, rabies virus infection, Ebola virus infection, Junin virus infection, human cytomegalovirus infection, herpes simplex virus 1 infection, poliovirus infection, Marburg virus infection, Lassa fever virus infection, Venezuelan equine encephalitis, Rift Valley fever virus infection, Korean hemorrhagic fever virus infection, Crimean-Congo hemorrhagic fever virus infection, human immunodeficiency virus (HIV) infection, Saint Louise encephalitis, Kyasanur Forest disease, Murray Valley encephalitis, tick-bome encephalitis, Theiler's disease, hepatocellular carcinoma, Kyasanur Forest disease (KFD), Alkhurma disease, Omsk hemorrhagic fever, Rocio encephalitis, wesselsbron disease, Powassan diseas, Israeli turkey meningoencephalitis, Central European tickbome fever, Louping ill, California encephalitis, Border disease, bovine viral diarrhea-mucosal disease, classical swine fever, or bovine hemorrhagic syndrome.

In certain embodiments, the virus is of the Flaviviridae family. In certain embodiments, the virus is of the Flavivirus genus. In certain embodiments, the virus is Dengue virus 2 (DENV2). In certain embodiments, the virus is Dengue virus 1 (DENV1), Dengue virus 3 (DENV3), Dengue virus 4 (DENV4), or Kedougou virus (KEDV). In certain embodiments, the virus is yellow fever virus (YFV), West Nile virus (WNV) Japanese encephalitis virus (JEV), or Zika virus, preferably, Zika virus. In certain embodiments, the virus is a tick-borne virus. In certain embodiments, the virus is Greek goat encephalitis virus (GGEV), Kadam virus (KADV), Krasnodar virus (KRDV). Mogiana tick virus (MGTV) Ngoye virus (NGOV), Sokuluk virus (SOKV), Spanish sheep encephalomyelitis virus (SSEV), Turkish sheep encephalitis virus (TSE), Absettarov virus, Deer tick virus (DT), Gadgets Gully virus (GGYV), Karshi virus, Kyasanur Forest disease virus (KFDV), Alkhurma hemorrhagic fever virus (ALKV), Langat virus (LGTV), Louping ill virus (LIV), Omsk hemorrhagic fever virus (OHFV), Powassan virus (POWV), Royal Farm virus (RFV), Tick-borne encephalitis virus (TBEV), Kama virus (KAMV), Meaban virus (MEAV), Saumarez Reef virus (SREV), or Tyuleniy virus (TYUV). In certain embodiments, the virus is a mosquito-borne virus. In certain embodiments, the virus is Aedes flavivirus, Barkedji virus, Calbertado virus, Cell fusing agent virus, Chaoyang virus, Culex flavivirus, Culex theileri flavivirus, Culiseta flavivirus, Donggang virus, Ilomantsi virus, Kamiti River virus, Lammi virus, Marisma mosquito virus, Nounand virus, Nhumirim virus, Nienokoue virus, Spanish Culex flavivirus, Spanish Ochlerotatus flavivirus, Quang Binh virus, Aroa virus (AROAV), Bussuquara virus (BSQV), Iguape virus (IGUV). Naranjal virus (NJLV), Cacipacore virus (CPCV), Koutango virus (KOUV), Kunjin virus, Ilheus virus (ILHV), Japanese encephalitis virus (JEV), Murray Valley encephalitis virus (MVEV), Alfuy virus, Rocio virus (ROCV), St. Louis encephalitis virus (SLEV), Usutu virus (USUV), West Nile virus (WNV), Yaounde virus (YAOV), Kokobera virus (KOKV), New Mapoon virus (NMV), Stratford virus (STRV). Bagaza virus (BAGV), Baiyangdian virus (BYDV), Duck egg drop syndrome virus (DEDSV), Ilheus virus (ILHV), Israel turkey meningoencephalomyelitis virus (ITV), Jiangsu virus (JSV). Layer flavivirus, Ntaya virus (NTAV), Sitiawan virus (STWV), Tembusu virus (TMUV), Spondweni virus (SPOV), Zika virus (ZIKV), Banzi virus (BANV), Bamaga virus (BGV), Bouboui virus (BOUV), Edge Hill virus (EHV), Jugra virus (JUGV), Saboya virus (SABV), Sepik virus (SEPV), Uganda S virus (UGSV), Wesselsbron virus (WESSV), yellow fever virus (YFV), Batu cave virus, Bukulasa bat virus, Nanay virus, Rabensburg virus (RABV), or Sitiawan virus. In certain embodiments, the virus is Tamana bat virus (TABV), Entebbe bat virus (ENTV), Sokoluk virus, Yokose virus (YOKV). Apoi virus (APOIV), Cowbone Ridge virus (CRV). Jutiapa virus (JUTV), Modoc virus (MODV), Sal Vieja virus (SVV), San Perlita virus (SPV), Bukalasa bat virus (BBV), Carey Island virus (CIV), Dakar bat virus (DBV), Montana myotis leukoencephalitis virus (MMLV), Phnom Penh bat virus (PPBV), or Rio Bravo virus (RBV). In certain embodiments, the virus is Assam virus, Bamaga virus, Cuacua virus, Hanko virus, Mediterranean Ochlerotatus flavivirus, Menghai flavivirus, Nakiwogo virus (NAKV), Ochlerotatus caspius flavivirus, Palm Creek virus, Parramatta River virus, Soybean cyst nematode virus 5, or Xishuangbanna Aedes flavivirus. In certain embodiments, the virus is Aedes flavivirus, Aedes cinereus flavivirus, Aedes vexans flavivirus, or Culex theileri flavivirus. In certain embodiments, the virus is of the Hepacivirus genus, Pegivirus genus, or Pestivirus genus. In certain embodiments, the virus is Hepacivirus A, Hepacivirus B. Hepacivirus C. Hepacivirus D, Hepacivirus E, Hepacivirus F, Hepacivirus G, Hepacivirus H, Hepacivirus I, Hepacivirus J, Hepacivirus K, Hepacivirus L, Hepacivirus M Hepacivirus N Pegivirus A, Pegivirus B, Pegivirus C, Pegivirus D, Pegivirus E, Pegivirus F, Pegivirus G, Pegivirus H, Pegivirus I, Pegivirus J, Pegivirus K, or bovine viral diarrhea virus 1. In certain embodiments, the virus is vesicular stomatitis virus (VSV), vesicular stomatitis virus (VSV) pseudotyped with rabies glycoprotein, vesicular stomatitis virus (VSV) pseudotyped with Ebola glycoprotein. Venezuelan equine encephalitis virus (VEEV), classical swine fever virus, hog cholera virus, papillomavirus, coronavirus, Epstein-Barr virus (EBV), human immunodeficiency virus (HIV), orthomyxovirus, paramyxovirus, arenavirus, bunyavirus, adenovirus, poxvirus, retrovirus, rhabdovirus, picomavirus, or herpesvirus. In certain embodiments, the antiviral agent is a compound described herein.

In certain embodiments, the antiviral agent is a compound of the formula:

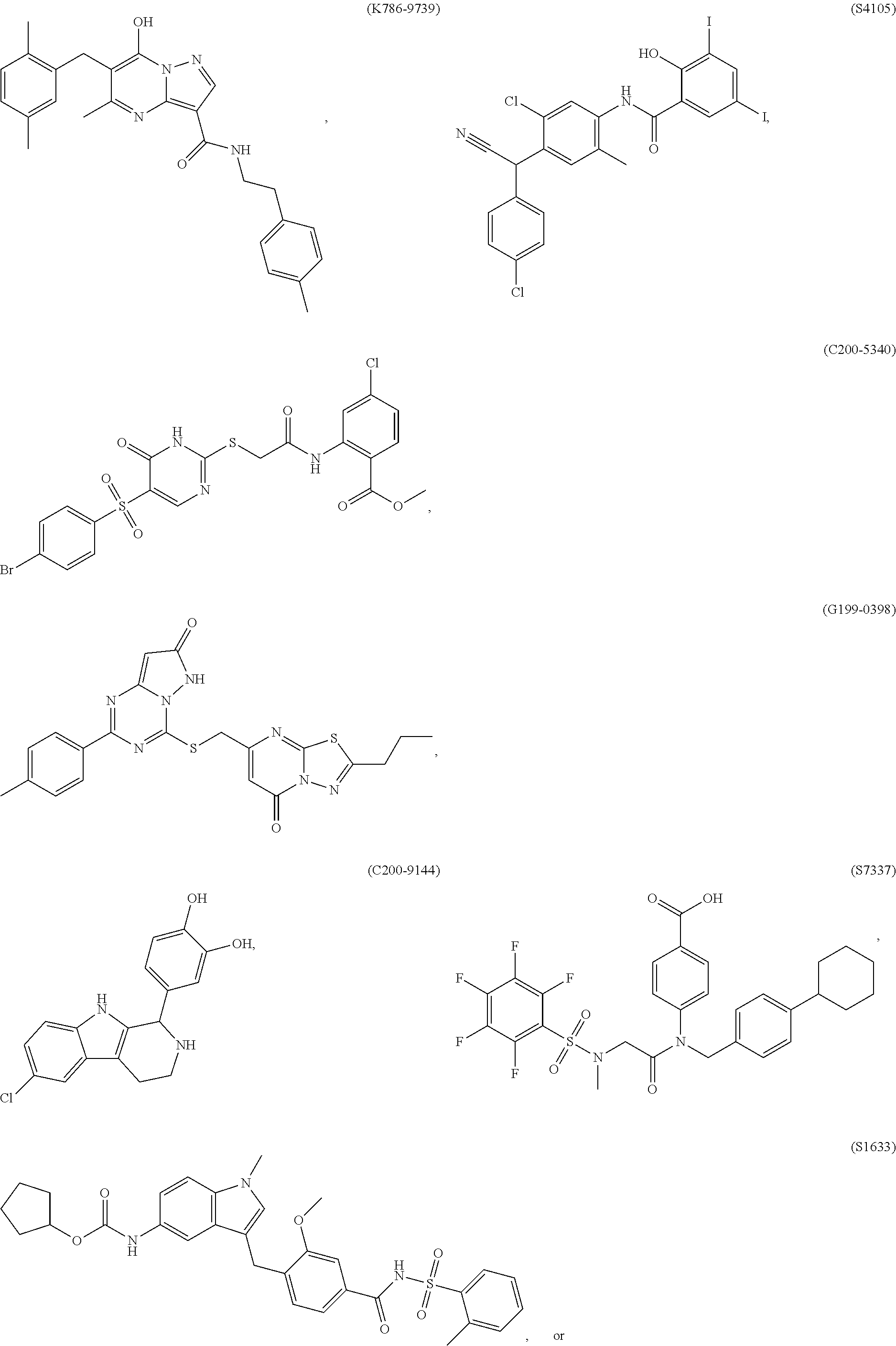

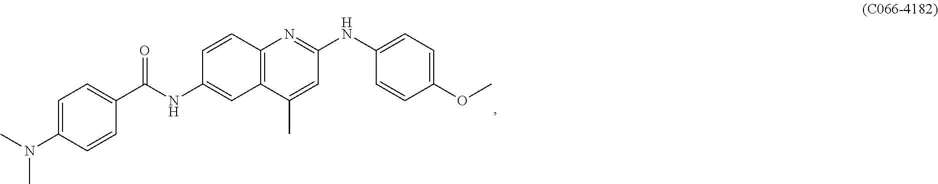

or a pharmaceutically acceptable salt, solvate, hydrate, polymorph, co-crystal, tautomer, stereoisomer, derivative, or prodrug thereof (e.g., a pharmaceutically acceptable salt thereof).

In certain embodiments, the antiviral agent is of the formula

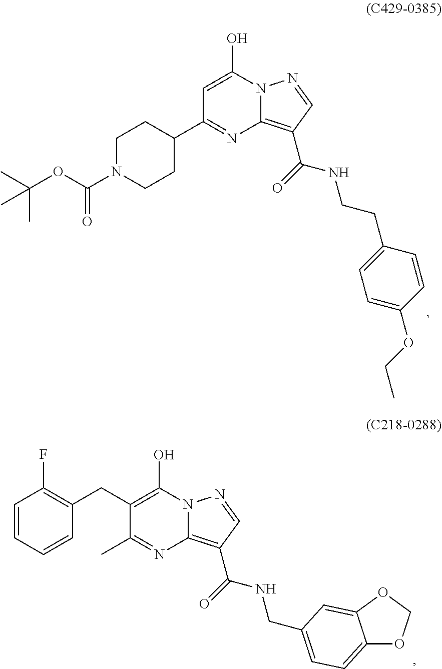 [SMILES: CCOc1ccc(CCNC(=O)c2cnn3c(O)cc(C4CCN(C(=O)OC(C)(C)C)CC4)nc23)cc1 ; Cc1nc2c(C(=O)NCc3ccc4c(c3)OCO4)cnn2c(O)c1Cc1ccccc1F]

or a pharmaceutically acceptable salt, solvate, hydrate, polymorph, co-crystal, tautomer, stereoisomer, derivative, or prodrug thereof.

### EXAMPLES

The examples described herein are offered to illustrate the compounds, pharmaceutical compositions, kits, methods, and/or uses described herein and are not to be construed in any way as limiting their scope.

### Example 1. Preparation and Characterization of Select Compounds

The compounds described herein can be prepared from readily available starting materials using the methods known in the art. Exemplary analytical data for select compounds are shown below.

### Compound JBJ-16-103

¹H NMR (500 MHz, DMSO-d₆) δ 11.68-11.45 (m, 1H), 9.63 (br s, 1H), 8.42-8.28 (m, 1H), 7.26-7.20 (m, 1H), 7.18-7.10 (m, 2H), 7.06 (td, J=7.5, 1.2 Hz, 1H), 4.26 (br s, 1H), 3.86 (s, 2H), 3.48 (br d, J=12.2 Hz, 2H), 3.10 (br s, 2H), 2.78 (br s, 3H), 2.42 (s, 3H), 2.05 (br d, J=12.8 Hz, 2H), 1.80-1.70 (m, 2H). LC/MS (ESI) m/z 398.53 [M+H]⁺.

### Compound JBJ-16-104

¹H NMR (500 MHz, DMSO-d₆) δ 8.64 (br s, 1H), 7.99 (br s, 1H), 7.22-7.16 (m, 1H), 7.15-7.10 (m, 1H), 7.10-7.05 (m, 1H), 7.05-6.99 (m, 1H), 3.86 (s, 2H), 3.25 (t, J=6.1 Hz, 2H), 2.63 (br s, 3H), 2.26 (br s, 3H), 1.93 (br s, 1H), 1.86-1.70 (m, 2H), 1.67-1.52 (m, 2H), 1.36-1.22 (m, 2H), 1.21-1.06 (m, 2H). LC/MS (ESI) m/z 412.57 [M+H]⁺.

### Compound JBJ-16-105

¹H NMR (500 MHz, DMSO-d₆) δ 11.59 (br s, 1H), 9.01 (t, J=6.0 Hz, 1H), 8.54-8.51 (m, 1H), 8.43 (s, 1H), 7.78 (td, J=7.7, 7.1 Hz, 1H), 7.37 (d, J=7.9 Hz, 1H), 7.31-7.26 (m, 1H), 7.26-7.20 (m, 1H), 7.18-7.10 (m, 1H), 7.08-7.03 (m, 1H), 4.57 (d, J=5.8 Hz, 2H), 3.86 (s, 2H), 2.41 (s, 3H). LC/MS (ESI) m, 392.45 [M+H]⁺.

### Compound ZNL-01-019

¹H NMR (500 MHz, DMSO-d₆) δ 11.55 (s, 1H), 8.42 (t, J=5.6 Hz, 1H), 8.30 (s, 1H), 7.37-7.33 (d, J=8.7 Hz, 2H), 7.26 (d, J=8.4 Hz, 2H), 6.52 (s, 1H), 5.75 (s, 1H), 3.47 (dd, J=13.2, 6.8 Hz, 2H), 3.13 (s, 2H), 2.84 (t, J=7.2 Hz, 2H), 2.78 (d, J=4.5 Hz, 6H), 2.68 (s, 3H), 2.46 (s, 3H). MS m/z 431.29 [M+H]⁺.

### Compound ZNL-01-039

MS m/397.35 [M+H]⁺.

### Compound ZNL-01-040

MS m/z 395.32 [M+H]⁺.

### Compound ZNL-02-081

¹H NMR (500 MHz, DMSO-d₆) δ 11.83 (s, 1H), 9.95 (s, 1H), 8.52 (s, 1H), 7.64 (d, J=9.0 Hz, 2H), 7.02-6.86 (m, 2H), 5.46 (d, J=1.9 Hz, 1H), 4.02 (q, J=7.0 Hz, 2H), 2.46-2.37 (m, 1H), 1.33 (t, J=7.0 Hz, 3H), 1.16-1.07 (m, 2H), 1.00-0.92 (m, 2H). MS m/z 393.32 [M+H]⁺.

### Compound ZNL-02-082

¹H NMR (500 MHz, DMSO-d₆) δ 11.68 (s, 1H), 10.00 (s, 1H), 8.68 (s, 1H), 8.57 (s, 1H), 8.43 (s, 1H), 7.67-7.59 (m, 2H), 6.93 (d, J=9.0 Hz, 2H), 5.77 (s, 1H), 4.01 (q, J=7.0 Hz, 2H), 3.44 (d, J=12.2 Hz, 1H), 3.22 (t, J=11.9 Hz, 1H), 2.94 (d, J=11.0 Hz, 2H), 2.10 (t, J=10.5 Hz, 2H), 1.77 (dd, J=23.4, 11.9 Hz, 2H), 1.33 (t, J=7.0 Hz, 3H). MS m/z 382.26 [M+H]⁺.

### Compound ZNL-02-086

¹H NMR (500 MHz, DMSO-d₆) δ 11.52 (s, 1H), 10.06 (s, 1H), 8.58 (s, 1H), 7.91 (s, 2H), 7.64 (d, J=9.0 Hz, 2H), 7.44 (t, J=8.8 Hz, 2H), 6.98-6.87 (m, 2H), 6.28 (s, 1H), 4.02 (q, J=6.9 Hz, 2H), 1.33 (t, J=7.0 Hz, 3H). MS m/z 393.31 [M+H]⁺.

### Compound ZNL-02-087

¹H NMR (500 MHz, DMSO-d₆) δ 11.66 (s, 1H), 9.96 (s, 1H), 8.55 (s, 1H), 7.83-7.49 (m, 2H), 7.02-6.86 (m, 2H), 5.80 (d, J=1.8 Hz, 1H), 4.55 (d, J=13.2 Hz, 1H), 4.02 (q, J=7.0 Hz, 2H), 3.95 (d, J=13.6 Hz, 2H), 3.27-3.12 (m, 1H), 3.07 (t, J=11.9 Hz, 1H), 2.03 (s, 3H), 1.92 (t, J=13.2 Hz, 2H), 1.62 (qd, J=12.5, 4.2 Hz, 1H), 1.45 (qd J=12.6, 4.3 Hz, 1H), 1.33 (t, J=7.0 Hz, 3H). MS m, 424.24 [M+H].

### Compound ZNL-02-088

¹H NMR (500 MHz, DMSO-d₆) δ 11.71 (s, 1H), 9.96 (s, 1H), 8.55 (s, 1H), 7.64 (d, J=9.0 Hz, 2H), 6.93 (d, J=9.0 Hz, 2H), 5.86 (d, J=1.6 Hz, 1H), 4.46 (d, J=13.3 Hz, 1H), 4.07-3.95 (m, 3H), 3.32-3.23 (m, 2H), 2.92 (t, J=12.3 Hz, 1H), 2.05 (dd, J=24.7, 11.1 Hz, 2H), 1.85-1.57 (m, 2H), 1.33 (t, J=7.0 Hz, 3H). MS m/z 478.23 [M+H]⁺.

### Example 2. Compound Screening

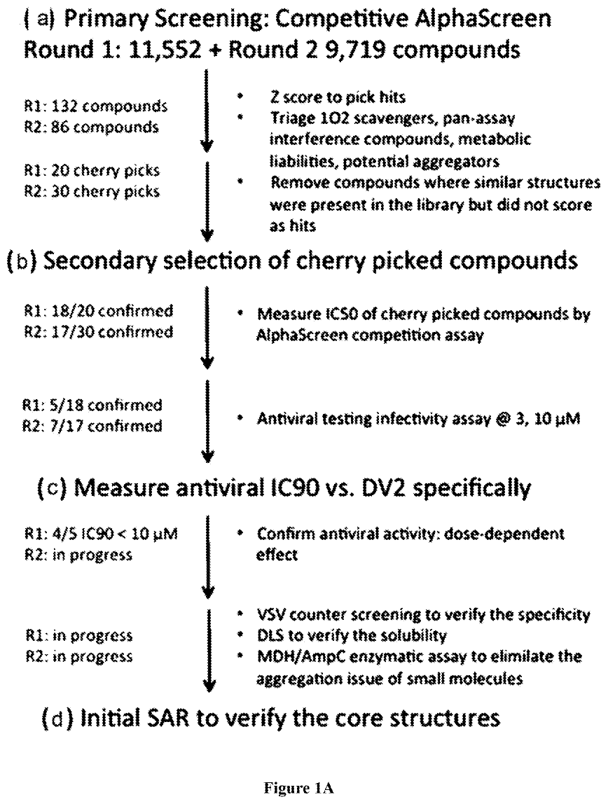

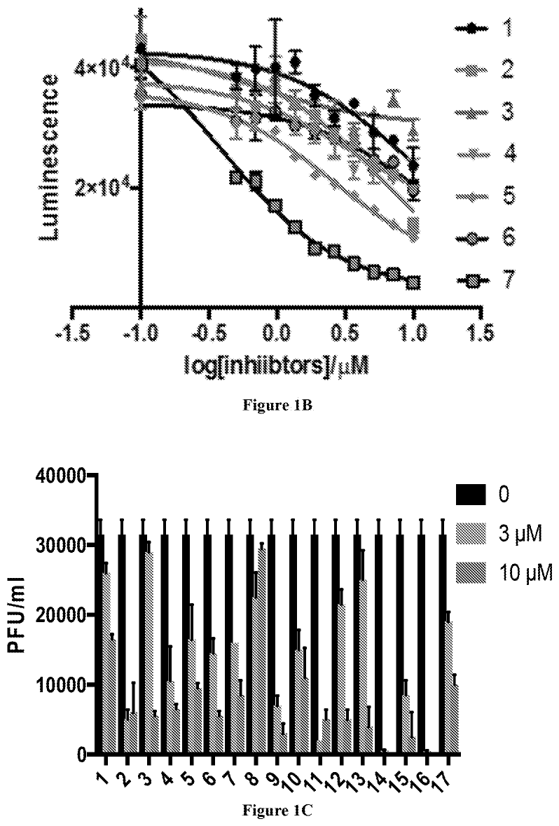

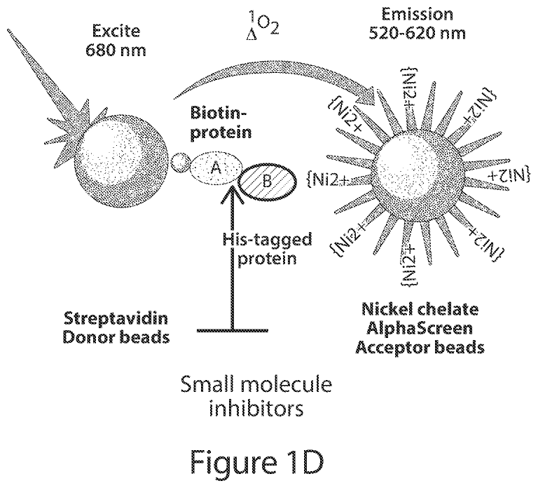

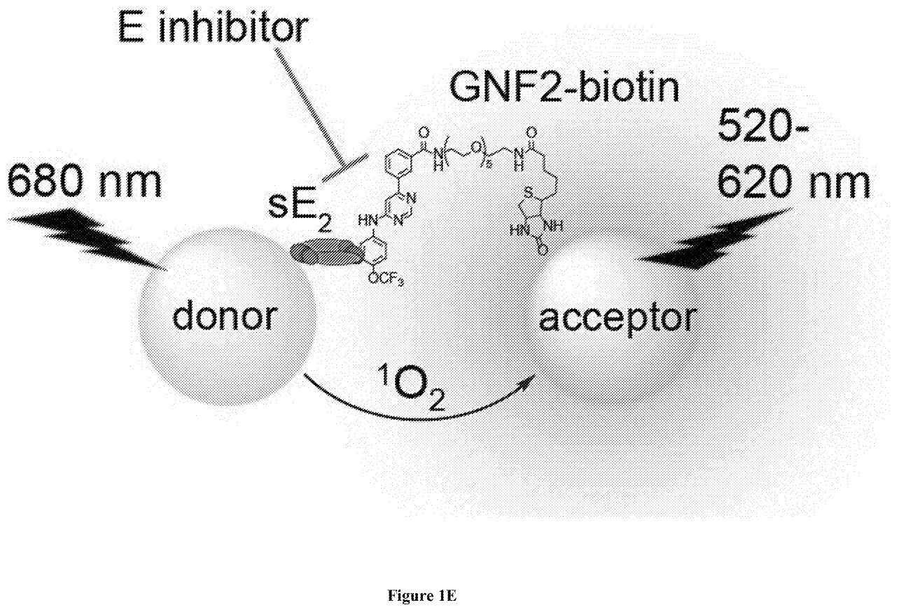

Known DENV inhibitors may be limited in their potential to be developed as preclinical candidates if we are unable to develop a derivative with sufficiently potent pan-serotype activity and/or because some of the known DENV inhibitors still exhibit cytotoxicity at concentrations of about 10- to 20-fold above those required for their antiviral activity due to off-target effects. To address this potential problem, we have used known DENV inhibitors to develop an AlphaScreen assay designed to identify compounds that compete for binding on envelop protein (FIG. 1D). Also see FIG. 1E.

The AlphaScreen is a bead-based proximity assay that permits measurement of biomolecular interactions of pico- to milli-molar affinities in microplate format. Following excitation of donor beads, energy is transferred to acceptor beads if analytes conjugated to donor and acceptor beads interact. To establish an AlphaScreen assay for inhibitors targeting the envelope glycoprotein (e.g., the dimer of the envelope glycoprotein (E2)) of DENV, we synthesized biotinylated derivatives of GNF2 and demonstrated that the conjugates (biotinylated derivatives of GNF2) still bind the soluble E2 (sE2) with low micromolar affinity in bio-layer interferometry assays and retain the anti-DENV activity of the parent compound (GNF2)³¹. Mixing GNF2-biotin (FIG. 3) immobilized on streptavidin donor beads with His6-tagged DENV2 sE2 on acceptor beads produced an AlphaScreen signal that can be competed away in a dose-dependent fashion by free GNF2 and compound 3-110-22³⁰ (Table 1) but not by the negative control. We performed cross-titration experiments to optimize the molar concentrations of the probe (GNF2-biotin) and envelope glycoprotein for the assay. We also tested different orders of the addition of the assay components to achieve the best signal-to-noise ratio. In the initial experiments in 384-well plates using compound 3-110-22 as a positive control and DMSO as a negative control, the assay exhibited a signal-to-noise ratio (S/B) of 11 and a Z′ value of 0.53.

In the primary screening, about 20,000 compounds at the concentration of 10 μM (Selleck Known Bioactive library) or 5 μg/ml (ChemDiv library) in duplicate were applied in the AlphaScreen based competition assay using automation equipment in ICCB-L. Those automated assays performed a S/B about 3.5 and Z′ value from 0.45 to 0.65. We initially picked out 218 compounds (1.02% of the total library) by Z value (−2.5 to −3: medium; <−3: strong) and filtered based on triage 102 scavengers, pan-assay interference compounds, metabolic liabilities, and potential aggregators. The compounds where similar structures were present in the library but did not score as hits were removed. Of 218 compounds filtered, 50 were cherry picked to verify the dose-dependent competition activity.

The confirmed hit compounds were purchased. IC₅₀ values for inhibition of the DENV2 sE2 interaction with GNF2-biotin in the AlphaScreen assay were measured. Thirty-five out of fifty compounds showed efficient competition activity of GNF2-biotin to DENV2 sE2 (IC₅₀<10 μM). In addition, initial antiviral activity assay was performed. Two concentrations of inhibitors (3 or 10 μM) were chosen to verify whether the inhibitors could reduce single-cycle viral yield when treatment with the inhibitors was limited to an initial 45 min preincubation with viral inoculum and an initial one-hour infection period but is otherwise absent in the rest of time. Twelve out of thirty-five compounds (at 10 μM or less) showed more than 90% inhibition of viral yield. (FIGS. 1A to 1C).

### Example 3. Confirmation and Characterization of Select Screening Hits

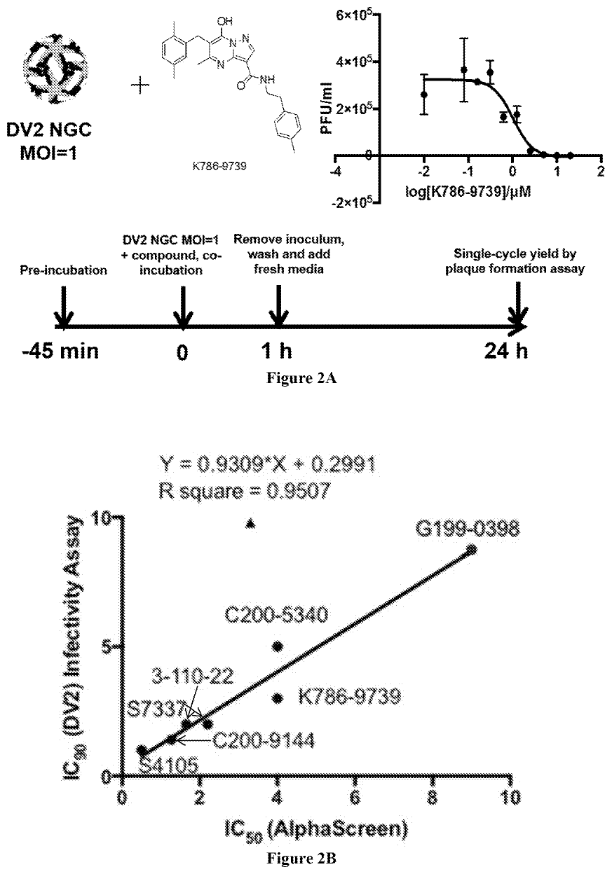

IC₉₀ values were determined for select inhibitors of DENV envelope glycoprotein to show the inhibitors' antiviral potency. We have demonstrated that the IC₉₀ values are well-correlated with the IC₅₀ values (which show the competition activity) in the AlphaScreen assay. (FIGS. 24 and 2B). In addition, we have recently developed a label-free, bio-layer interferometry assay on a CMI-Longwood ForteBio OctetRED384 system that enables us to measure equilibrium affinity constants (K_{D}) as well as kinetic on and off rates (kₒₙ and k_{off}) for the interaction of our inhibitors with recombinant sE2 in 384-well format. Using this assay with our screening inhibitors, we have demonstrated that the IC₅₀ values in the AlphaScreen assay are also correlated with the binding affinity. We screened out 8 compounds, of which compounds S4105, K786-9739, S7337, and C200-9144 showed potent inhibition versus DENV2 (IC₉₀ 3 μM) (Table 1). Some compounds (e.g., compounds S4105, K786-9739, and C200-9144 showed herein) exhibited cytotoxicity (as shown by CC₅₀ values) at concentrations more than 30-fold of their effective antiviral concentrations (Table 1). Other compounds showed stability (e.g., compound G199-0398, whose Ti is about 70 min) and/or specificity (e.g., compound C200-5340) (able 1).

### Example 4. Antiviral Activity (Viral Infectivity) Assays

For the viral infectivity assay, virus inocula were diluted in EBSS to achieve a multiplicity of infection (MOI) of 1, and were pre-incubated with the given small molecule at varying concentrations for 45 min at 37° C. The mixture was then added to cells for 1 hour at 37° C. to allow infection, after which the inoculum was removed and the cells were washed with 1×PBS to remove unbound virus and compound. Cells were overlaid with medium lacking inhibitor and incubated at 37° C. for 20-24 hours, corresponding to a single cycle of infection. Culture supernatants were harvested at this time, and the yield of infectious particles produced was quantified by plaque-forming assay. For initial antiviral screening of HTS “hits,” compounds were tested for activity at 3 and 10 μM. For IC₉₀ value determination, viral yield (plaque-forming units per milliliter) was plotted versus the log of the inhibitor concentration, and non-linear regression analysis of the data (Graphpad Prism) was performed to determine the concentration at which viral yield is reduced 10-fold.

### Example 5. Bio-Layer Interferometry (BLI) Assays, for Determination of Dissociation Constant (K_{D}) Values

K_{D} measurements were performed on an Octet RED384 system (ForteBio). Recombinant, soluble, biotinylated DENV2 sE₂ protein was immobilized on super-streptavidin (SSA) biosensor tips, after which the tips were quenched with biocytin and then equilibrated in buffer prior to baseline collection and then data acquisition in the presence of varying compound concentrations. BLI mixtures (80 μL) were prepared in wells of a 384-well black tilted-bottom plate (ForteBio), and the measures were monitored by Octet RED384 system (ForteBio). 1.6 μg of the biotinylated protein was loaded on an SSA biosensor tip (ForteBio) for 600 seconds and then quenched with 0.8 μg biocytin for 120 seconds. The SSA biosensors were then equilibrated in reaction buffer [1× Kinetic buffer (ForteBio), 1×PBS, 2% DMSO] for 180 seconds prior to baseline collection. Association with small molecules was monitored for 120 seconds with inhibitor concentrations that ranged from 50 nM to 20 μM dissociation was performed in reaction buffer and monitored for 120 seconds. Equilibrium dissociation constants (K_{D}) values were determined by plotting the local fit maximum response (nm) as a function of small molecule concentrations (μM) using ForteBio software and GraphPad Prism. Titration curves were fit to the following steady-state analysis equation: “Response=(Rₘₐₓ*Conc)/K_{D}+Conc”, where Rₘₐₓ is the local fit response maximum; “Conc” is the concentration of small molecule; and K_{D} is the equilibrium dissociation constant. The data showed that the antiviral activity was well-correlated with K_{d} for binding to recombinant, prefusion E.

### Example 6. VSV-eGFP Counter Screen

Virus inocula were diluted in EBSS to achieve a multiplicity of infection (MOI) of 1, and were pre-incubated with the given small molecule at varying concentrations for 45 min at 37° C. 100 nM bafilomycin was used as a positive control inhibitor of VSV-eGFP entry. The virus-inhibitor mixture was then added to cells for 1 hour at 37° C. to allow infection, after which the inoculum was removed, and the cells were washed with 1×PBS to remove unbound virus and compound. Cells were overlaid with medium lacking inhibitor and incubated at 37° C. for 6 hours, corresponding to a single cycle of infection. Following removal of the supernatants, the cells were washed with 1×PBS and overlayed with PBS and then imaged. Fluorescence (excitation 488 nm, emission 525 nm) was measured using a Typhoon FLA 9500 (GE Healthcare Life Sciences) and quantified using ImageQuant TL (GE Healthcare Life Sciences).

### Example 7. Non-Specific Enzyme Inhibition Assays

AmpC Beta-Lactamase Assay.

The AmpC β-lactamase was a kind gift from the Shoichet lab (UCSF). The inhibitor was serially diluted (two-fold dilution series from 100 μM) and pre-incubated with 10 nM enzyme in working buffer (50 mM potassium phosphate, pH 7.0) at room temperature for 5 min. Nitrocefin (100 μM, VWR) was added to the solution and carefully mixed. Absorbance of the final mixture was immediately monitored at 470 nm for 3 min.

Malate Dehydrogenase (MDH) Assay.

Small molecule inhibitors were serially diluted (2-fold dilution series from 100 μM) and were mixed with 200 μM oxaloacetic acid (VWR) and 200 μM NADH (VWR) in working buffer (100 mM potassium phosphate, pH 7.0). Malate dehydrogenase (EMD Millipore) was added to a final concentration of 17.5 nM, and absorbance was immediately monitored at 340 nm for 5 minutes.

For both AmpC and MDH assays, the final concentration of DMSO was 2% for all samples. All assays were repeated in the presence of 0.01% Triton X-100. IC₅₀ values of compound 3-110-22 presented in Table 1 were representative data from two independent experiments; values for the other compounds were measured once for each enzymatic assay.

### Example 8. Dynamic Light Scattering Assays

Different concentrations of small molecule solutions were prepared in 110 μL of 1×PBS buffer with 2% DMSO (vol/vol). Solutions were centrifuged at 21130 g for 10 minutes (room temperature). No precipitation by naked eyes was observed. Supernatant (100 μl solution) was transferred to a low-volume quartz batch cuvette (ZEN2112, Malvern). Particle size was measured on a Zetasizer Nano instrument (Malvern). The values presented in Table 1 were averages of more than 11 technical replicates.

### Example 9. Cytotoxicity Assays

BHK21 cells (MEM with 2% FBS) were incubated with varying concentrations of inhibitor in a 96-well white plate for 24 hours at 37° C. and 5% CO₂. CellTiter-Glo (Promega) solution was used to measure viability following the manufacturer's instructions. Luminescence was measured using a Biotek Synergy plate reader. Data were plotted versus the log₁₀ inhibitor concentration, and non-linear regression analysis (Graphpad Prism) was used to determine CC₅₀ values, defined as the inhibitor concentration required to cause 50% loss of cell viability. The maximum concentration tested was 100 μM. Values presented in Table 1 are the average of two or more independent experiments.

### Example 10. Plaque Reduction Assays

DENV1, 2, 3, 4 were tested on BHK-21 cells, and ZIKV was tested on Vero cells. Virus inocula were diluted in EBSS to 2500 pfu/ml as the final concentration, and were pre-incubated with different concentrations of small molecule inhibitors (2% DMSO vol/vol final concentration) for 45 min at 37° C., 5% CO₂. The mixture (200 μl, 500 pfu of virus) was then added to cells for 1 hour (37° C., 5% CO₂) to allow infection, after which the inoculum was removed, and the cells were washed with 1×PBS to remove unbound virus and compound. Cells were overlaid with carboxylmethylcellulose and incubated at 37° C., 5% CO₂ to allow the formation of plaques (4 days for DENV1, 2, 3, 4 and 2-3 days for ZIKV). The cells were fixed and plaques visualized by staining of the cell monolayers with crystal violet Empirical analysis was performed to determine the PRNT₅₀ (EC₅₀ (PRNT)) value, defined as the inhibitor concentration needed to reduce plaque formation by 50%.

TABLE 1
Exemplary characterization of select compounds.

| | | | IC₉₀ | | | | |
|---|---|---|---|---|---|---|---|
| | IC₅₀ | | (DENV2 | | | CC₅₀ | |
| Compound | (AlphaScreen) | K_{d} | infectivity) | VSV | VSV | (cytotoxicity) | |
| Number | (μM) | (μM) | (μM) | 5 μM | 10 μM | (μM) | SI_{50/90} |
| 3-110-22 | 1.1 ± 0.7 | 0.5 | 2.8 ± 1.3 | 41 | 43 | 69 | 25 |
| | (n = 4) | | (n = 7) | | | | |
| S4105 | 0.6 ± 0.2 | 0.9 | 0.8 ± 0.3 | 60 | 64 | 50 | >50 |
| | (n = 4) | | (n = 2) | | | | |
| K786-9739 | 3.0 ± 1.7 | 1.3 | 3.1 ± 0.1 | 35 | 63 | 81 | 26 |
| | (n = 4) | | (n = 2) | | | | |
| C200-5340 | 3.0 ± 1.1 | 7.1 | 7.2 ± 3.5 | 20 | 51 | >>50 | >10 |
| | (n = 4) | | (n = 2) | | | | |
| G199-0398 | 7.0 ± 3.0 | ND | 8.8 ± 0.2 | 33 | 59 | 68 | 8 |
| | (n = 4) | | (n = 2) | | | | |
| C200-9144 | 2.6 ± 1.3 | 1.2 | 1.2 ± 0.4 | 76 | 77 | >50 | >40 |
| | (n = 3) | | (n = 2) | | | | |
| S7337 | 4.1 ± 1.8 | 1.3 | 2.7 ± 1.0 | 17 | 66 | 12.7 | 5 |
| | (n = 3) | | (n = 2) | | | | |
| S1633 | 3.1 ± 1.0 | 5.2 | 7.0 ± 2.8 | 12 | 40 | 50 | 7 |
| | (n = 3) | | (n = 2) | | | | |
| C066-4182 | 1.4 ± 1.1 | 2.2 | 17 ± 4.5 | 5 | 22 | >>50 | >2.5 |
| | (n = 3) | | (n = 2) | | | | |
| | | | IC₅₀ | | IC₅₀ | Particle | Particle size |
| | T_{1/2} (mouse | IC₅₀ | (MDH, | IC₅₀ | (AmpC, | size (no | (with Tween |
| Compound | microsome) | (MDH) | Triton) | (AmpC) | Triton) | Tween ®) | 20 ®) |
| Number | (minutes) | (μM) | (μM) | (μM) | (μM) | (nm) | (nm) |
| 3-110-22 | 13.1 | 44.6 | >100 | >100 | >100 | 1069 (20 μM) | 7.5 (20 μM) |
| S4105 | >120 | 0.3 | >100 | 19.5 | >100 | 82 (40 μM) | 13.1 (40 μM) |
| K786-9739 | 14.9 | 20.1 | >100 | 36.2 | >100 | 628 (40 μM) | 7.1 (40 μM) |
| C200-5340 | 4.2 | 42.4 | >100 | 66 | >100 | 966 (40 μM) | 7.5 (40 μM) |
| G199-0398 | 70.6 | >100 | >100 | >100 | >100 | 268 (40 μM) | 7.2 (40 μM) |
| C200-9144 | 55.6 | 82.1 | >100 | 73.5 | >100 | 1443 (40 μM) | 8.3 (40 μM) |
| S7337 | 3.2 | 86.4 | >100 | >100 | >100 | 144 (40 μM) | 8.2 (40 μM) |
| S1633 | 37.7 | 48.2 | >100 | 36.6 | >100 | 823 (40 μM) | 9.2 (40 μM) |
| C066-4182 | 5.3 | 36.5 | >100 | 24.3 | >100 | ND | 63, 528 |
| | | | | | | | (40 μM) |

In Table 1, “ND” denotes “not detected, “VSV” denotes the percentage inhibition of single-cycle VSV-eGFP infection with an antiviral agent, T_{1/2}” indicates the mouse microsomal stability of an antiviral agent “IC₅₀ (MDH)” and “IC₅₀ (AmpC)” denote the concentration of an antiviral agent that inhibits 50% activity of the enzymes without detergent, “IC₅₀ (MDH, Triton)” and “IC₅₀ (AmpC, Triton)” denote the concentration of an antiviral agent that inhibits 50% activity of the enzymes with detergent, and the particle size was measured by DLS with or without detergent.

During preclinical drug discovery, it remains a challenge to enable early elimination of candidate molecules that may have non-specific, off-target activities. Colloidal aggregation of organic molecules is a major mechanism for artefactual inhibition of targets. It is now well accepted that promiscuous inhibition caused by small molecule aggregation is a major source of false positive results in high-throughput screening³². We eliminated colloidal aggregators, using two criteria^{33,34}: detergent-dependent inhibition of an established counter-screening enzyme, AmpC β-lactamase (AmpQ) and malate dehydrogenase, and observation of colloidal particles by dynamic light scattering (DLS). To be considered an aggregator, a molecule had to inhibit AmpC β-lactamase or malate dehydrogenase with an IC₅₀ value lower than 100 μM, have that inhibition much diminished or eliminated by addition of 0.01% Triton X-100 (Triton) and form particles characteristic of aggregators observable by dynamic light scattering (DLS). We also tested the inhibitory activity of those compounds against vesicular stomatitis virus (VSV)-enhanced green fluorescent protein (eGFP) as another counter screen (VSV counter screen) to verify the specificity of the screening hits. Compounds C200-5340, C200-9144, and S7337 emerged less aggregation issue based on both enzymatic inhibition assay and less potency in VSV counter screen. Compound G199-0398 showed no activity vs. AmpC and MDH enzymes but showed strong inhibition efficiency against VSV-eGFP (Table 1).

Shown in Table 2 are exemplary AlphaScreen IC₅₀ and antiviral activity data for select compounds.

TABLE 2
Exemplary AlphaScreen IC₅₀ and antiviral activity data for
select compounds.

| | IC₅₀ | | |
|---|---|---|---|
| Compound | (AlphaScreen) | (Anti-DENV2)% | (Anti-DENV2)% |
| Number | (μm) | at 3 μM | at 10 μM |
| JBJ-16-103 | No competition | | |
| JBJ-16-104 | No competition | | |
| JBJ-16-105 | >10 | 20 | 86 |

TABLE 3
Exemplary data for compounds C429-0385 and C218-0288.

| | IC₅₀ | EC₅₀ | CC₅₀ | IC₉₀ (DENV2 |
|---|---|---|---|---|
| Compound | (AlphaScreen) | (PRNT) | (toxicity) | infectivity) |
| # | (μm) | (μm) | (μm) | (μm) |
| C429-0385 | 7.6 | 0.6 | >100 | |
| | 5.3 | | | About 3 |
| C218-0288 | 1.9 | | | |

In Tables 1 to 3, The term “IC₅₀” refers to the concentration of an antiviral agent that inhibits 50% luminescence signal in the AlphaScreen competition assay.

### REFERENCES

(1) Bhatt, S.; Gething, P. W.; Brady, O. J.; Messina, J. P.; Farlow, A. W.; Moyes, C. L.; Drake, J. M.; Brownstein, J. S.; Hoen, A. G.; Sankoh, O.; Myers, M. F.; George, D. B.; Jaenisch, T.; Wint, G. R. W.; Simmons, C. P.; Scott, T. W.; Farrar, J. J.; Hay, S. I. The Global Distribution and Burden of Dengue. Nature 2013, 496 (7446), 504-507.
(2) Sabchareon, A.; Wallace. D.; Sirivichayakul, C.; Limkittikul, K.; Chanthavanich, P.; Suvannadabba, S.; Jiwariyavej, V.; Dulyachai, W.; Pengsaa, K.; Wartel, T. A.; Moureau, A.; Saville, M.; Bouckenooghe, A.; Viviani, S.; Tornieporth, N. G.; Lang, J. Protective Efficacy of the Recombinant, Live-Attenuated, CYD Tetravalent Dengue Vaccine in Thai Schoolchildren; A Randomised, Controlled Phase 2b Trial. The Lancet 2012, 380 (9853), 1559-1567.
(3) Safety and Immunogenicity of a Recombinant Tetravalent Dengu . . . : The Pediatric Infectious Disease Journal http://journals.lww.com/pidj/Fulltext/2013/10000/Safety_and_Immunogenicity_of_a_Recombinant.18.aspx (accessed Apr. 24, 2017).
(4) Capeding, M. R., Tran, N. H., Hadinegoro, S. R. S.; Ismail, H. I. H. M.; Chotpitayasunondh, T.; Chua, M. N.; Luong, C. Q.; Rusmil, K.; Wirawan, D. N.; Nallusamy, R.; Pitisuttithum, P.; Thisyakom, U.; Yoon, I.-K; van der Vliet, D.; Langevin, E.; Laot, T.; Hutagalung, Y.; Frago, C.; Boaz, M.; Wartel, T. A.; Tornieporth, N. G.; Saville, M.; Bouckenooghe, A. Clinical Efficacy and Safety of a Novel Tetravalent Dengue Vaccine in Healthy Children in Asia: A Phase 3, Randomised, Observer-Masked, Placebo-Controlled Trial. The Lancet 2014, 384 (9951), 1358-1365.
(5) Capeding, M. R.; Laot, T. M.; Boaz, M.; Wartel, T. A.; Crevat, D. Imunogenicity and Safety of a Tetravalent Dengue Vaccine during a Five-Year Follow-up Period. Trials Vaccinol. 2015, 4, 19-23.
(6) Hadinegoro, S. R.; Arredondo-Garcia. J. L.; Capeding, M. R.; Deseda, C.; Chotpitayasunondh, T.; Dietze, R.; Hj Muhammad Ismail, H. I.; Reynales, H.; Limkittikul, K.; Rivera-Medina, D. M.; Tran, H. N.; Bouckenooghe, A.; Chansinghakul, D.; Cortds, M.; Fanouillere, K.; Forrat, R.; Frago, C.; Gailhardou, S.; Jackson, N.; Noriega, F.; Plennevaux, E.; Wartel, T. A.; Zambrano, B.; Saville, M. Efficacy and Long-Term Safety of a Dengue Vaccine in Regions of Endemic Disease. N. Engl. J. Med. 2015, 373 (13), 1195-1206.
(7) Stephenson, J. R. Understanding Dengue Pathogenesis: Implications for Vaccine Design. Bull. World Health Organ. 2005, 83 (4), 308-314.
(8) Edelman, R.; Hombach, J. “Guidelines for the Clinical Evaluation of Dengue Vaccines in Endemic Areas”: Summary of a World Health Organization Technical Consultation. Vaccine 2008, 26 (33), 4113-4119.
(9) Murphy, B. R.; Whitehead, S. S. Immune Response to Dengue Virus and Prospects for a Vaccine. Annu. Rev. Immunol. 2011, 29 (1), 587-619.
(10) Barba-Spaeth, G.; Dejnirattisai, W.; Rouvinski, A.; Vaney, M.-C.; Medits, I.; Sharma, A.; Simon-Lorière, E.; Sakuntabhai, A.; Cao-Lormeau, V.-M.; Haouz, A.; England, P.; Stiasny, K.; Mongkolsapaya, J.; Heinz, F. X.; Screaton, G. R.; Rey, F. A. Structural Basis of Potent Zika-Dengue Virus Antibody Cross-Neutralization. Nature 2016, 536 (7614),48-53.
(11) Dejnirattisai, W.; Supasa, P.; Wongwiwat, W.; Rouvinski, A.; Barba-Spaeth, G.; Duangchinda. T.; Sakuntabhai, A.; Cao-Lormeau, V.-M.; Malasit, P.; Rey, F. A.; Mongkolsapaya, J.; Screaton, G. R. Dengue Virus Sero-Cross-Reactivity Drives Antibody-Dependent Enhancement of Infection with Zika Virus. Nat. Immunol. 2016, 17 (9),1102-1108.
(12) Priyamvada, L.; Quicke, K. M.; Hudson, W. H.; Onlamoon, N.; Sewatanon, J.; Edupuganti, S.; Pattanapanyasat, K.; Chokephaibulkit, K.; Mulligan, M. J.; Wilson, P. C.; Ahmed, R.; Suthar, M. S.; Wrammert, J. Human Antibody Responses after Dengue Virus Infection Are Highly Cross-Reactive to Zika Virus. Proc. Nat. Acad. Sci. 2016, 113 (28),7852-7857.
(13) Stettler, K.; Beltramello, M.; Espinosa, D. A.; Graham, V.; Cassotta, A.; Bianchi, S.; Vanzetta, F.; Minola, A.; Jaconi, S.; Mele, F.; Foglierini, M.; Pedotti, M.; Simonelli, L.; Dowall, S.; Atkinson, B.; Percivalle, E.; Simmons, C. P.; Varani, L.; Blum, J.; Baldanti, F.; Cameroni, E.; Hewson, R.; Harris, E.; Lanzavecchia, A.; Sallusto, F.; Corti, D. Specificity, Cross-Reactivity and Function of Antibodies Elicited by Zika Virus Infection. Science 2016, aaf8505.
(14) Swanstrom, J. A.; Plante, J. A.; Plante, K. S.; Young, E. F.; McGowan, E.; Gallichotte, E. N.; Widman, D. G.; Heise, M. T.; Silva. A. M. de; Baric, R. S. Dengue Virus Envelope Dimer Epitope Monoclonal Antibodies Isolated from Dengue Patients Are Protective against Zika Virus. mBio 2016, 7 (4), e01123-16.
(15) Bruno Canard. Antiviral Research and Development Against Dengue Virus; 2012.
(16) Lim, S. P.; Wang, Q.-Y.; Noble, C. G.; Chen, Y.-L.; Dong, H.; Zou, B.; Yokokawa, F.; Nilar, S.; Smith, P.; Beer, D.; Lescar, J.; Shi, P.-Y. Ten Years of Dengue Drug Discovery: Progress and Prospects. Antiviral Res. 2013, 100 (2), 500-519.
(17) Kuhn, R. J.; Zhang, W.; Rossmann, M. G.; Pletnev, S. V.; Corver, J.; Lenches. E.; Jones, C. T.; Mukhopadhyay, S.; Chipman, P. R.; Strauss, E. G.; Baker, T. S.; Strauss, J. H. Structure of Dengue Virus: Implications for Flavivirus Organization, Maturation, and Fusion. Cell 2002, 108 (5), 717-725.
(18) Modis, Y.; Ogata. S.; Clements, D.; Harrison, S. C. A Ligand-Binding Pocket in the Dengue Virus Envelope Glycoprotein. Proc. Natl. Acad. Si. 2003, 100 (12), 6986-6991.
(19) Zhang, X.; Ge. P.; Yu. X.; Brannan, J. M.; Bi, G.; Zhang. Q.; Schein, S.; Zhou, Z. H. Cryo-EM Structure of the Mature Dengue Virus at 3.5-Å Resolution. Nat. Struct. Mol. Biol. 2013, 20 (1), 105-110.
(20) Krishnan, M. N.; Sukumaran, B.; Pal, U.; Agaisse, H.; Murray, J. L.; Hodge, T. W.; Fikrig, E. Rab 5 Is Required for the Cellular Entry of Dengue and West Nile Viruses. J. Virol. 2007, 81 (9), 4881-4885.
(21) Acosta, E. G.; Castilla. V.; Damonte, E. B. Functional Entry of Dengue Virus into Aedes albopictus Mosquito Cells Is Dependent on Clathrin-Mediated Endocytosis. J. Gen. Virol. 2008, 89 (2), 474-484.
(22) Schaar, H. M. van der, Rust, M. J.; Chen, C.; Ende-Metselaar, H. van der; Wilschut, J.; Zhuang, X.; Smit, J. M. Dissecting the Cell Entry Pathway of Dengue Virus by Single-Particle Tracking in Living Cells. PLOS Pathog. 2008, 4 (12), e1000244.
(23) Modis, Y.; Ogata, S.; Clements, D.; Harrison, S. C. Structure of the Dengue Virus Envelope Protein after Membrane Fusion. Nature 2004, 427 (6972), 313-319.
(24) Bressanelli, S.; Stiasny, K.; Allison, S. L.; Stura, E. A.; Duquerroy, S.; Lescar, J.; Heinz, F. X.; Rey, F. A. Structure of a Flavivirus Envelope Glycoprotein in Its low-pH-induced Membrane Fusion Conformation. EMBO J. 2004, 23 (4), 728-738.
(25) Allison, S. L.; Schalich, J.; Stiasny, K.; Mandl, C. W.; Kunz, C.; Heinz, F. X. Oligomeric Rearrangement of Tick-Bome Encephalitis Virus Envelope Proteins Induced by an Acidic pH. J. Virol. 1995, 69 (2),695-700
(26) Zhou, Z.; Khaliq, M.; Suk, J.-E.; Patkar, C.; Li, L.; Kuhn, R. J.; Post, C. B. Antiviral Compounds Discovered by Virtual Screening of Small-Molecule Libraries against Dengue Virus E Protein. ACS Chem. Biol. 2008, 3 (12), 765-775.
(27) Kampmann, T.; Yennamalli, R.; Campbell, P.; Stoermer, M. J.; Fairlie, D. P.; Kobe, B.; Young, P. R. In Silico Screening of Small Molecule Libraries Using the Dengue Virus Envelope E Protein Has Identified Compounds with Antiviral Activity against Multiple Flaviviruses. Antiviral Res. 2009, 84 (3), 234-241.
(28) Poh, M. K.; Yip, A.; Zhang, S.; Priestle, J. P.; Ma, N. L.; Smit, J. M.; Wilschut, J.; Shi, P.-Y.; Wenk, M. R.; Schul, W. A Small Molecule Fusion Inhibitor of Dengue Virus. Antiviral Res. 2009, 84 (3), 260-266.
(29) Wang, Q.-Y.; Patel, S. J.; Vangrevelinghe, E.; Xu, H. Y.; Rao, R.; Jaber, D.; Schul, W.; Gu, F.; Heudi, O.; Ma, N. L.; Poh, M. K.; Phong, W. Y.; Keller, T. H.; Jacoby, E.; Vasudevan, S. G. A Small-Molecule Dengue Virus Entry Inhibitor. Antimicrob. Agents Chemother. 2009, 53 (5), 1823-1831.
(30) Schmidt, A. G.; Lee, K.; Yang, P. L.; Harrison, S. C. Small-Molecule Inhibitors of Dengue-Virus Entry. PLOS Pathog. 2012, 8 (4), e1002627.
(31) Clark, M. J.; Miduturu, C.; Schmidt, A. G.; Zhu, X.; Pitts, J. D.; Wang, J.; Potisopon, S.; Zhang, J.; Wojciechowski, A.; Hann Chu, J.; Gray, N. S.; Yang, P. L. GNF2 Inhibits Dengue Virus by Targeting Abl Kinases and the Viral E Protein. Cell Chem. Biol. 2016, 23 (4),443-452.
(32) Feng, B. Y.; Shelat, A.; Doman, T. N.; Guy, R. K.; Shoichet, B. K. High-Throughput Assays for Promiscuous Inhibitors. Nat. Chem. Biol. 2005, 1 (3), 146-148.
(33) Irwin, J. J.; Duan, D.; Torosyan, H.; Doak, A. K.; Ziebart, K. T.; Sterling, T.; Tumanian, G.; Shoichet, B. K. An Aggregation Advisor for Ligand Discovery J. Med. Chem. 2015, 58 (17), 7076-7087.
(34) Seidler, J.; McGovern, S. L.; Doman, T. N.; Shoichet, B. K. Identification and Prediction of Promiscuous Aggregating Inhibitors among Known Drugs. J. Med Chem. 2003, 46 (21), 4477-4486.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising,” “including,” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of treating a viral infection comprising administering to a subject in need thereof an effective amount of an antiviral agent, wherein the antiviral agent is:
a compound of the formula: 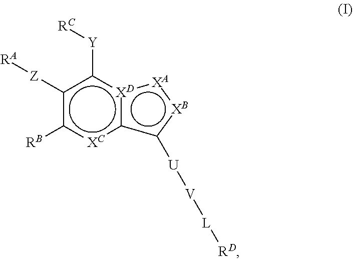
or a pharmaceutically acceptable salt, solvate, hydrate, polymorph, co-crystal, tautomer, stereoisomer, isotopically labeled derivative, or prodrug thereof, wherein:
Z is a bond, O, S, —NR^{E}, or C(R^{F})₂;
R^{E} is hydrogen, substituted or unsubstituted, C₁₋₆, alkyl, or a nitrogen protecting group;
each instance of R^{F} is independently hydrogen, halogen, or substituted or unsubstituted, C₁₋₆ alkyl, or two instances of R^{F} are joined to form ═O;
when Z is a bond or C(R^{F})₂, R^{A} is hydrogen, halogen, substituted or unsubstituted, C₁₋₁₂ alkyl, substituted or unsubstituted C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₁₂ acyl, substituted or unsubstituted C₁₋₁₂ alkyl, substituted or unsubstituted, C₂₋₁₂ alkenyl, substituted or unsubstituted, C₂₋₁₂ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, 3- to 7-membered, monocyclic heterocyclic ring, or substituted or unsubstituted, 5- to 6-membered, monocyclic heteroaryl ring;
when Z is O, S, or NR^{E}, R^{A} is hydrogen, substituted or unsubstituted, C₁₋₁₂ alkyl, substituted or unsubstituted C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, a nitrogen protecting group when attached to a nitrogen atom, an oxygen protecting group when attached to an oxygen atom, or a sulfur protecting group when attached to a sulfur atom;
R^{B} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R′)₂, —NO₂, —NR^{a}C(═O)R^{a}, —NR^{a}C(═O)OR^{a}, —NR^{a}C(═O)N(R)₂, —OC(═O)R^{a}, —OC(═O)OR^{a}, or —OC(═O)N(R^{a})₂;
or R^{A} and R^{B} are joined to form substituted or unsubstituted, 3- to 7-membered, monocyclic carbocyclic ring, substituted or unsubstituted, 3- to 7-membered, monocyclic heterocyclic ring, substituted or unsubstituted, phenyl ring, or substituted or unsubstituted, 5- or 6-membered, monocyclic heteroaryl ring;
Y is C(R^{N})₂, O, S, or NR^{G};
each instance of R^{N} is independently hydrogen, halogen, or substituted or unsubstituted, C₁₋₆alkyl;
R^{G} is hydrogen, substituted or unsubstituted, C₁₋₆alkyl, or a nitrogen protecting group;
R^{C} is hydrogen, substituted or unsubstituted, C₁₋₆ alkyl, a nitrogen protecting group when attached to a nitrogen group, an oxygen protecting group when attached to an oxygen atom, or a sulfur protecting group when attached to a sulfur atom;
or R^{C} and R^{E} are joined to form a substituted or unsubstituted, 3- to 7-membered, monocyclic heterocyclic ring;
or R^{C} and one instance of R^{F} are joined to form a substituted or unsubstituted, 3- to 7-membered, monocyclic heterocyclic ring;
X^{A} is N or NR^{H};
R^{H} is hydrogen, substituted or unsubstituted, C₁₋₆alkyl, or a nitrogen protecting group;
X^{B} is N, NR^{M}, or CR^{J};
R^{M} is hydrogen, substituted or unsubstituted, C₁₋₆ alkyl, or a nitrogen protecting group;
R^{J} is hydrogen, halogen, or substituted or unsubstituted, C₁₋₆ alkyl;
X^{C} is N or CR^{L};
R^{L} is hydrogen, halogen, or substituted or unsubstituted, C₁₋₆ alkyl;
X^{D} is N or C;
provided that 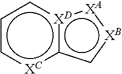
is bicyclic heteroaryl;
—U—V— is —C(═O)—NR^{K}— or —NR^{K}—C(═O)—;
R^{K} is hydrogen, substituted or unsubstituted, C₁₋₆ alkyl, or a nitrogen protecting group;
L is a bond, substituted or unsubstituted, C₁₋₆ alkylene, substituted or unsubstituted, C₂₋₆ alkenylene, or substituted or unsubstituted, C₂₋₆ alkynylene; and
R^{D} is hydrogen, substituted or unsubstituted, C₂₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or R^{D} and R^{K} are joined to form a substituted or unsubstituted, 3- to 7-membered, monocyclic heterocyclic ring, or substituted or unsubstituted, 5- to 6-membered, monocyclic heteroaryl ring:
or a combination thereof, and optionally an additional antiviral agent.

2. A method of preventing a viral infection comprising administering to a subject in need thereof an effective amount of an antiviral agent, wherein the antiviral agent is:
a compound of the formula: 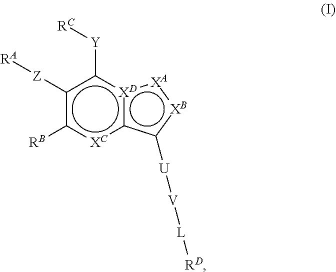
or a pharmaceutically acceptable salt, solvate, hydrate, polymorph, co-crystal, tautomer, stereoisomer, isotopically labeled derivative, or prodrug thereof, wherein:
Z is a bond, O, S, —NR^{E}, or C(R^{F})₂;
R^{E} is hydrogen, substituted or unsubstituted, C₁₋₆ alkyl, or a nitrogen protecting group;
each instance of R^{F} is independently hydrogen, halogen, or substituted or unsubstituted, C₁₋₆ alkyl, or two instances of R^{F} are joined to form ═O;
when Z is a bond or C(R^{F})₂, R^{A} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂, —SR^{a}, —CN, —SCN, —C(═NR^{a})R^{a},—C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₁₂ acyl, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, 3- to 7-membered, monocyclic heterocyclic ring, or substituted or unsubstituted, 5- to 6-membered, monocyclic heteroaryl ring;
when Z is O, S, or NR^{E}, R^{A} is hydrogen, substituted or unsubstituted C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, a nitrogen protecting group when attached to a nitrogen atom, an oxygen protecting group when attached to an oxygen atom, or a sulfur protecting group when attached to a sulfur atom;
R^{B} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl, —OR^{a}, N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or R^{A} and R^{B} are joined to form substituted or unsubstituted, 3- to 7-membered, monocyclic carbocyclic ring, substituted or unsubstituted, 3- to 7-membered, monocyclic heterocyclic ring, substituted or unsubstituted, phenyl ring, or substituted or unsubstituted, 5- or 6-membered, monocyclic heteroaryl ring;
Y is C(R^{N})₂, O, S, or NR^{G};
each instance of R^{N} is independently hydrogen, halogen, or substituted or unsubstituted, C₁₋₆ alkyl;
R^{G} is hydrogen, substituted or unsubstituted, C₁₋₆ alkyl, or a nitrogen protecting group;
R^{C} is hydrogen, substituted or unsubstituted, C₁₋₆ alkyl, a nitrogen protecting group when attached to a nitrogen group, an oxygen protecting group when attached to an oxygen atom, or a sulfur protecting group when attached to a sulfur atom;
or R^{C} and R^{E} are joined to form a substituted or unsubstituted, 3- to 7-membered, monocyclic heterocyclic ring;
or R^{C} and one instance of R^{F} are joined to form a substituted or unsubstituted, 3- to 7-membered, monocyclic heterocyclic ring;
X^{A} is N or NR^{H};
R^{H} is hydrogen, substituted or unsubstituted, C₁₋₆ alkyl, or a nitrogen protecting group;
X^{B} is N, NR^{M}, or CR^{J};
R^{M} is hydrogen, substituted or unsubstituted, C₁₋₆ alkyl, or a nitrogen protecting group;
R^{J} is hydrogen, halogen, or substituted or unsubstituted, C₁₋₆ alkyl;
X^{C} is N or CR^{L};
R^{L} is hydrogen, halogen, or substituted or unsubstituted, C₁₋₆ alkyl;
X^{D} is N or C;
provided that 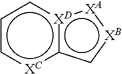
C is bicyclic heteroaryl;
—U—V— is —C(═O)—NR^{K}— or —NR^{K}—C(═O)—;
R^{K} is hydrogen, substituted or unsubstituted, C₁₋₆ alkyl, or a nitrogen protecting group;
L is a bond, substituted or unsubstituted, C₁₋₆ alkylene, substituted or unsubstituted, C₂₋₆ alkenylene, or substituted or unsubstituted, C₂₋₆ alkynylene; and
R^{D} is hydrogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or R^{D} and R^{K} are joined to form a substituted or unsubstituted, 3- to 7-membered, monocyclic heterocyclic ring, or substituted or unsubstituted, 5- to 6-membered, monocyclic heteroaryl ring:
or a combination thereof, and optionally an additional antiviral agent.

3. A method of inhibiting the entry of a virus into a cell comprising contacting the cell with an effective amount of an antiviral agent, wherein the antiviral agent is:
a compound of the formula 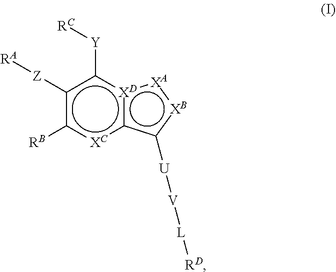
or a pharmaceutically acceptable salt, solvate, hydrate, polymorph, co-crystal, tautomer, stereoisomer, isotopically labeled derivative, or prodrug thereof, wherein:
Z is a bond, O, S, —NR^{E}, or C(R^{F})₂;
R^{E} is hydrogen, substituted or unsubstituted, C₁ alkyl, or a nitrogen protecting group;
each instance of R^{F} is independently hydrogen, halogen, or substituted or unsubstituted, C₁₋₆ alkyl, or two instances of R^{F} are joined to form ═O;
when Z is a bond or C(R^{F})₂, R^{A} is hydrogen, halogen, substituted or unsubstituted, C₁₋₁₂ alkyl, substituted or unsubstituted C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂,
each instance of R^{a} is independently hydrogen, substituted or unsubstituted, C₁₋₁₂ acyl, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, 3- to 7-membered, monocyclic heterocyclic ring, or substituted or unsubstituted, 5- to 6-membered, monocyclic heteroaryl ring;
when Z is O, S, or NR^{E}, R^{A} is hydrogen, substituted or unsubstituted, C₁₋₁₂ alkyl, substituted or unsubstituted C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, a nitrogen protecting group when attached to a nitrogen atom, an oxygen protecting group when attached to an oxygen atom, or a sulfur protecting group when attached to a sulfur atom;
R^{B} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or R^{A} and R^{B} are joined to form substituted or unsubstituted, 3- to 7-membered, monocyclic carbocyclic ring, substituted or unsubstituted, 3- to 7-membered, monocyclic heterocyclic ring, substituted or unsubstituted, phenyl ring, or substituted or unsubstituted, 5- or 6-membered, monocyclic heteroaryl ring;
Y is C(R^{N})₂, O, S, or NR;
each instance of R^{N} is independently hydrogen, halogen, or substituted or unsubstituted, C₁₋₆ alkyl;
R^{G} is hydrogen, substituted or unsubstituted, C₁₋₆ alkyl, or a nitrogen protecting group;
R^{C} is hydrogen, substituted or unsubstituted, C₁₋₆alkyl, a nitrogen protecting group when attached to a nitrogen group, an oxygen protecting group when attached to an oxygen atom, or a sulfur protecting group when attached to a sulfur atom;
or R^{C} and R^{E} are joined to form a substituted or unsubstituted, 3- to 7-membered, monocyclic heterocyclic ring;
or R^{C} and one instance of R^{F} are joined to form a substituted or unsubstituted, 3- to 7-membered, monocyclic heterocyclic ring;
X^{A} is N or NR^{H};
R^{H} is hydrogen, substituted or unsubstituted, C₁₋₆ alkyl, or a nitrogen protecting group;
X^{B} is N, NR^{M}, or CR^{J};
R^{M} is hydrogen, substituted or unsubstituted, C₁₋₆ alkyl, or a nitrogen protecting group;
R^{J} is hydrogen, halogen, or substituted or unsubstituted, C₁₋₆ alkyl;
X^{C} is N or CR^{L};
R^{L} is hydrogen, halogen, or substituted or unsubstituted, C₁₋₆ alkyl;
X^{D} is N or C;
provided that 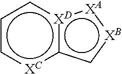
is bicyclic heteroaryl;
—U—V— is —C(═O)—NR^{K}— or —NR^{K}—C(═O)—;
R^{K} is hydrogen, substituted or unsubstituted, C₁₋₆ alkyl, or a nitrogen protecting group;
L is a bond, substituted or unsubstituted, C₁₋₆ alkylene, substituted or unsubstituted, C₂₋₆ alkenylene, or substituted or unsubstituted, C₂₋₆ alkynylene; and
R^{D} is hydrogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or R^{D} and R^{K} are joined to form a substituted or unsubstituted, 3- to 7-membered, monocyclic heterocyclic ring, or substituted or unsubstituted, 5- to 6-membered, monocyclic heteroaryl ring:
or a combination thereof, and optionally an additional antiviral agent.

4. A method of inhibiting an envelope glycoprotein of a virus comprising contacting the virus with an effective amount of an antiviral agent, wherein the antiviral agent is:
a compound of the formula: 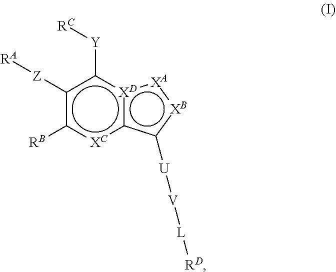
or a pharmaceutically acceptable salt, solvate, hydrate, polymorph, co-crystal, tautomer, stereoisomer, isotopically labeled derivative, or prodrug thereof, wherein:
Z is a bond, O, S, —NR^{E}, or C(R^{F})₂,
R^{E} is hydrogen, substituted or unsubstituted, C₁₋₆ alkyl, or a nitrogen protecting group;
each instance of R^{F} is independently hydrogen, halogen, or substituted or unsubstituted, C₁₋₆ alkyl, or two instances of R^{F} are joined to form ═O;
when Z is a bond or C(R^{F})₂, R^{A} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₁₂ acyl, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, 3- to 7-membered, monocyclic heterocyclic ring, or substituted or unsubstituted, 5- to 6-membered, monocyclic heteroaryl ring;
when Z is O, S, or NR^{E}, R^{A} is hydrogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, a nitrogen protecting group when attached to a nitrogen atom, an oxygen protecting group when attached to an oxygen atom, or a sulfur protecting group when attached to a sulfur atom;
R^{B} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or R^{A} and R^{B} are joined to form substituted or unsubstituted, 3- to 7-membered, monocyclic carbocyclic ring, substituted or unsubstituted, 3- to 7-membered, monocyclic heterocyclic ring, substituted or unsubstituted, phenyl ring, or substituted or unsubstituted, 5- or 6-membered, monocyclic heteroaryl ring;
Y is C(R^{N})₂, O, S, or NR^{G};
each instance of R^{N} is independently hydrogen, halogen, or substituted or unsubstituted, C₁₋₆ alkyl;
R^{G} is hydrogen, substituted or unsubstituted, C₁₋₆ alkyl, or a nitrogen protecting group;
R^{C} is hydrogen, substituted or unsubstituted, C₁₋₆ alkyl, a nitrogen protecting group when attached to a nitrogen group, an oxygen protecting group when attached to an oxygen atom, or a sulfur protecting group when attached to a sulfur atom;
or R^{C} and R^{E} are joined to form a substituted or unsubstituted, 3- to 7-membered, monocyclic heterocyclic ring;
or R^{C} and one instance of R^{F} are joined to form a substituted or unsubstituted, 3- to 7-membered, monocyclic heterocyclic ring;
X^{A} is N or NR^{H};
R^{H} is hydrogen, substituted or unsubstituted, C₁₋₆ alkyl, or a nitrogen protecting group;
X^{B} is N, NR^{M}, or CR^{J};
R^{M} is hydrogen, substituted or unsubstituted, C₁₋₆ alkyl, or a nitrogen protecting group;
R^{J} is hydrogen, halogen, or substituted or unsubstituted, C₁₋₆ alkyl;
X^{C} is N or CR^{L};
R^{L} is hydrogen, halogen, or substituted or unsubstituted, C₁₋₆ f, alkyl;
X^{D} is N or C;
provided that 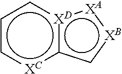
is bicyclic heteroaryl;
—U—V— is —C(═O)—NR^{K}— or —NR^{K}—C(═O)—;
R^{K} is hydrogen, substituted or unsubstituted, C₁₋₆ alkyl, or a nitrogen protecting group;
L is a bond, substituted or unsubstituted, C₁₋₆alkylene, substituted or unsubstituted, C₂₋₆alkenylene, or substituted or unsubstituted, C₂₋₆alkynylene; and
R^{D} is hydrogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R)₂, —OC(═O)R^{a}, —OC(═O)OR^{a}, or —OC(═O)N(R^{a})₂;
or R^{D} and R^{K} are joined to form a substituted or unsubstituted, 3- to 7-membered, monocyclic heterocyclic ring, or substituted or unsubstituted, 5- to 6-membered, monocyclic heteroaryl ring:
or a combination thereof, and optionally an additional antiviral agent.

5. A compound of Formula (I): 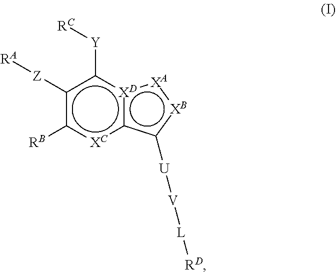
or a pharmaceutically acceptable salt, solvate, hydrate, polymorph, co-crystal, tautomer, stereoisomer, isotopically labeled derivative, or prodrug thereof, wherein:
Z is a bond, O, S, —NR^{E}, or C(R^{F})₂;
R^{E} is hydrogen, substituted or unsubstituted, C₁₋₆, alkyl, or a nitrogen protecting group;
each instance of R^{F} is independently hydrogen, halogen, or substituted or unsubstituted, C₁₋₆ alkyl, or two instances of R^{F} are joined to form ═O;
when Z is a bond or C(R^{F})₂, R^{A} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₁₂ acyl, substituted or unsubstituted C₁₋₁₂ alkyl, substituted or unsubstituted, C₂₋₁₂ alkenyl, substituted or unsubstituted, C₂₋₁₂ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, 3- to 7-membered, monocyclic heterocyclic ring, or substituted or unsubstituted, 5- to 6-membered, monocyclic heteroaryl ring;
when Z is O, S, or NR^{E}, R^{A} is hydrogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, a nitrogen protecting group when attached to a nitrogen atom, an oxygen protecting group when attached to an oxygen atom, or a sulfur protecting group when attached to a sulfur atom;
R^{B} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or R^{A} and R^{B} are joined to form substituted or unsubstituted, 3- to 7-membered, monocyclic carbocyclic ring, substituted or unsubstituted, 3- to 7-membered, monocyclic heterocyclic ring, substituted or unsubstituted, phenyl ring, or substituted or unsubstituted, 5- or 6-membered, monocyclic heteroaryl ring;
Y is C(R^{N})₂, O, S, or NR^{G};
each instance of R^{N} is independently hydrogen, halogen, or substituted or unsubstituted, C₁₋₆ alkyl;
R^{G} is hydrogen, substituted or unsubstituted, C₁₋₆ alkyl, or a nitrogen protecting group;
R^{C} is hydrogen, substituted or unsubstituted, C₁₋₆ alkyl, a nitrogen protecting group when attached to a nitrogen group, an oxygen protecting group when attached to an oxygen atom, or a sulfur protecting group when attached to a sulfur atom;
or R^{C} and R^{E} are joined to form a substituted or unsubstituted, 3- to 7-membered, monocyclic heterocyclic ring;
or R^{C} and one instance of R^{F} are joined to form a substituted or unsubstituted, 3- to 7-membered, monocyclic heterocyclic ring;
X^{A} is N or NR^{H};
R^{H} is hydrogen, substituted or unsubstituted C₁₋₆ alkyl, or a nitrogen protecting group;
X^{B} is N, NR^{M}, or CR^{J};
R^{M} is hydrogen, substituted or unsubstituted, C₁₋₆ alkyl, or a nitrogen protecting group;
R^{J} is hydrogen, halogen, or substituted or unsubstituted, C₁₋₆ alkyl;
X^{C} is N or CR^{L};
R^{L} is hydrogen, halogen, or substituted or unsubstituted, C₁₋₆ alkyl;
X^{D} is N or C;
provided that 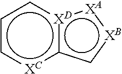
is bicyclic heteroaryl;
—U—V— is —C(═O)—NR^{K}— or —NR^{K}—C(═O)—;
R^{K} is hydrogen, substituted or unsubstituted, C₁₋₆ alkyl, or a nitrogen protecting group;
L is a bond, substituted or unsubstituted, C₁₋₆ alkylene, substituted or unsubstituted, C₂₋₆ alkenylene, or substituted or unsubstituted, C₂₋₆ alkynylene; and
R^{D} is hydrogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
or R^{D} and R are joined to form a substituted or unsubstituted, 3- to 7-membered, monocyclic heterocyclic ring, or substituted or unsubstituted, 5- to 6-membered, monocyclic heteroaryl ring.

6. The method of any one of claims 1-4, or the compound of claim 5, or a pharmaceutically acceptable salt, solvate, hydrate, polymorph, co-crystal, tautomer, stereoisomer, isotopically labeled derivative, or prodrug thereof, wherein:
Z is a bond, O, S, —NR^{E}, or C(R^{F})₂;
R^{E} is hydrogen, substituted or unsubstituted, C₁₋₆, alkyl, or a nitrogen protecting group;
each instance of R^{F} is independently hydrogen, halogen, or substituted or unsubstituted, C₁₋₆ alkyl, or two instances of R^{F} are joined to form ═O;
when Z is a bond or C(R^{F})₂, R^{A} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted, C₁₋₁₂ acyl, substituted or unsubstituted C₁₋₁₂ alkyl, substituted or unsubstituted, C₂₋₁₂ alkenyl, substituted or unsubstituted, C₂₋₁₂ alkynyl, substituted or unsubstituted, 3- to 7-membered, monocyclic carbocyclyl, substituted or unsubstituted, 3- to 7-membered, monocyclic heterocyclyl, substituted or unsubstituted phenyl, substituted or unsubstituted, 5- or 6-membered, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form a substituted or unsubstituted, 3- to 7-membered, monocyclic heterocyclic ring, or substituted or unsubstituted, 5- to 6-membered, monocyclic heteroaryl ring;
when Z is O, S, or NR^{E}, R^{A} is hydrogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, substituted or unsubstituted, 5- to 11-membered, monocyclic or bicyclic heteroaryl, a nitrogen protecting group when attached to a nitrogen atom, an oxygen protecting group when attached to an oxygen atom, or a sulfur protecting group when attached to a sulfur atom;
R^{B} is hydrogen, halogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR)OR, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
provided that at least one of R^{A} and R^{B} is substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl;
Y is O, S, or NR^{G};
R^{G} is hydrogen, substituted or unsubstituted, C₁₋₆ alkyl, or a nitrogen protecting group;
R^{C} is hydrogen, substituted or unsubstituted, C₁₋₆ alkyl, a nitrogen protecting group when attached to a nitrogen group, an oxygen protecting group when attached to an oxygen atom, or a sulfur protecting group when attached to a sulfur atom;
or R^{C} and R^{E} are joined to form a substituted or unsubstituted, 3- to 7-membered, monocyclic heterocyclic ring;
or R^{C} and one instance of R^{F} are joined to form a substituted or unsubstituted, 3- to 7-membered, monocyclic heterocyclic ring;
X^{A} is N or NR^{H};
R^{H} is hydrogen, substituted or unsubstituted, C₁₋₆ alkyl, or a nitrogen protecting group;
X^{B} is N, NR^{M}, or CR^{J};
R^{M} is hydrogen, substituted or unsubstituted, C₁₋₆ alkyl, or a nitrogen protecting group;
R^{J} is hydrogen, halogen, or substituted or unsubstituted, C₁₋₆ alkyl;
X^{C} is N or CR^{L};
R^{L} is hydrogen, halogen, or substituted or unsubstituted, C₁₋₆ alkyl;
X^{D} is N or C;
provided that 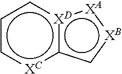
is bicyclic heteroaryl;
—U—V— is —C(═O)—NR^{K}— or —NR^{K}—C(═O)—;
R^{K} is hydrogen, substituted or unsubstituted, C₁₋₆ alkyl, or a nitrogen protecting group;
L is a bond, substituted or unsubstituted, C₁₋₆ alkylene, substituted or unsubstituted, C₂₋₆ alkenylene, or substituted or unsubstituted, C₂₋₆ alkynylene; and
R^{D} is hydrogen, substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl;
provided that the compound is not of the formula:

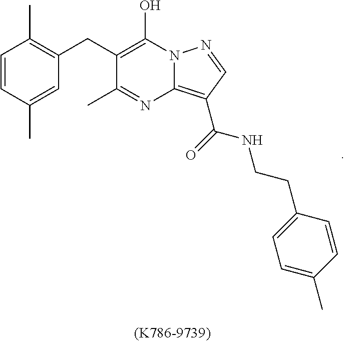 [SMILES: Cc1ccc(CCNC(=O)c2cnn3c(O)c(Cc4cc(C)ccc4C)c(C)nc23)cc1]

7. The method of any one of claims 1-4 and 6, or the compound of any one of claims 5-6, or a pharmaceutically acceptable salt, solvate, hydrate, polymorph, co-crystal, tautomer, stereoisomer, isotopically labeled derivative, or prodrug thereof, wherein the compound is not of the formula:

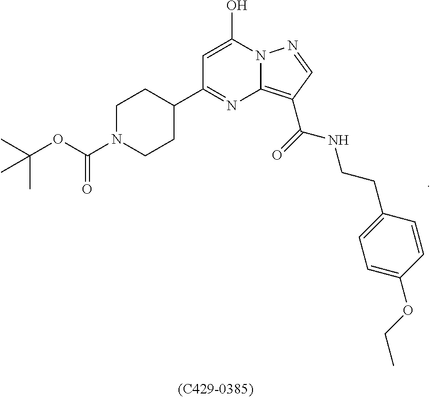 [SMILES: CCOc1ccc(CCNC(=O)c2cnn3c(O)cc(C4CCN(C(=O)OC(C)(C)C)CC4)nc23)cc1]

8. The method of any one of claims 1-4, 6, and 7, or the compound of any one of claims 5-7, or a pharmaceutically acceptable salt, solvate, hydrate, polymorph, co-crystal, tautomer, stereoisomer, isotopically labeled derivative, or prodrug thereof, wherein the compound is not of the formula:

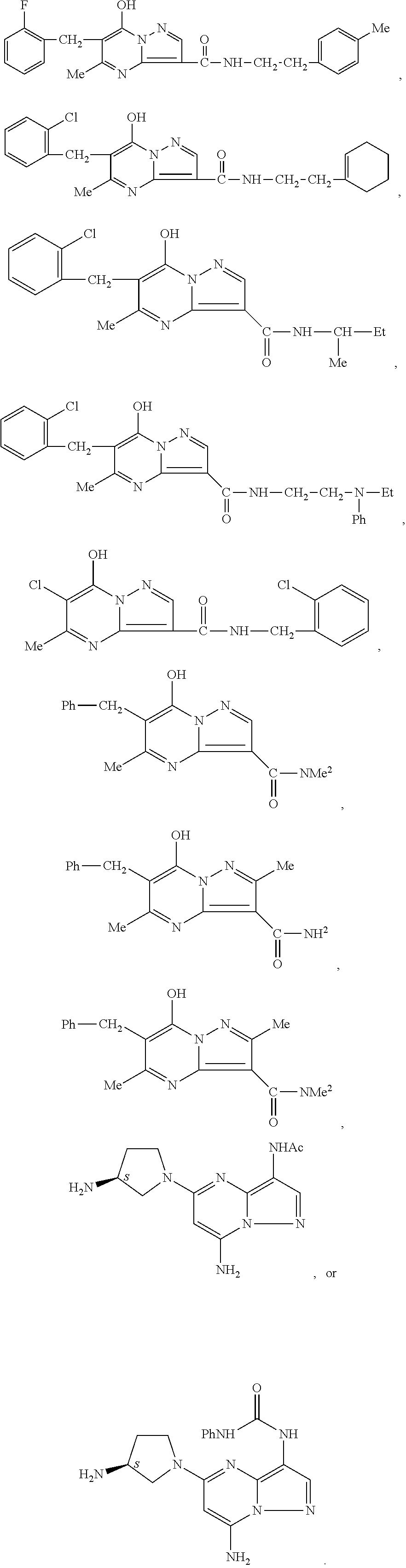

9. The method of any one of claims 1-4 and 6-8, or the compound of any one of claims 5-8, or a pharmaceutically acceptable salt, solvate, hydrate, polymorph, co-crystal, tautomer, stereoisomer, isotopically labeled derivative, or prodrug thereof, wherein the compound is of the formula:

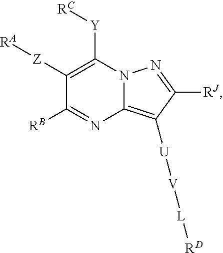

10. The method of any one of claims 1-4 and 6-8, or the compound of any one of claims 5-8, or a pharmaceutically acceptable salt, solvate, hydrate, polymorph, co-crystal, tautomer, stereoisomer, isotopically labeled derivative, or prodrug thereof, wherein the compound is of the formula:

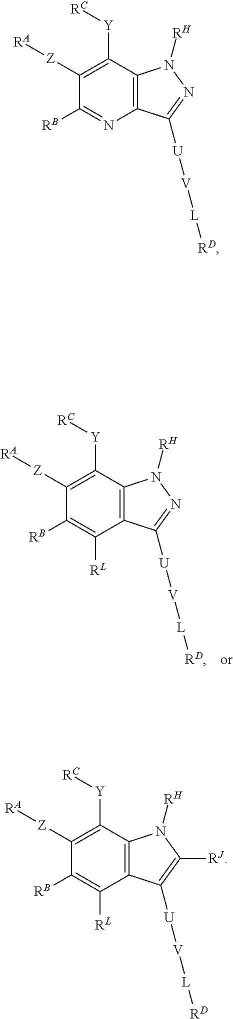

11. The method of any one of claims 1-4 and 6-8, or the compound of any one of claims 5-8, or a pharmaceutically acceptable salt, solvate, hydrate, polymorph, co-crystal, tautomer, stereoisomer, isotopically labeled derivative, or prodrug thereof, wherein the compound is of the formula:

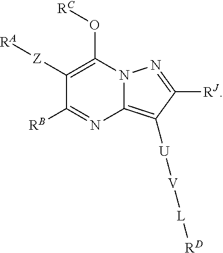

12. The method of any one of claims 1-4 and 6-8, or the compound of any one of claims 5-8, or a pharmaceutically acceptable salt, solvate, hydrate, polymorph, co-crystal, tautomer, stereoisomer, isotopically labeled derivative, or prodrug thereof, wherein the compound is of the formula:

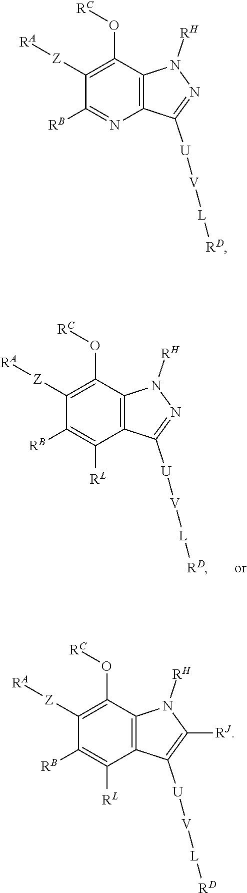

13. The method of any one of claims 1-4 and 6-8, or the compound of any one of claims 5-8, or a pharmaceutically acceptable salt, solvate, hydrate, polymorph, co-crystal, tautomer, stereoisomer, isotopically labeled derivative, or prodrug thereof, wherein the compound is of the formula:

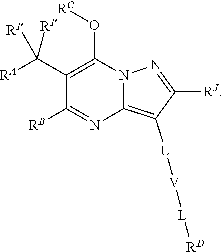

14. The method of any one of claims 1-4 and 6-8, or the compound of any one of claims 5-8, or a pharmaceutically acceptable salt, solvate, hydrate, polymorph, co-crystal, tautomer, stereoisomer, isotopically labeled derivative, or prodrug thereof, wherein the compound is of the formula:

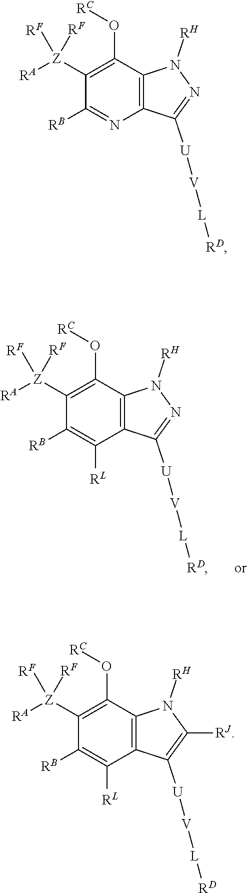

15. The method of any one of claims 1-4 and 6-8, or the compound of any one of claims 5-8, or a pharmaceutically acceptable salt, solvate, hydrate, polymorph, co-crystal, tautomer, stereoisomer, isotopically labeled derivative, or prodrug thereof, wherein the compound is of the formula: 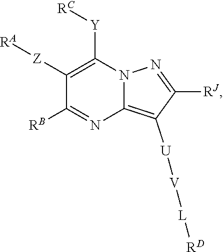
wherein:
R^{B} is hydrogen, halogen, substituted or unsubstituted, C₁₋₆ alkyl, —OR^{a}, —N(R^{a})₂, —SR^{a}, or —CN; and
Y is C(R^{N})₂.

16. The method of any one of claims 1-4 and 6-8, or the compound of any one of claims 5-8, or a pharmaceutically acceptable salt, solvate, hydrate, polymorph, co-crystal, tautomer, stereoisomer, isotopically labeled derivative, or prodrug thereof, wherein the compound is of the formula:

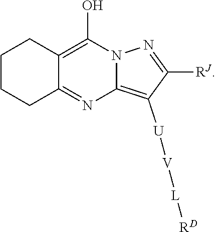

17. The method of any one of claims 1-4 and 6-8, or the compound of any one of claims 5-8, or a pharmaceutically acceptable salt, solvate, hydrate, polymorph, co-crystal, tautomer, stereoisomer, isotopically labeled derivative, or prodrug thereof, wherein the compound is of the formula: 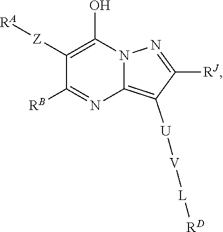
wherein R^{B} is substituted or unsubstituted 4-piperidinyl.

18. The method of any one of claims 1-4 and 6-8, or the compound of any one of claims 5-8, or a pharmaceutically acceptable salt, solvate, hydrate, polymorph, co-crystal, tautomer, stereoisomer isotopically labeled derivative, or prodrug thereof, wherein the compound is of the formula:

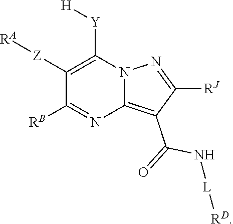

19. The method of any one of claims 1-4 and 6-8, or the compound of any one of claims 5-8, or a pharmaceutically acceptable salt, solvate, hydrate, polymorph, co-crystal, tautomer, stereoisomer isotopically labeled derivative, or prodrug thereof, wherein the compound is of the formula: 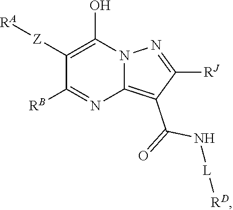
wherein:
R^{B} is hydrogen, halogen, substituted or unsubstituted, C₁₋₆ alkyl, —OR^{a}, —N(R^{a})₂, —SR^{a}, or —CN; and
R^{D} is substituted or unsubstituted phenyl.

20. The method of any one of claims 1-4 and 6-8, or the compound of any one of claims 5-8, or a pharmaceutically acceptable salt, solvate, hydrate, polymorph, co-crystal, tautomer, stereoisomer, isotopically labeled derivative, or prodrug thereof, wherein the compound is of the formula: 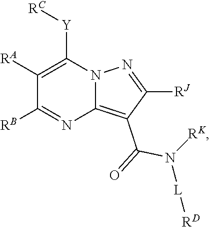
wherein:
R^{A} is hydrogen, halogen, substituted or unsubstituted, C₁₋₆, alkyl, —OR^{a}, —N(R^{a})₂, —SR^{a}, or —CN;
R^{B} is substituted or unsubstituted, C₁₋₁₂ alkyl, substituted or unsubstituted, C₂₋₁₂ alkenyl, substituted or unsubstituted, C₂₋₁₂ alkynyl, substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂; and
Y is O or NR^{G}.

21. The method of any one of claims 1-4 and 6-20, or the compound of any one of claims 5-20, or a pharmaceutically acceptable salt, solvate, hydrate, polymorph, co-crystal, tautomer, stereoisomer, isotopically labeled derivative, or prodrug thereof, wherein Z is a bond.

22. The method of any one of claims 1-4 and 6-20, or the compound of any one of claims 5-20, or a pharmaceutically acceptable salt, solvate, hydrate, polymorph, co-crystal, tautomer, stereoisomer, isotopically labeled derivative, or prodrug thereof, wherein Z is O, S, or NR^{E}.

23. The method of any one of claims 1-4 and 6-20, or the compound of any one of claims 5-20, or a pharmaceutically acceptable salt, solvate, hydrate, polymorph, co-crystal, tautomer, stereoisomer, isotopically labeled derivative, or prodrug thereof, wherein Z is C(R^{F})₂.

24. The method of any one of claims 1-4 and 6-20, or the compound of any one of claims 5-20, or a pharmaceutically acceptable salt, solvate, hydrate, polymorph, co-crystal, tautomer, stereoisomer, isotopically labeled derivative, or prodrug thereof, wherein Z is CH₂.

25. The method of any one of claims 1-4 and 6-20, or the compound of any one of claims 5-20, or a pharmaceutically acceptable salt, solvate, hydrate, polymorph, co-crystal, tautomer, stereoisomer, isotopically labeled derivative, or prodrug thereof, wherein Z is C(═O).

26. The method of any one of claims 1-4 and 6-25, or the compound of any one of claims 5-25, or a pharmaceutically acceptable salt, solvate, hydrate, polymorph, co-crystal, tautomer, stereoisomer, isotopically labeled derivative, or prodrug thereof, wherein R^{E} is hydrogen, or substituted or unsubstituted C₁₋₆ alkyl.

27. The method of any one of claims 1-4 and 6-26, or the compound of any one of claims 5-26, or a pharmaceutically acceptable salt, solvate, hydrate, polymorph, co-crystal, tautomer, stereoisomer, isotopically labeled derivative, or prodrug thereof, wherein each instance of R^{F} is hydrogen.

28. The method of any one of claims 1-4 and 6-26, or the compound of any one of claims 5-26, or a pharmaceutically acceptable salt, solvate, hydrate, polymorph, co-crystal, tautomer, stereoisomer, isotopically labeled derivative, or prodrug thereof, wherein two instances of R^{F} are joined to form ═O.

29. The method of any one of claims 1-4 and 6-28, or the compound of any one of claims 5-28, or a pharmaceutically acceptable salt, solvate, hydrate, polymorph, co-crystal, tautomer, stereoisomer, isotopically labeled derivative, or prodrug thereof, wherein R^{A} is hydrogen.

30. The method of any one of claims 1-4 and 6-28, or the compound of any one of claims 5-28, or a pharmaceutically acceptable salt, solvate, hydrate, polymorph, co-crystal, tautomer, stereoisomer, isotopically labeled derivative, or prodrug thereof, wherein when Z is a bond or C(R^{F})₂, R^{A} is halogen or substituted or unsubstituted, C₁₋₆ alkyl.

31. The method of any one of claims 1-4 and 6-28, or the compound of any one of claims 5-28, or a pharmaceutically acceptable salt, solvate, hydrate, polymorph, co-crystal, tautomer, stereoisomer, isotopically labeled derivative, or prodrug thereof, wherein R^{A} is substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl.

32. The method of any one of claims 1-4 and 6-28, or the compound of any one of claims 5-28, or a pharmaceutically acceptable salt, solvate, hydrate, polymorph, co-crystal, tautomer, stereoisomer, isotopically labeled derivative, or prodrug thereof, wherein R^{A} is substituted or unsubstituted, 3- to 7-membered, monocyclic heterocyclyl.

33. The method of any one of claims 1-4 and 6-28, or the compound of any one of claims 5-28, or a pharmaceutically acceptable salt, solvate, hydrate, polymorph, co-crystal, tautomer, stereoisomer, isotopically labeled derivative, or prodrug thereof, wherein R^{A} is substituted or unsubstituted, 6-membered, monocyclic heterocyclyl.

34. The method of any one of claims 1-4 and 6-28, or the compound of any one of claims 5-28, or a pharmaceutically acceptable salt, solvate, hydrate, polymorph, co-crystal, tautomer, stereoisomer, isotopically labeled derivative, or prodrug thereof, wherein R^{A} is substituted or unsubstituted piperazinyl.

35. The method of any one of claims 1-4 and 6-28, or the compound of any one of claims 5-28, or a pharmaceutically acceptable salt, solvate, hydrate, polymorph, co-crystal, tautomer, stereoisomer, isotopically labeled derivative, or prodrug thereof, wherein R^{A} is substituted or unsubstituted phenyl.

36. The method of any one of claims 1-4 and 6-28, or the compound of any one of claims 5-28, or a pharmaceutically acceptable salt, solvate, hydrate, polymorph, co-crystal, tautomer, stereoisomer, isotopically labeled derivative, or prodrug thereof, wherein R^{A} is ortho-substituted phenyl, meta-substituted phenyl, para-substituted phenyl, ortho, ortho-substituted phenyl, ortho, meta-substituted phenyl, ortho, para-substituted phenyl, meta, meta-substituted phenyl, or meta, para-substituted phenyl.

37. The method of any one of claims 1-4 and 6-28, or the compound of any one of claims 5-28, or a pharmaceutically acceptable salt, solvate, hydrate, polymorph, co-crystal, tautomer, stereoisomer, isotopically labeled derivative, or prodrug thereof, wherein R^{A} is of the formula: 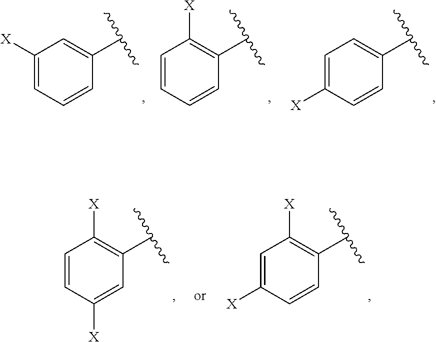
wherein each instance of X is independently hydrogen, halogen, substituted or unsubstituted, C₁₋₆; alkyl, —OR^{a}, —N(R^{a})₂, —SR^{a}, or —CN.

38. The method of any one of claims 1-4 and 6-28, or the compound of any one of claims 5-28, or a pharmaceutically acceptable salt, solvate, hydrate, polymorph, co-crystal, tautomer, stereoisomer, isotopically labeled derivative, or prodrug thereof, wherein R^{A} is of the formula:

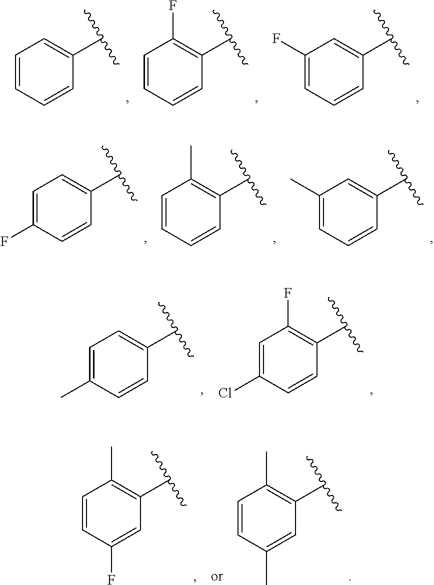

39. The method of any one of claims 1-4 and 6-28, or the compound of any one of claims 5-28, or a pharmaceutically acceptable salt, solvate, hydrate, polymorph, co-crystal, tautomer, stereoisomer, isotopically labeled derivative, or prodrug thereof, wherein R^{A} is of the formula: 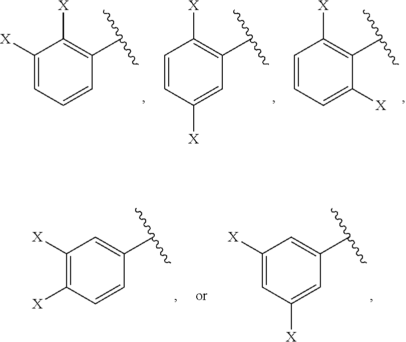
wherein each instance of X is independently hydrogen, halogen, substituted or unsubstituted, C₁₋₆alkyl, —OR^{a}, —N(R^{a})₂, —SR^{a}, or —CN.

40. The method of any one of claims 1-4 and 6-39, or the compound of any one of claims 5-39, or a pharmaceutically acceptable salt, solvate, hydrate, polymorph, co-crystal, tautomer, stereoisomer, isotopically labeled derivative, or prodrug thereof, wherein each instance of R^{a} is hydrogen.

41. The method of any one of claims 1-4 and 6-28, or the compound of any one of claims 5-28, or a pharmaceutically acceptable salt, solvate, hydrate, polymorph, co-crystal, tautomer, stereoisomer, isotopically labeled derivative, or prodrug thereof, wherein —Z—R^{A} is hydrogen, halogen, or substituted or unsubstituted, C₁₋₁₂ alkyl.

42. The method of any one of claims 1-4 and 6-41, or the compound of any one of claims 5-41, or a pharmaceutically acceptable salt, solvate, hydrate, polymorph, co-crystal, tautomer, stereoisomer, isotopically labeled derivative, or prodrug thereof, wherein R^{B} is hydrogen, halogen, or substituted or unsubstituted C₁₋₁₂ alkyl.

43. The method of any one of claims 1-4 and 6-41, or the compound of any one of claims 5-41, or a pharmaceutically acceptable salt, solvate, hydrate, polymorph, co-crystal, tautomer, stereoisomer, isotopically labeled derivative, or prodrug thereof, wherein R^{B} is hydrogen.

44. The method of any one of claims 1-4 and 6-41, or the compound of any one of claims 5-41, or a pharmaceutically acceptable salt, solvate, hydrate, polymorph, co-crystal, tautomer, stereoisomer, isotopically labeled derivative, or prodrug thereof, wherein R^{B} is substituted or unsubstituted, C₁₋₆ alkyl.

45. The method of any one of claims 1-4 and 6-41, or the compound of any one of claims 5-41, or a pharmaceutically acceptable salt, solvate, hydrate, polymorph, co-crystal, tautomer, stereoisomer, isotopically labeled derivative, or prodrug thereof, wherein R^{B} is —CH₃.

46. The method of any one of claims 1-4 and 6-41, or the compound of any one of claims 5-41, or a pharmaceutically acceptable salt, solvate, hydrate, polymorph, co-crystal, tautomer, stereoisomer, isotopically labeled derivative, or prodrug thereof, wherein R^{B} is substituted or unsubstituted, 3- to 13-membered, monocyclic or bicyclic carbocyclyl, substituted or unsubstituted, 3- to 13-membered, monocyclic or bicyclic heterocyclyl, substituted or unsubstituted, 6- to 11-membered, monocyclic or bicyclic aryl, or substituted or unsubstituted, 5- to 11-membered, monocyclic or bicyclic heteroaryl.

47. The method of any one of claims 1-4 and 6-41, or the compound of any one of claims 5-41, or a pharmaceutically acceptable salt, solvate, hydrate, polymorph, co-crystal, tautomer, stereoisomer, isotopically labeled derivative, or prodrug thereof, wherein R^{B} is substituted or unsubstituted, 6- to 11-membered, monocyclic or bicyclic aryl, or substituted or unsubstituted, 5- to 11-membered, monocyclic or bicyclic heteroaryl.

48. The method of any one of claims 1-4 and 6-41, or the compound of any one of claims 5-41, or a pharmaceutically acceptable salt, solvate, hydrate, polymorph, co-crystal, tautomer, stereoisomer, isotopically labeled derivative, or prodrug thereof, wherein R^{B} is substituted or unsubstituted phenyl.

49. The method of any one of claims 1-4 and 6-41, or the compound of any one of claims 5-41, or a pharmaceutically acceptable salt, solvate, hydrate, polymorph, co-crystal, tautomer, stereoisomer, isotopically labeled derivative, or prodrug thereof, wherein R^{A} and R^{B} are joined to form substituted or unsubstituted, 3- to 7-membered, monocyclic carbocyclic ring, substituted or unsubstituted, 3- to 7-membered, monocyclic heterocyclic ring, substituted or unsubstituted, phenyl ring, or substituted or unsubstituted, 5- or 6-membered, monocyclic heteroaryl ring.

50. The method of any one of claims 1-4 and 6-49, or the compound of any one of claims 5-49, or a pharmaceutically acceptable salt, solvate, hydrate, polymorph, co-crystal, tautomer, stereoisomer, isotopically labeled derivative, or prodrug thereof, wherein Y is C(R^{N})₂.

51. The method of any one of claims 1-4 and 6-49, or the compound of any one of claims 5-49, or a pharmaceutically acceptable salt, solvate, hydrate, polymorph, co-crystal, tautomer, stereoisomer, isotopically labeled derivative, or prodrug thereof, wherein Y is O.

52. The method of any one of claims 1-4 and 6-49, or the compound of any one of claims 5-49, or a pharmaceutically acceptable salt, solvate, hydrate, polymorph, co-crystal, tautomer, stereoisomer, isotopically labeled derivative, or prodrug thereof, wherein Y is NR^{G}.

53. The method of any one of claims 1-4 and 6-52, or the compound of any one of claims 5-52, or a pharmaceutically acceptable salt, solvate, hydrate, polymorph, co-crystal, tautomer, stereoisomer, isotopically labeled derivative, or prodrug thereof, wherein R^{G} is hydrogen.

54. The method of any one of claims 1-4 and 6-53, or the compound of any one of claims 5-53, or a pharmaceutically acceptable salt, solvate, hydrate, polymorph, co-crystal, tautomer, stereoisomer, isotopically labeled derivative, or prodrug thereof, wherein R^{C} is hydrogen.

55. The method of any one of claims 1-4 and 6-54, or the compound of any one of claims 5-54, or a pharmaceutically acceptable salt, solvate, hydrate, polymorph, co-crystal, tautomer, stereoisomer, isotopically labeled derivative, or prodrug thereof, wherein R^{C} and R^{E} are joined to form a substituted or unsubstituted, 3- to 7-membered, monocyclic heterocyclic ring.

56. The method of any one of claims 1-4 and 6-55, or the compound of any one of claims 5-55, or a pharmaceutically acceptable salt, solvate, hydrate, polymorph, co-crystal, tautomer, stereoisomer, isotopically labeled derivative, or prodrug thereof, wherein X^{A} is N.

57. The method of any one of claims 1-4 and 6-56, or the compound of any one of claims 5-56, or a pharmaceutically acceptable salt, solvate, hydrate, polymorph, co-crystal, tautomer, stereoisomer, isotopically labeled derivative, or prodrug thereof, wherein X^{A} is NR^{H}.

58. The method of any one of claims 1-4 and 6-57, or the compound of any one of claims 5-57, or a pharmaceutically acceptable salt, solvate, hydrate, polymorph, co-crystal, tautomer, stereoisomer, isotopically labeled derivative, or prodrug thereof, wherein R^{H} is hydrogen, or substituted or unsubstituted, C₁₋₆ alkyl.

59. The method of any one of claims 1-4 and 6-58, or the compound of any one of claims 5-58, or a pharmaceutically acceptable salt, solvate, hydrate, polymorph, co-crystal, tautomer, stereoisomer, isotopically labeled derivative, or prodrug thereof, wherein X^{B} is N.

60. The method of any one of claims 1-4 and 6-59, or the compound of any one of claims 5-59, or a pharmaceutically acceptable salt, solvate, hydrate, polymorph, co-crystal, tautomer, stereoisomer, isotopically labeled derivative, or prodrug thereof, wherein X^{B} is CR^{J}.

61. The method of any one of claims 1-4 and 6-59, or the compound of any one of claims 5-59, or a pharmaceutically acceptable salt, solvate, hydrate, polymorph, co-crystal, tautomer, stereoisomer, isotopically labeled derivative, or prodrug thereof, wherein X^{B} is CH.

62. The method of any one of claims 1-4 and 6-61, or the compound of any one of claims 5-61, or a pharmaceutically acceptable salt, solvate, hydrate, polymorph, co-crystal, tautomer, stereoisomer, isotopically labeled derivative, or prodrug thereof, wherein R^{J} is hydrogen or halogen.

63. The method of any one of claims 1-4 and 6-62, or the compound of any one of claims 5-62, or a pharmaceutically acceptable salt, solvate, hydrate, polymorph, co-crystal, tautomer, stereoisomer, isotopically labeled derivative, or prodrug thereof, wherein X^{C} is N.

64. The method of any one of claims 1-4 and 6-63, or the compound of any one of claims 5-63, or a pharmaceutically acceptable salt, solvate, hydrate, polymorph, co-crystal, tautomer, stereoisomer, isotopically labeled derivative, or prodrug thereof, wherein X^{C} is CR^{L}.

65. The method of any one of claims 1-4 and 6-63, or the compound of any one of claims 5-63, or a pharmaceutically acceptable salt, solvate, hydrate, polymorph, co-crystal, tautomer, stereoisomer, isotopically labeled derivative, or prodrug thereof, wherein X^{C} is CH.

66. The method of any one of claims 1-4 and 6-65, or the compound of any one of claims 5-65, or a pharmaceutically acceptable salt, solvate, hydrate, polymorph, co-crystal, tautomer, stereoisomer, isotopically labeled derivative, or prodrug thereof, wherein R^{L} is hydrogen or halogen.

67. The method of any one of claims 1-4 and 6-66, or the compound of any one of claims 5-66, or a pharmaceutically acceptable salt, solvate, hydrate, polymorph, co-crystal, tautomer, stereoisomer, isotopically labeled derivative, or prodrug thereof, wherein X^{D} is N.

68. The method of any one of claims 1-4 and 6-67, or the compound of any one of claims 5-67, or a pharmaceutically acceptable salt, solvate, hydrate, polymorph, co-crystal, tautomer, stereoisomer, isotopically labeled derivative, or prodrug thereof, wherein X^{D} is C.

69. The method of any one of claims 1-4 and 6-68, or the compound of any one of claims 5-68, or a pharmaceutically acceptable salt, solvate, hydrate, polymorph, co-crystal, tautomer, stereoisomer, isotopically labeled derivative, or prodrug thereof, wherein —U—V— is —C(═O)—NH—.

70. The method of any one of claims 1-4 and 6-68, or the compound of any one of claims 5-68, or a pharmaceutically acceptable salt, solvate, hydrate, polymorph, co-crystal, tautomer, stereoisomer, isotopically labeled derivative, or prodrug thereof, wherein —U—V— is —NH—C(═O)—.

71. The method of any one of claims 1-4 and 6-68, or the compound of any one of claims 5-68, or a pharmaceutically acceptable salt, solvate, hydrate, polymorph, co-crystal, tautomer, stereoisomer, isotopically labeled derivative, or prodrug thereof, wherein R^{K} is substituted or unsubstituted, C alkyl.

72. The method of any one of claims 1-4 and 6-71, or the compound of any one of claims 5-71, or a pharmaceutically acceptable salt, solvate, hydrate, polymorph, co-crystal, tautomer, stereoisomer, isotopically labeled derivative, or prodrug thereof, wherein L is a bond.

73. The method of any one of claims 1-4 and 6-71, or the compound of any one of claims 5-71, or a pharmaceutically acceptable salt, solvate, hydrate, polymorph, co-crystal, tautomer, stereoisomer, isotopically labeled derivative, or prodrug thereof, wherein L is substituted or unsubstituted, C₁₋₆ alkylene, substituted or unsubstituted, C₂₋₆ alkenylene, or substituted or unsubstituted, C₂₋₆ alkynylene.

74. The method of any one of claims 1-4 and 6-71, or the compound of any one of claims 5-71, or a pharmaceutically acceptable salt, solvate, hydrate, polymorph, co-crystal, tautomer, stereoisomer, isotopically labeled derivative, or prodrug thereof, wherein L is —CH₂—, —CH₂—CH₂—, or —(CH₂)₃—.

75. The method of any one of claims 1-4 and 6-74, or the compound of any one of claims 5-74, or a pharmaceutically acceptable salt, solvate, hydrate, polymorph, co-crystal, tautomer, stereoisomer, isotopically labeled derivative, or prodrug thereof, wherein R^{D} is hydrogen.

76. The method of any one of claims 1-4 and 6-74, or the compound of any one of claims 5-74, or a pharmaceutically acceptable salt, solvate, hydrate, polymorph, co-crystal, tautomer, stereoisomer, isotopically labeled derivative, or prodrug thereof, wherein R^{D} is substituted or unsubstituted, 3- to 13-membered, monocyclic or bicyclic heterocyclyl.

77. The method of any one of claims 1-4 and 6-74, or the compound of any one of claims 5-74, or a pharmaceutically acceptable salt, solvate, hydrate, polymorph, co-crystal, tautomer, stereoisomer, isotopically labeled derivative, or prodrug thereof, wherein R^{D} is substituted or unsubstituted, 6-membered, monocyclic heterocyclyl.

78. The method of any one of claims 1-4 and 6-74, or the compound of any one of claims 5-74, or a pharmaceutically acceptable salt, solvate, hydrate, polymorph, co-crystal, tautomer, stereoisomer, isotopically labeled derivative, or prodrug thereof, wherein R^{D} is substituted or unsubstituted piperidinyl or substituted or unsubstituted piperazinyl.

79. The method of any one of claims 1-4 and 6-74, or the compound of any one of claims 5-74, or a pharmaceutically acceptable salt, solvate, hydrate, polymorph, co-crystal, tautomer, stereoisomer, isotopically labeled derivative, or prodrug thereof, wherein R^{D} is substituted or unsubstituted, 6- to 11-membered, monocyclic or bicyclic aryl.

80. The method of any one of claims 1-4 and 6-74, or the compound of any one of claims 5-74, or a pharmaceutically acceptable salt, solvate, hydrate, polymorph, co-crystal, tautomer, stereoisomer, isotopically labeled derivative, or prodrug thereof, wherein R^{D} is substituted or unsubstituted phenyl.

81. The method of any one of claims 1-4 and 6-74, or the compound of any one of claims 5-74, or a pharmaceutically acceptable salt, solvate, hydrate, polymorph, co-crystal, tautomer, stereoisomer, isotopically labeled derivative, or prodrug thereof, wherein R^{D} is ortho-substituted phenyl, meta-substituted phenyl, para-substituted phenyl, ortho, ortho-substituted phenyl, ortho, meta-substituted phenyl, ortho, para-substituted phenyl, meta, meta-substituted phenyl, or meta, para-substituted phenyl.

82. The method of any one of claims 1-4 and 6-74, or the compound of any one of claims 5-74, or a pharmaceutically acceptable salt, solvate, hydrate, polymorph, co-crystal, tautomer, stereoisomer, isotopically labeled derivative, or prodrug thereof, wherein R^{D} is substituted or unsubstituted, 5- to 11-membered, monocyclic or bicyclic heteroaryl.

83. The method of any one of claims 1-4 and 6-74, or the compound of any one of claims 5-74, or a pharmaceutically acceptable salt, solvate, hydrate, polymorph, co-crystal, tautomer, stereoisomer, isotopically labeled derivative, or prodrug thereof, wherein R^{D} is substituted or unsubstituted, 5- to 6-membered, monocyclic heteroaryl.

84. The method of any one of claims 1-4 and 6-74, or the compound of any one of claims 5-74, or a pharmaceutically acceptable salt, solvate, hydrate, polymorph, co-crystal, tautomer, stereoisomer, isotopically labeled derivative, or prodrug thereof, wherein R^{D} is substituted or unsubstituted pyridinyl.

85. The method of any one of claims 1-4 and 6-74, or the compound of any one of claims 5-74, or a pharmaceutically acceptable salt, solvate, hydrate, polymorph, co-crystal, tautomer, stereoisomer, isotopically labeled derivative, or prodrug thereof, wherein R^{D} is of the formula: 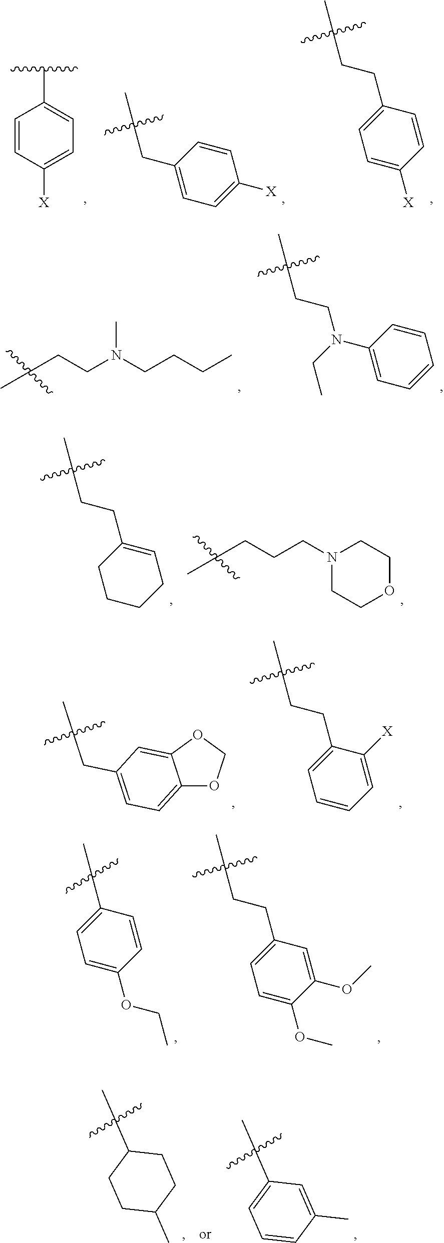
wherein X is hydrogen, halogen, substituted or unsubstituted, C₁₋₆ alkyl, —OR^{a}, —N(R^{a})₂, —SR^{a}, or —CN.

86. The method of any one of claims 1-4, or the compound of claim 5, or a pharmaceutically acceptable salt, solvate, hydrate, polymorph, co-crystal, tautomer, stereoisomer, isotopically labeled derivative, or prodrug thereof, wherein the compound is of the formula:

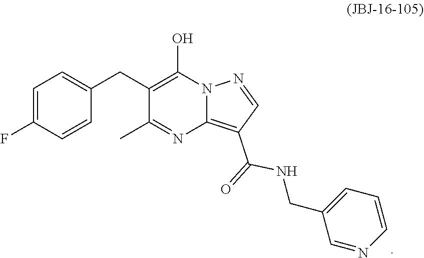 [SMILES: Cc1nc2c(C(=O)NCc3cccnc3)cnn2c(O)c1Cc1ccc(F)cc1]

87. The method of any one of claims 1-4, or the compound of claim 5, or a pharmaceutically acceptable salt, solvate, hydrate, polymorph, co-crystal, tautomer, stereoisomer, isotopically labeled derivative, or prodrug thereof, wherein the compound is of the formula:

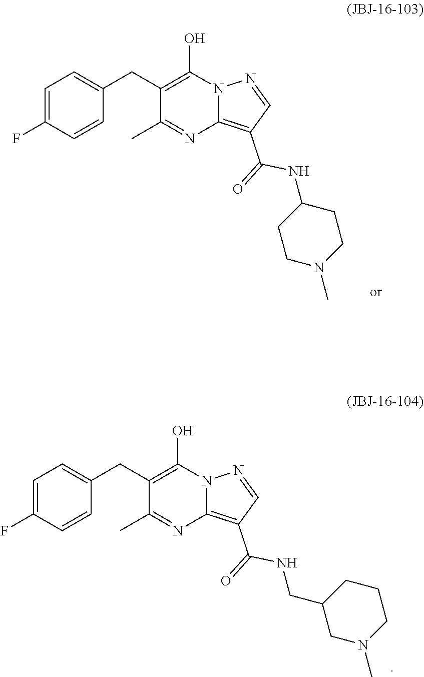 [SMILES: Cc1nc2c(C(=O)NC3CCN(C)CC3)cnn2c(O)c1Cc1ccc(F)cc1 ; Cc1nc2c(C(=O)NCC3CCCN(C)C3)cnn2c(O)c1Cc1ccc(F)cc1]

88. The method of any one of claims 1-4, or the compound of claim 5, or a pharmaceutically acceptable salt, solvate, hydrate, polymorph, co-crystal, tautomer, stereoisomer, isotopically labeled derivative, or prodrug thereof, wherein the compound is of the formula:

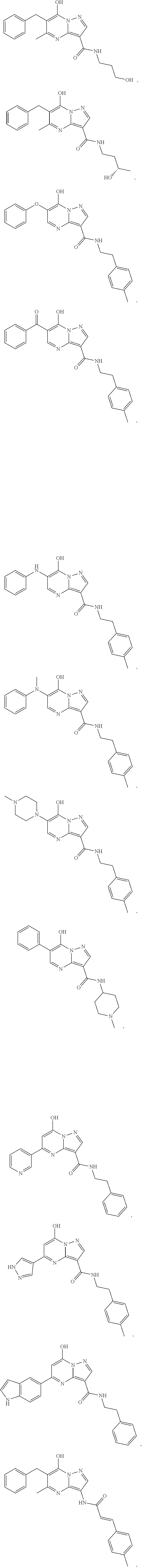

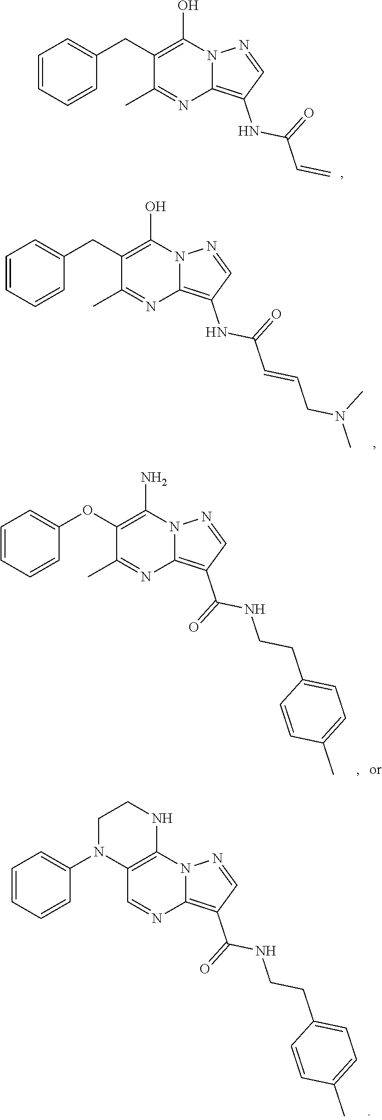 [SMILES: C=CC(=O)Nc1cnn2c(O)c(Cc3ccccc3)c(C)nc12 ; Cc1nc2c(NC(=O)/C=C/CN(C)C)cnn2c(O)c1Cc1ccccc1 ; Cc1ccc(CCNC(=O)c2cnn3c(N)c(Oc4ccccc4)c(C)nc23)cc1 ; Cc1ccc(CCNC(=O)c2cnn3c4c(cnc23)N(c2ccccc2)CCN4)cc1]

89. The method of any one of claims 1-4, or the compound of claim 5, or a pharmaceutically acceptable salt, solvate, hydrate, polymorph, co-crystal, tautomer, stereoisomer isotopically labeled derivative, or prodrug thereof, wherein the compound is of the formula:

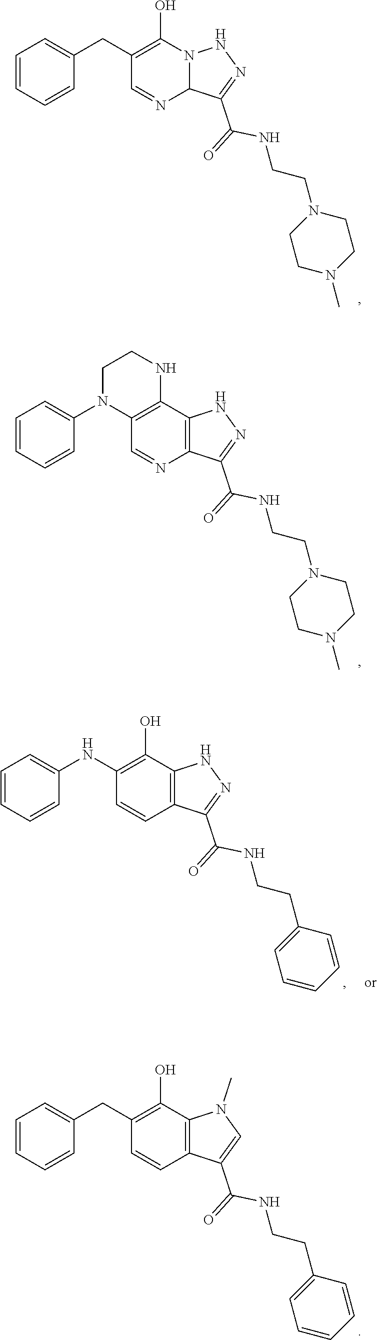 [SMILES: CN1CCN(CCNC(=O)C2=NNN3C(O)=C(Cc4ccccc4)C=NC23)CC1 ; CN1CCN(CCNC(=O)c2n[nH]c3c4c(cnc23)N(c2ccccc2)CCN4)CC1 ; O=C(NCCc1ccccc1)c1n[nH]c2c(O)c(Nc3ccccc3)ccc12 ; Cn1cc(C(=O)NCCc2ccccc2)c2ccc(Cc3ccccc3)c(O)c21]

90. The method of any one of claim 14, or the compound of claim 5, or a pharmaceutically acceptable salt, solvate, hydrate, polymorph, co-crystal, tautomer, stereoisomer isotopically labeled derivative, or prodrug thereof, wherein the compound is of the formula:

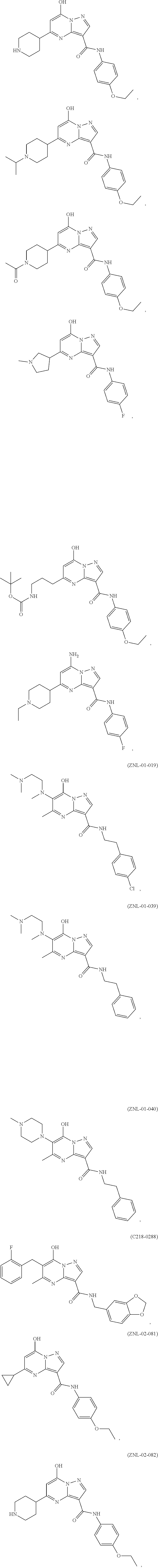

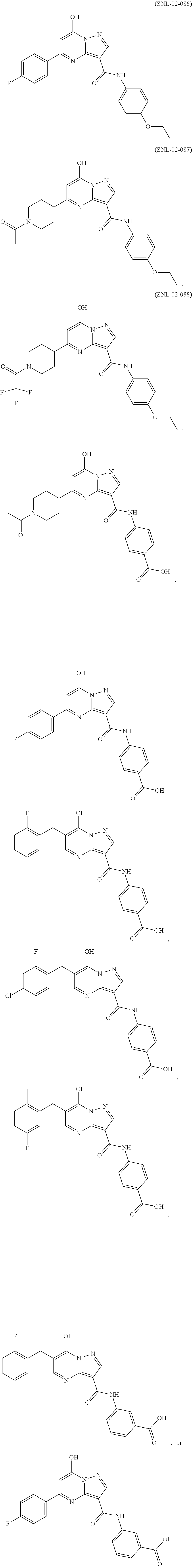

91. The compound of any one of claims 5-90, or a pharmaceutically acceptable salt thereof.

92. A pharmaceutical composition comprising:
a compound of any one of claims 5-90, or a pharmaceutically acceptable salt, solvate, hydrate, polymorph, co-crystal, tautomer, stereoisomer, isotopically labeled derivative, or prodrug thereof; and
optionally a pharmaceutically acceptable excipient.

93. The pharmaceutical composition of claim 92 further comprising an additional pharmaceutical agent.

94. The pharmaceutical composition of claim 93, wherein the additional pharmaceutical agent is an additional antiviral agent.

95. A kit comprising:
a compound of any one of claims 5-90, or a pharmaceutically acceptable salt, solvate, hydrate, polymorph, co-crystal, tautomer, stereoisomer, isotopically labeled derivative, or prodrug thereof, or a pharmaceutical composition of any one of claims 92-94; and
instructions for using the compound, or a pharmaceutically acceptable salt, solvate, hydrate, polymorph, co-crystal, tautomer, stereoisomer, isotopically labeled derivative, or prodrug thereof, or the pharmaceutical composition.

96. A method of treating a viral infection comprising administering to a subject in need thereof an effective amount of an antiviral agent, wherein the antiviral agent is:
a compound of any one of claims 5-90, or a pharmaceutically acceptable salt, solvate, hydrate, polymorph, co-crystal, tautomer, stereoisomer, isotopically labeled derivative, or prodrug thereof; or
a compound of the formula: 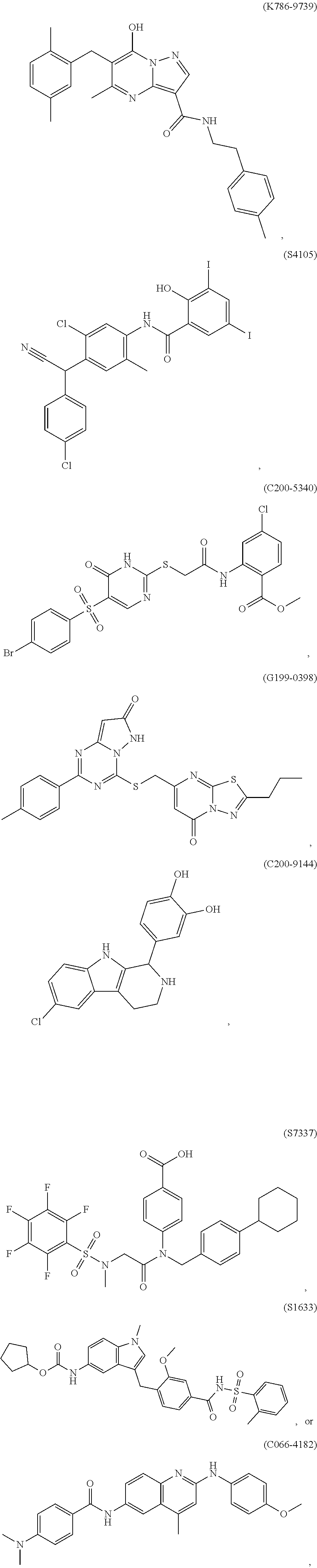
viral infection;
or a combination thereof, and optionally an additional antiviral agent.

97. A method of preventing a viral infection comprising administering to a subject in need thereof an effective amount of an antiviral agent, wherein the antiviral agent is:
a compound of any one of claims 5-90, or a pharmaceutically acceptable salt, solvate, hydrate, polymorph, co-crystal, tautomer, stereoisomer, isotopically labeled derivative, or prodrug thereof; or
a compound of the formula: 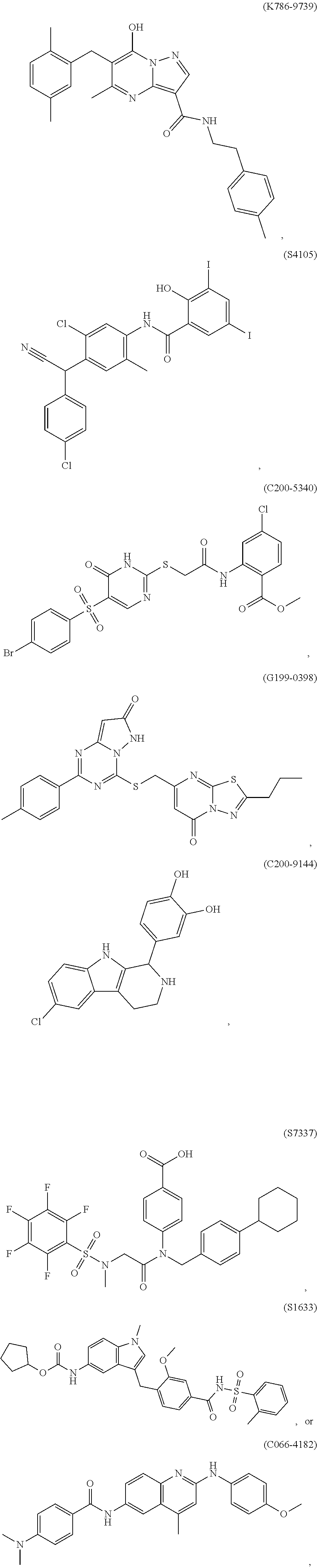
or a pharmaceutically acceptable salt, solvate, hydrate, polymorph, co-crystal, tautomer, stereoisomer, derivative, or prodrug thereof;
or a combination thereof, and optionally an additional antiviral agent.

98. The method of any one of claims 1-4, 6-90, and 96-97, wherein the effective amount is effective in inhibiting the entry of the virus into a cell of the subject.

99. The method of any one of claims 1-4, 6-90, and 96-98, wherein the viral infection is Dengue fever.

100. The method of any one of claims 1-4, 6-90, and 96-98, wherein the viral infection is Dengue hemorrhagic fever (DHF) or Dengue shock syndrome (DSS).

101. The method of any one of claims 1-4, 6-90, and 96-98, wherein the viral infection is yellow fever, West Nile encephalitis, West Nile fever, Japanese encephalitis, or Zika fever, preferably, Zika fever.

102. The method of any one of claims 1-4, 6-90, and 96-98, wherein the viral infection is hepatitis B, hepatitis C, fulminant viral hepatitis, severe acute respiratory syndrome (SARS), viral myocarditis, influenza A virus infection, influenza B virus infection, parainfluenza virus infection, measles virus infection, vesicular stomatitis virus infection, rabies virus infection, Ebola virus infection, Junin virus infection, human cytomegalovirus infection, herpes simplex virus 1 infection, poliovirus infection, Marburg virus infection, Lassa fever virus infection, Venezuelan equine encephalitis, Rift Valley fever virus infection, Korean hemorrhagic fever virus infection, Crimean-Congo hemorrhagic fever virus infection, human immunodeficiency virus (HIV) infection, Saint Louise encephalitis, Kyasanur Forest disease, Murray Valley encephalitis, tick-borne encephalitis, Theiler's disease, hepatocellular carcinoma, Kyasanur Forest disease (KFD), Alkhurma disease, Omsk hemorrhagic fever, Rocio encephalitis, wesselsbron disease, Powassan diseas, Israeli turkey meningoencephalitis, Central European tickbome fever, Louping ill, California encephalitis, Border disease, bovine viral diarrhea-mucosal disease, classical swine fever, or bovine hemorrhagic syndrome.

103. The method of any one of claims 1-4, 6-90, and 96-102, wherein the subject is a mammal.

104. The method of any one of claims 1-4, 6-90, and 96-102, wherein the subject is a human.

105. A method of inhibiting the entry of a virus into a cell comprising contacting the cell with an effective amount of an antiviral agent, wherein the antiviral agent is:
a compound of any one of claims 5-90, or a pharmaceutically acceptable salt, solvate, hydrate, polymorph, co-crystal, tautomer, stereoisomer, isotopically labeled derivative, or prodrug thereof; or
a compound of the formula: 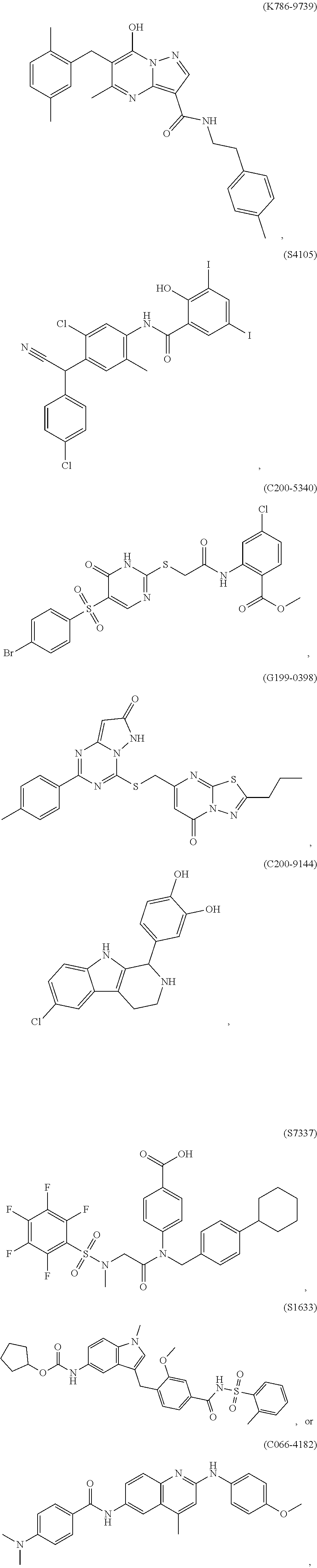
or a pharmaceutically acceptable salt, solvate, hydrate, polymorph, co-crystal, tautomer, stereoisomer, derivative, or prodrug thereof;
or a combination thereof, and optionally an additional antiviral agent.

106. The method of any one of claims 1-4, 6-90, and 96-105, wherein the effective amount is effective in inhibiting an envelope glycoprotein of the virus.

107. The method of any one of claims 1-4, 6-90, and 96-106, wherein the effective amount is effective in inhibiting the fusion between the envelope of the virus and the membrane of the cell.

108. The method of any one of claims 1-4, 6-90, and 96-107, wherein the cell is in vitro.

109. A method of inhibiting an envelope glycoprotein of a virus comprising contacting the virus with an effective amount of an antiviral agent, wherein the antiviral agent is:
a compound of any one of claims 5-90, or a pharmaceutically acceptable salt, solvate, hydrate, polymorph co-crystal, tautomer, stereoisomer, isotopically labeled derivative, or prodrug thereof; or
a compound of the formula: 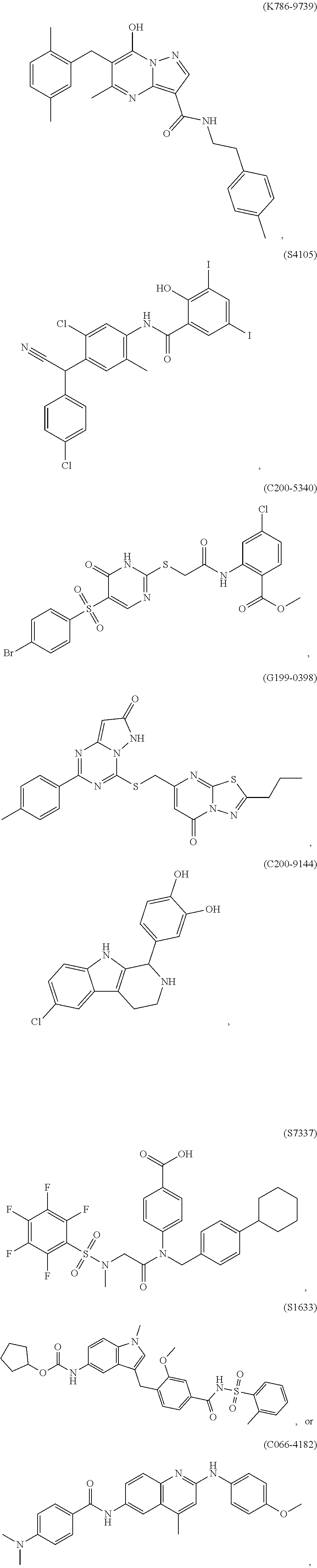
or a pharmaceutically acceptable salt, solvate, hydrate, polymorph, co-crystal, tautomer, stereoisomer, derivative, or prodrug thereof:
or a combination thereof, and optionally an additional antiviral agent.

110. The method of any one of claims 1-4, 6-90, and 96-109, wherein the virus is of the Flaviviridae family.

111. The method of any one of claims 1-4, 6-90, and 96-109, wherein the virus is of the Flavivirus genus.

112. The method of any one of claims 1-4, 6-90, and 96-109, wherein the virus is Dengue virus 2 (DENV2).

113. The method of any one of claims 1-4, 6-90, and 96-109, wherein the virus is Dengue virus 1 (DENV1), Dengue virus 3 (DENV3), Dengue virus 4 (DENV4), or Kedougou virus (KEDV).

114. The method of any one of claims 1-4, 6-90, and 96-109, wherein the virus is yellow fever virus (YFV), West Nile virus (WNV), Japanese encephalitis virus (JEV), or Zika virus, preferably, Zika virus.

115. The method of any one of claims 1-4, 6-90, and 96-109, wherein the virus is a tick-bome virus.

116. The method of any one of claims 1-4, 6-90, and 96-109, wherein the virus is Greek goat encephalitis virus (GGEV), Kadam virus (KADV), Krasnodar virus (KRDV), Mogiana tick virus (MGTV), Ngoye virus (NGOV), Sokuluk virus (SOKV), Spanish sheep encephalomyelitis virus (SSEV), Turkish sheep encephalitis virus (TSE), Absettarov virus, Deer tick virus (DT), Gadgets Gully virus (GGYV), Karshi virus, Kyasanur Forest disease virus (KFDV), Alkhurma hemorrhagic fever virus (ALKV), Langat virus (LGTV), Louping ill virus (LIV), Omsk hemorrhagic fever virus (OHFV), Powassan virus (POWV), Royal Farm virus (RFV), Tick-borne encephalitis virus (TBEV), Kama virus (KAMV), Meaban virus (MEAV), Saumarez Reef virus (SREV), or Tyuleniy virus (TYUV).

117. The method of any one of claims 1-4, 6-90, and 96-109, wherein the virus is a mosquito-bome virus.

118. The method of any one of claims 1-4, 6-90, and 96-109, wherein the virus is Aedes flavivirus, Barkedji virus, Calbertado virus, Cell fusing agent virus, Chaoyang virus, Culex flavivirus, Culex theileri flavivirus, Culiseta flavivirus, Donggang virus, Ilomantsi virus, Kamiti River virus, Lammi virus, Marisma mosquito virus, Nounand virus, Nhumirim virus, Nienokoue virus, Spanish Culex flavivirus, Spanish Ochlerotatus flavivirus, Quang Binh virus, Aroa virus (AROAV), Bussuquara virus (BSQV), Iguape virus (IGUV), Naranjal virus (NJLV), Cacipacore virus (CPCV), Koutango virus (KOUV), Kunjin virus, ilheus virus (ILHV), Japanese encephalitis virus (JEV), Murray Valley encephalitis virus (MVEV), Alfuy virus, Rocio virus (ROCV), St. Louis encephalitis virus (SLEV), Usutu virus (USUV), West Nile virus (WNV), Yaounde virus (YAOV), Kokobera virus (KOKV), New Mapoon virus (NMV), Stratford virus (STRV), Bagaza virus (BAGV), Baiyangdian virus (BYDV), Duck egg drop syndrome virus (DEDSV), Ilheus virus (ILHV), Israel turkey meningoencephalomyelitis virus (ITV), Jiangsu virus (JSV), Layer flavivirus, Ntaya virus (NTAV), Sitiawan virus (STWV), Tembusu virus (TMUV), Spondweni virus (SPOV), Zika virus (ZIKV), Banzi virus (BANV), Bamaga virus (BGV), Bouboui virus (BOUV), Edge Hill virus (EHV), Jugra virus (JUGV), Saboya virus (SABV), Sepik virus (SEPV), Uganda S virus (UGSV), Wesselsbron virus (WESSV), yellow fever virus (YFV), Batu cave virus, Bukulasa bat virus, Nanay virus, Rabensburg virus (RABV), or Sitiawan virus.

119. The method of any one of claims 1-4, 6-90, and 96-109, wherein the virus is Tamana bat virus (TABV), Entebbe bat virus (ENTV), Sokoluk virus, Yokose virus (YOKV), Apoi virus (APOIV), Cowbone Ridge virus (CRV), Jutiapa virus (JUTV), Modoc virus (MODV), Sal Vieja virus (SVV), San Perlita virus (SPV), Bukalasa bat virus (BBV), Carey Island virus (CIV), Dakar bat virus (DBV), Montana myotis leukoencephalitis virus (MMLV), Phnom Penh bat virus (PPBV), or Rio Bravo virus (RBV).

120. The method of any one of claims 1-4, 6-90, and 96-109, wherein the virus is Assam virus, Bamaga virus, Cuacua virus, Hanko virus, Mediterranean Ochlerotatus flavivirus, Menghai flavivirus, Nakiwogo virus (NAKV), Ochlerotatus caspius flavivirus, Palm Creek virus, Parramatta River virus, Soybean cyst nematode virus 5, or Xishuangbanna Aedes flavivirus.

121. The method of any one of claims 1-4, 6-90, and 96-109, wherein the virus is Aedes flavivirus, Aedes cinereus flavivirus, Aedes vexans flavivirus, or Culex theileri flavivirus.

122. The method of any one of claims 1-4, 6-90, and 96-109, wherein the virus is of the Hepacivirus genus, Pegivirus genus, or Pestivirus genus.

123. The method of any one of claims 1-4, 6-90, and 96-109, wherein the virus is Hepacivirus A, Hepacivirus B, Hepacivirus C, Hepacivirus D, Hepacivirus E, Hepacivirus F, Hepacivirus G, Hepacivirus H, Hepacivirus I, Hepacivirus J, Hepacivirus K, Hepacivirus L, Hepacivirus M, Hepacivirus N, Pegivirus A, Pegivirus B, Pegivirus C, Pegivirus D, Pegivirus E, Pegivirus F, Pegivirus G, Pegivirus H, Pegivirus I, Pegivirus J, Pegivirus K, or bovine viral diarrhea virus 1.

124. The method of any one of claims 1-4, 6-90, and 96-109, wherein the virus is vesicular stomatitis virus (VSV), vesicular stomatitis virus (VSV) pseudotyped with rabies glycoprotein, vesicular stomatitis virus (VSV) pseudotyped with Ebola glycoprotein, Venezuelan equine encephalitis virus (VEEV), classical swine fever virus, hog cholera virus, papillomavirus, coronavirus, Epstein-Barr virus (EBV), human immunodeficiency virus (HIV), orthomyxovirus, paramyxovirus, arenavirus, bunyavirus, adenovirus, poxvirus, retrovirus, rhabdovirus, picomavirus, or herpesvirus.

125. The method of any one of claims 1-4, 6-90, and 96-124, wherein the virus is in vitro.

126. The method of any one of claims 1-4, 6-90, and 96-125, wherein the antiviral agent is a compound of any one of the preceding claims, or a pharmaceutically acceptable salt, solvate, hydrate, polymorph, co-crystal, tautomer, stereoisomer, isotopically labeled derivative, or prodrug thereof.

127. The method of any one of claims 1-4, 6-90, and 96-126, wherein the antiviral agent is a compound of any one of the preceding claims, or a pharmaceutically acceptable salt thereof.

128. The method of any one of claims 1-4, 6-90, and 96-127, wherein the antiviral agent is a compound of the formula: 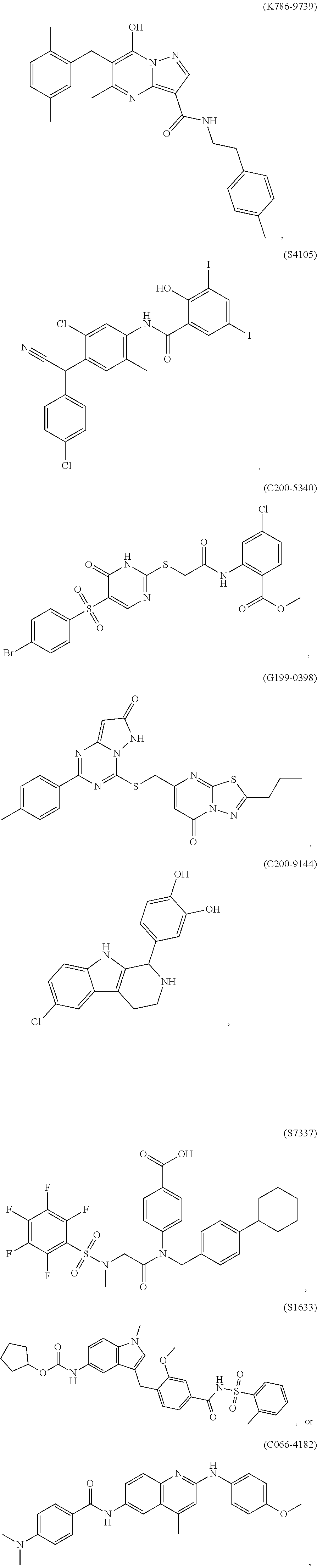
or a pharmaceutically acceptable salt, solvate, hydrate, polymorph, co-crystal, tautomer, stereoisomer, derivative, or prodrug thereof.

129. The method of any one of claims 1-4, 6-90, and 96-127, wherein the antiviral agent is a compound of the formula: 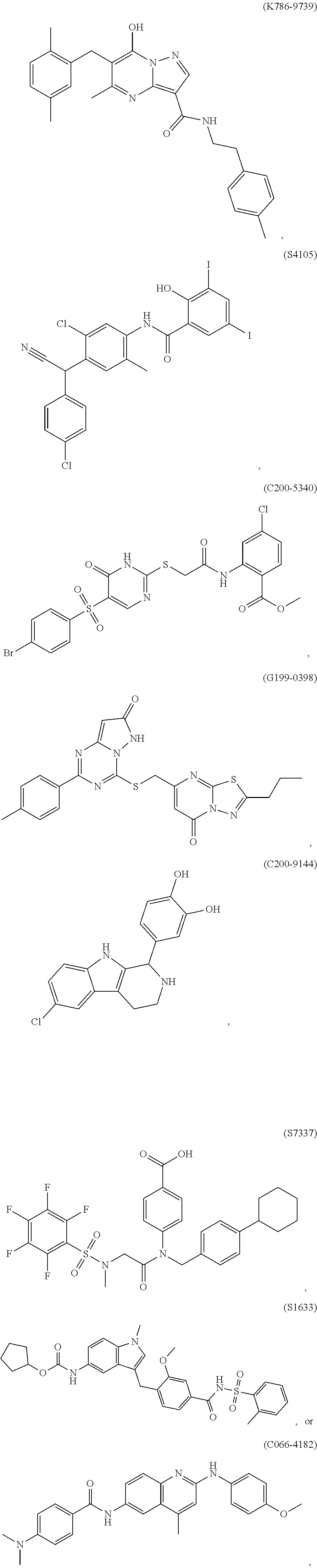
or a pharmaceutically acceptable salt thereof.

130. The method of any one of claims 1-4, 6-90, and 96-127, wherein the antiviral agent is of the formula: 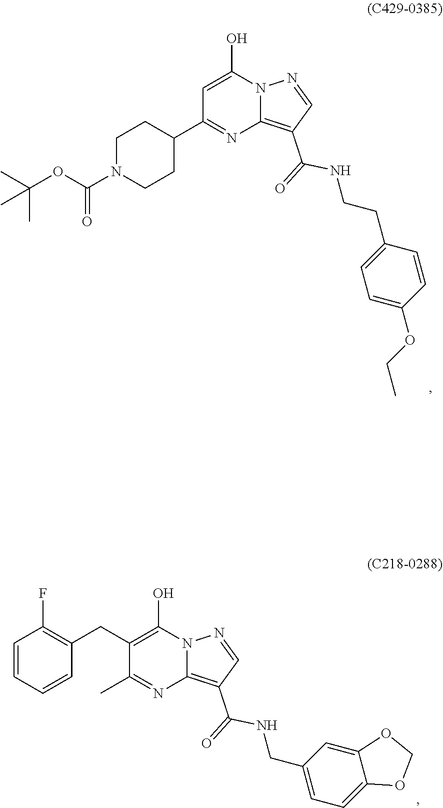 [SMILES: CCOc1ccc(CCNC(=O)c2cnn3c(O)cc(C4CCN(C(=O)OC(C)(C)C)CC4)nc23)cc1 ; Cc1nc2c(C(=O)NCc3ccc4c(c3)OCO4)cnn2c(O)c1Cc1ccccc1F]
or a pharmaceutically acceptable salt, solvate, hydrate, polymorph, co-crystal, tautomer, stereoisomer, derivative, or prodrug thereof.
